SEQUENCE LISTING (219 sequences)

SEQ ID NO: 1 - protein, 10 aa, Artificial Sequence
  Description of Artificial Sequence Synthetic peptide
DPDQLYSTLK

SEQ ID NO: 2 - protein, 20 aa, Artificial Sequence
  Description of Artificial Sequence Synthetic peptide
GLPGGWVTIQDGIYTAHPVR

SEQ ID NO: 3 - protein, 9 aa, Artificial Sequence
  Description of Artificial Sequence Synthetic peptide
SRSDLDYIR

SEQ ID NO: 4 - protein, 18 aa, Artificial Sequence
  Description of Artificial Sequence Synthetic peptide
YKAPTDYCFVLKHPQIQK

SEQ ID NO: 5 - protein, 10 aa, Artificial Sequence
  Description of Artificial Sequence Synthetic peptide
APTDYCFVLK

SEQ ID NO: 6 - protein, 15 aa, Artificial Sequence
  Description of Artificial Sequence Synthetic peptide
TDKKSTIYVGKLDII

SEQ ID NO: 7 - protein, 15 aa, Artificial Sequence
  Description of Artificial Sequence Synthetic peptide
YEGKMETLGENEYFR

SEQ ID NO: 8 - protein, 15 aa, Artificial Sequence
  Description of Artificial Sequence Synthetic peptide
YEGKMETLGENEYFR

SEQ ID NO: 9 - protein, 15 aa, Artificial Sequence
  Description of Artificial Sequence Synthetic peptide
SPSAEDSYVPMSPKG

SEQ ID NO: 10 - protein, 15 aa, Artificial Sequence
  Description of Artificial Sequence Synthetic peptide
TKKFSLDYLALDFNS

SEQ ID NO: 11 - protein, 12 aa, Artificial Sequence
  Description of Artificial Sequence Synthetic peptide
ALYNLGNVYHAK

SEQ ID NO: 12 - protein, 17 aa, Artificial Sequence
  Description of Artificial Sequence Synthetic peptide
LETADAPARLEYYENAR

SEQ ID NO: 13 - protein, 17 aa, Artificial Sequence
  Description of Artificial Sequence Synthetic peptide
LETADAPARLEYYENAR

SEQ ID NO: 14 - protein, 15 aa, Artificial Sequence
  Description of Artificial Sequence Synthetic peptide
KKTANNPYVLMVLYK

SEQ ID NO: 15 - protein, 15 aa, Artificial Sequence
  Description of Artificial Sequence Synthetic peptide
PYVLMVLYKDKVYNI

SEQ ID NO: 16 - protein, 15 aa, Artificial Sequence
  Description of Artificial Sequence Synthetic peptide
IVKKEGPYWNCNSCS

SEQ ID NO: 17 - protein, 14 aa, Artificial Sequence
  Description of Artificial Sequence Synthetic peptide
FLGLLYELEENTDK

SEQ ID NO: 18 - protein, 12 aa, Artificial Sequence
  Description of Artificial Sequence Synthetic peptide
YKAPTDYCLVLK

SEQ ID NO: 19 - protein, 26 aa, Artificial Sequence
  Description of Artificial Sequence Synthetic peptide
LQDLLLHYTAHPLSPYGETLTEPLAR

SEQ ID NO: 20 - protein, 18 aa, Artificial Sequence
  Description of Artificial Sequence Synthetic peptide
TVDFTQDSNYLLTGGQDK

SEQ ID NO: 21 - protein, 14 aa, Artificial Sequence
  Description of Artificial Sequence Synthetic peptide
SNHYDPEEDEEYYR

SEQ ID NO: 22 - protein, 26 aa, Artificial Sequence
  Description of Artificial Sequence Synthetic peptide
LGLALNYSVFYYEIQNAPEQACHLAK

SEQ ID NO: 23 - protein, 16 aa, Artificial Sequence
  Description of Artificial Sequence Synthetic peptide
ARLQKYSTKPGSYIFR

SEQ ID NO: 24 - protein, 32 aa, Artificial Sequence
  Description of Artificial Sequence Synthetic peptide
DATMLKIIVGDVDEPPLFSMPSYLMEVYENAK

SEQ ID NO: 25 - protein, 15 aa, Artificial Sequence
  Description of Artificial Sequence Synthetic peptide
FTAVEDQYYCVDCYK

SEQ ID NO: 26 - protein, 15 aa, Artificial Sequence
  Description of Artificial Sequence Synthetic peptide
FPTNLPKYVKELHLQ

SEQ ID NO: 27 - protein, 15 aa, Artificial Sequence
  Description of Artificial Sequence Synthetic peptide
LLKVQRIYLYHNSLD

SEQ ID NO: 28 - protein, 17 aa, Artificial Sequence
  Description of Artificial Sequence Synthetic peptide
GYIHYVFDLGNGPNVIK

SEQ ID NO: 29 - protein, 8 aa, Artificial Sequence
  Description of Artificial Sequence Synthetic peptide
LGIYEAMK

SEQ ID NO: 30 - protein, 15 aa, Artificial Sequence
  Description of Artificial Sequence Synthetic peptide
SAIYQLEEEYENLLK

SEQ ID NO: 31 - protein, 18 aa, Artificial Sequence
  Description of Artificial Sequence Synthetic peptide
AKCNKYADELAKMELKWK

SEQ ID NO: 32 - protein, 16 aa, Artificial Sequence
  Description of Artificial Sequence Synthetic peptide
PKVFFIQACQGDNYQK

SEQ ID NO: 33 - protein, 12 aa, Artificial Sequence
  Description of Artificial Sequence Synthetic peptide
NADGYIDLDELK

SEQ ID NO: 34 - protein, 19 aa, Artificial Sequence
  Description of Artificial Sequence Synthetic peptide
TMCPLLSMKPGEYSYFSPR

SEQ ID NO: 35 - protein, 14 aa, Artificial Sequence
  Description of Artificial Sequence Synthetic peptide
DLAYCVSQLPLTER

SEQ ID NO: 36 - protein, 29 aa, Artificial Sequence
  Description of Artificial Sequence Synthetic peptide
LTYPTCGIGYPVSSAFAFQNTFPHSISAK

SEQ ID NO: 37 - protein, 17 aa, Artificial Sequence
  Description of Artificial Sequence Synthetic peptide
NLEGYVGFANLPNQVYR

SEQ ID NO: 38 - protein, 27 aa, Artificial Sequence
  Description of Artificial Sequence Synthetic peptide
VVETDPSPYCIVAPDTVIHCEGEPIKR

SEQ ID NO: 39 - protein, 15 aa, Artificial Sequence
  Description of Artificial Sequence Synthetic peptide
ALEYTIYNQELNETR

SEQ ID NO: 40 - protein, 15 aa, Artificial Sequence
  Description of Artificial Sequence Synthetic peptide
KEDSNTIYCSVQKPK

SEQ ID NO: 41 - protein, 15 aa, Artificial Sequence
  Description of Artificial Sequence Synthetic peptide
LRRTLMRYVNLTSLL

SEQ ID NO: 42 - protein, 15 aa, Artificial Sequence
  Description of Artificial Sequence Synthetic peptide
RSVSTAVYKRFPTMD

SEQ ID NO: 43 - protein, 7 aa, Artificial Sequence
  Description of Artificial Sequence Synthetic peptide
NVNAPYR

SEQ ID NO: 44 - protein, 11 aa, Artificial Sequence
  Description of Artificial Sequence Synthetic peptide
WTEYGLTFTEK

SEQ ID NO: 45 - protein, 29 aa, Artificial Sequence
  Description of Artificial Sequence Synthetic peptide
MKEIAEAYLGGKVHSAVITVPAYFNDSQR

SEQ ID NO: 46 - protein, 10 aa, Artificial Sequence
  Description of Artificial Sequence Synthetic peptide
SIYYITGESK

SEQ ID NO: 47 - protein, 12 aa, Artificial Sequence
  Description of Artificial Sequence Synthetic peptide
KDFDTALKHYDK

SEQ ID NO: 48 - protein, 10 aa, Artificial Sequence
  Description of Artificial Sequence Synthetic peptide
NIYYLCAPNR

SEQ ID NO: 49 - protein, 30 aa, Artificial Sequence
  Description of Artificial Sequence Synthetic peptide
QKEFPPAMKLLYSVEHEKPLYLSFGRPENK

SEQ ID NO: 50 - protein, 13 aa, Artificial Sequence
  Description of Artificial Sequence Synthetic peptide
VTHYKQYPPNTSK

SEQ ID NO: 51 - protein, 16 aa, Artificial Sequence
  Description of Artificial Sequence Synthetic peptide
QYPPNTSKVYSYFECR

SEQ ID NO: 52 - protein, 15 aa, Artificial Sequence
  Description of Artificial Sequence Synthetic peptide
RAILDHTYSKHVKSE

SEQ ID NO: 53 - protein, 22 aa, Artificial Sequence
  Description of Artificial Sequence Synthetic peptide
AVTGYTDPYTGQQISLFQAMQK

SEQ ID NO: 54 - protein, 16 aa, Artificial Sequence
  Description of Artificial Sequence Synthetic peptide
VNNVTSERLYRELYHK

SEQ ID NO: 55 - protein, 24 aa, Artificial Sequence
  Description of Artificial Sequence Synthetic peptide
VTKFKAPQYTMAARVEPPGDKTLK

SEQ ID NO: 56 - protein, 15 aa, Artificial Sequence
  Description of Artificial Sequence Synthetic peptide
KRHMDGMYGPPAKRH

SEQ ID NO: 57 - protein, 27 aa, Artificial Sequence
  Description of Artificial Sequence Synthetic peptide
GFGSYHPSAYSPFHFQPSAASLTANLR

SEQ ID NO: 58 - protein, 13 aa, Artificial Sequence
  Description of Artificial Sequence Synthetic peptide
TVYQFCSPSCWTK

SEQ ID NO: 59 - protein, 12 aa, Artificial Sequence
  Description of Artificial Sequence Synthetic peptide
GAVYSMVEFNGK

SEQ ID NO: 60 - protein, 15 aa, Artificial Sequence
  Description of Artificial Sequence Synthetic peptide
CIRKFLSYKTQCPTC

SEQ ID NO: 61 - protein, 17 aa, Artificial Sequence
  Description of Artificial Sequence Synthetic peptide
YAEYSFTSLPVPESNLR

SEQ ID NO: 62 - protein, 8 aa, Artificial Sequence
  Description of Artificial Sequence Synthetic peptide
DLKYYRPT

SEQ ID NO: 63 - protein, 10 aa, Artificial Sequence
  Description of Artificial Sequence Synthetic peptide
DLKYYRPTEV

SEQ ID NO: 64 - protein, 19 aa, Artificial Sequence
  Description of Artificial Sequence Synthetic peptide
CHDYYTTEFLYNLYSSEGK

SEQ ID NO: 65 - protein, 9 aa, Artificial Sequence
  Description of Artificial Sequence Synthetic peptide
HLYRGIFPV

SEQ ID NO: 66 - protein, 9 aa, Artificial Sequence
  Description of Artificial Sequence Synthetic peptide
GGPLDGTYR

SEQ ID NO: 67 - protein, 23 aa, Artificial Sequence
  Description of Artificial Sequence Synthetic peptide
NSVTGGTAAFEPSVDYCVVKIPR

SEQ ID NO: 68 - protein, 12 aa, Artificial Sequence
  Description of Artificial Sequence Synthetic peptide
EGWPSSAYGVTK

SEQ ID NO: 69 - protein, 30 aa, Artificial Sequence
  Description of Artificial Sequence Synthetic peptide
SPEEGAETPVYLALLPPDAEGPHGQFVSEK

SEQ ID NO: 70 - protein, 14 aa, Artificial Sequence
  Description of Artificial Sequence Synthetic peptide
TKEYTACELMNIYK

SEQ ID NO: 71 - protein, 24 aa, Artificial Sequence
  Description of Artificial Sequence Synthetic peptide
SYSYVCGISSKDEPDWESLIFLAR

SEQ ID NO: 72 - protein, 16 aa, Artificial Sequence
  Description of Artificial Sequence Synthetic peptide
KLYESLLVGSLPDMNK

SEQ ID NO: 73 - protein, 23 aa, Artificial Sequence
  Description of Artificial Sequence Synthetic peptide
ANCHKLFPYALAFMPPGYEEDMK

SEQ ID NO: 74 - protein, 13 aa, Artificial Sequence
  Description of Artificial Sequence Synthetic peptide
GNTAAYLLYAFTR

SEQ ID NO: 75 - protein, 20 aa, Artificial Sequence
  Description of Artificial Sequence Synthetic peptide
DQELYFFHELSPGSCFFLPK

SEQ ID NO: 76 - protein, 16 aa, Artificial Sequence
  Description of Artificial Sequence Synthetic peptide
SAYDSTMETMNYAQIR

SEQ ID NO: 77 - protein, 33 aa, Artificial Sequence
  Description of Artificial Sequence Synthetic peptide
GDVGQPGPNGIPSDTLHPIIAPTGVTFHPDQYK

SEQ ID NO: 78 - protein, 15 aa, Artificial Sequence
  Description of Artificial Sequence Synthetic peptide
SQRKLMRYHSVSTET

SEQ ID NO: 79 - protein, 15 aa, Artificial Sequence
  Description of Artificial Sequence Synthetic peptide
ELLSNVDEGIYQLEK

SEQ ID NO: 80 - protein, 12 aa, Artificial Sequence
  Description of Artificial Sequence Synthetic peptide
GVTQYYAYVTER

SEQ ID NO: 81 - protein, 10 aa, Artificial Sequence
  Description of Artificial Sequence Synthetic peptide
NFLYAWCGKR

SEQ ID NO: 82 - protein, 12 aa, Artificial Sequence
  Description of Artificial Sequence Synthetic peptide
DFCQGNQLNYYK

SEQ ID NO: 83 - protein, 15 aa, Artificial Sequence
  Description of Artificial Sequence Synthetic peptide
LTSSAEDEVETTYSR

SEQ ID NO: 84 - protein, 21 aa, Artificial Sequence
  Description of Artificial Sequence Synthetic peptide
LQEGYDHSYYFIATFITDHIR

SEQ ID NO: 85 - protein, 21 aa, Artificial Sequence
  Description of Artificial Sequence Synthetic peptide
LQEGYDHSYYFIATFITDHIR

SEQ ID NO: 86 - protein, 15 aa, Artificial Sequence
  Description of Artificial Sequence Synthetic peptide
MLESYKKYILVSLIL

SEQ ID NO: 87 - protein, 29 aa, Artificial Sequence
  Description of Artificial Sequence Synthetic peptide
VTFEYRQLEPYELENPNGNSIGEFCLSGL

SEQ ID NO: 88 - protein, 26 aa, Artificial Sequence
  Description of Artificial Sequence Synthetic peptide
SQLLLVNSTQDNNYGCVPLLEDSETR

SEQ ID NO: 89 - protein, 17 aa, Artificial Sequence
  Description of Artificial Sequence Synthetic peptide
NPRPGEENGKDYYFVTR

SEQ ID NO: 90 - protein, 12 aa, Artificial Sequence
  Description of Artificial Sequence Synthetic peptide
DPVPYSIINNYF

SEQ ID NO: 91 - protein, 29 aa, Artificial Sequence
  Description of Artificial Sequence Synthetic peptide
PLLVFVNPKSGGNQGTKVLQMFMWYLNPR

SEQ ID NO: 92 - protein, 10 aa, Artificial Sequence
  Description of Artificial Sequence Synthetic peptide
FKEYCPMVFR

SEQ ID NO: 93 - protein, 15 aa, Artificial Sequence
  Description of Artificial Sequence Synthetic peptide
DLVTEDGADPNPYVK

SEQ ID NO: 94 - protein, 13 aa, Artificial Sequence
  Description of Artificial Sequence Synthetic peptide
MLEYDPAKRITLR

SEQ ID NO: 95 - protein, 15 aa, Artificial Sequence
  Description of Artificial Sequence Synthetic peptide
TPSSNTLDDYMSCFR

SEQ ID NO: 96 - protein, 15 aa, Artificial Sequence
  Description of Artificial Sequence Synthetic peptide
TACGTPGYVAPEVLA

SEQ ID NO: 97 - protein, 11 aa, Artificial Sequence
  Description of Artificial Sequence Synthetic peptide
YQQGDFGYCPR

SEQ ID NO: 98 - protein, 16 aa, Artificial Sequence
  Description of Artificial Sequence Synthetic peptide
IFYQTCRAVQHMHRQK

SEQ ID NO: 99 - protein, 25 aa, Artificial Sequence
  Description of Artificial Sequence Synthetic peptide
ASTDVAGQAVTINLVPTEEQAKPYR

SEQ ID NO: 100 - protein, 17 aa, Artificial Sequence
  Description of Artificial Sequence Synthetic peptide
KDEQVEKENTYTSYLDK

SEQ ID NO: 101 - protein, 13 aa, Artificial Sequence
  Description of Artificial Sequence Synthetic peptide
LGEGTYATVFKGR

SEQ ID NO: 102 - protein, 15 aa, Artificial Sequence
  Description of Artificial Sequence Synthetic peptide
QLDYEWKVVNPYYLR

SEQ ID NO: 103 - protein, 15 aa, Artificial Sequence
  Description of Artificial Sequence Synthetic peptide
QLDYEWKVVNPYYLR

SEQ ID NO: 104 - protein, 15 aa, Artificial Sequence
  Description of Artificial Sequence Synthetic peptide
QLDYEWKVVNPYYLR

SEQ ID NO: 105 - protein, 11 aa, Artificial Sequence
  Description of Artificial Sequence Synthetic peptide
DNWTDAEGYYR

SEQ ID NO: 106 - protein, 14 aa, Artificial Sequence
  Description of Artificial Sequence Synthetic peptide
MKLKIGDPSLYFQK

SEQ ID NO: 107 - protein, 15 aa, Artificial Sequence
  Description of Artificial Sequence Synthetic peptide
SGAILPQYDSYSKKS

SEQ ID NO: 108 - protein, 15 aa, Artificial Sequence
  Description of Artificial Sequence Synthetic peptide
ILPQYDSYSKKSCGS

SEQ ID NO: 109 - protein, 10 aa, Artificial Sequence
  Description of Artificial Sequence Synthetic peptide
DIYRASYYRK

SEQ ID NO: 110 - protein, 12 aa, Artificial Sequence
  Description of Artificial Sequence Synthetic peptide
FPGIWAHGDYCR

SEQ ID NO: 111 - protein, 15 aa, Artificial Sequence
  Description of Artificial Sequence Synthetic peptide
DIRAKPEYHKFLIGK

SEQ ID NO: 112 - protein, 8 aa, Artificial Sequence
  Description of Artificial Sequence Synthetic peptide
DVAYQYVK

SEQ ID NO: 113 - protein, 25 aa, Artificial Sequence
  Description of Artificial Sequence Synthetic peptide
GAAAGQRDYDLLVVGGGSGGLACAK

SEQ ID NO: 114 - protein, 18 aa, Artificial Sequence
  Description of Artificial Sequence Synthetic peptide
TGYESGEYEMLGEGLGVK

SEQ ID NO: 115 - protein, 9 aa, Artificial Sequence
  Description of Artificial Sequence Synthetic peptide
AEEVEYYYR

SEQ ID NO: 116 - protein, 15 aa, Artificial Sequence
  Description of Artificial Sequence Synthetic peptide
DDLEDELYAQKLKYK

SEQ ID NO: 117 - protein, 34 aa, Artificial Sequence
  Description of Artificial Sequence Synthetic peptide
QEPQPQGPPPAAGAVASYDYLVIGGGSGGLASAR

SEQ ID NO: 118 - protein, 15 aa, Artificial Sequence
  Description of Artificial Sequence Synthetic peptide
VVRCDIRYTGHHAYA

SEQ ID NO: 119 - protein, 15 aa, Artificial Sequence
  Description of Artificial Sequence Synthetic peptide
RYTGHHAYASGCTIS

SEQ ID NO: 120 - protein, 8 aa, Artificial Sequence
  Description of Artificial Sequence Synthetic peptide
SGCTISPY

SEQ ID NO: 121 - protein, 20 aa, Artificial Sequence
  Description of Artificial Sequence Synthetic peptide
KHGVYNPNKIFGVTTLDIVR

SEQ ID NO: 122 - protein, 36 aa, Artificial Sequence
  Description of Artificial Sequence Synthetic peptide
WYGLPAVSNMLLEIGGLEFSACPFSGWYMGTEIGVR

SEQ ID NO: 123 - protein, 21 aa, Artificial Sequence
  Description of Artificial Sequence Synthetic peptide
TIIDKSSENGVDYVIMGMPHR

SEQ ID NO: 124 - protein, 18 aa, Artificial Sequence
  Description of Artificial Sequence Synthetic peptide
IIDINYYPVPEACLSNKR

SEQ ID NO: 125 - protein, 15 aa, Artificial Sequence
  Description of Artificial Sequence Synthetic peptide
AKYPDYEVTWANDGY

SEQ ID NO: 126 - protein, 36 aa, Artificial Sequence
  Description of Artificial Sequence Synthetic peptide
LKPEGLHQLLAGFEDKSAYALCTFALSTGDPSQPVR

SEQ ID NO: 127 - protein, 26 aa, Artificial Sequence
  Description of Artificial Sequence Synthetic peptide
CGNQAAIMELDDTLKYSFLQFDPAPR

SEQ ID NO: 128 - protein, 15 aa, Artificial Sequence
  Description of Artificial Sequence Synthetic peptide
IQNTGDYYDLYGGEK

SEQ ID NO: 129 - protein, 24 aa, Artificial Sequence
  Description of Artificial Sequence Synthetic peptide
YINASFIMSYWKPEVMIAAQGPLK

SEQ ID NO: 130 - protein, 8 aa, Artificial Sequence
  Description of Artificial Sequence Synthetic peptide
EYSAPGVR

SEQ ID NO: 131 - protein, 15 aa, Artificial Sequence
  Description of Artificial Sequence Synthetic peptide
VFRGSRPYESGPLEE

SEQ ID NO: 132 - protein, 13 aa, Artificial Sequence
  Description of Artificial Sequence Synthetic peptide
EVDGEGKPYYEVR

SEQ ID NO: 133 - protein, 34 aa, Artificial Sequence
  Description of Artificial Sequence Synthetic peptide
SQLDLELEIENMGAHLNAYTSREQTVYYAKAFSK

SEQ ID NO: 134 - protein, 22 aa, Artificial Sequence
  Description of Artificial Sequence Synthetic peptide
KLAQQYYLVYQEPIPTAQLVQR

SEQ ID NO: 135 - protein, 14 aa, Artificial Sequence
  Description of Artificial Sequence Synthetic peptide
LYQTDPSGTYHAWK

SEQ ID NO: 136 - protein, 24 aa, Artificial Sequence
  Description of Artificial Sequence Synthetic peptide
LYSRRFFPYYVYNIIGGLDEEGKG

SEQ ID NO: 137 - protein, 15 aa, Artificial Sequence
  Description of Artificial Sequence Synthetic peptide
LANMVYQYKGMGLSM

SEQ ID NO: 138 - protein, 17 aa, Artificial Sequence
  Description of Artificial Sequence Synthetic peptide
DKKGPGLYYVDEHGTRL

SEQ ID NO: 139 - protein, 28 aa, Artificial Sequence
  Description of Artificial Sequence Synthetic peptide
AVDQNTAIVGSTTGSNYYVRILSTIDRE

SEQ ID NO: 140 - protein, 31 aa, Artificial Sequence
  Description of Artificial Sequence Synthetic peptide
GWVLGPNNYYSFASQQQKPEDTTIPSTELAK

SEQ ID NO: 141 - protein, 31 aa, Artificial Sequence; identical to SEQ ID NO: 140
  Description of Artificial Sequence Synthetic peptide

SEQ ID NO: 142 - protein, 11 aa, Artificial Sequence
  Description of Artificial Sequence Synthetic peptide
ALEYNSKRTPR

SEQ ID NO: 143 - protein, 11 aa, Artificial Sequence
  Description of Artificial Sequence Synthetic peptide
DSPINANLRYR

SEQ ID NO: 144 - protein, 21 aa, Artificial Sequence
  Description of Artificial Sequence Synthetic peptide
IVSVFYTMVIPMLNPVVYSLR

SEQ ID NO: 145 - protein, 30 aa, Artificial Sequence
  Description of Artificial Sequence Synthetic peptide
DMRTTTNLYLGSMAVSDLLILLGLPFDLYR

SEQ ID NO: 146 - protein, 15 aa, Artificial Sequence
  Description of Artificial Sequence Synthetic peptide
SVKMKMKYYIEYMES

SEQ ID NO: 147 - protein, 15 aa, Artificial Sequence
  Description of Artificial Sequence Synthetic peptide
LYIFDSSYGEHPKRR

SEQ ID NO: 148 - protein, 22 aa, Artificial Sequence
  Description of Artificial Sequence Synthetic peptide
YERPYKPVVLLNAQEGWSAQEK

SEQ ID NO: 149 - protein, 15 aa, Artificial Sequence
  Description of Artificial Sequence Synthetic peptide
GSPGNTVYAQVTRPM

SEQ ID NO: 150 - protein, 15 aa, Artificial Sequence
  Description of Artificial Sequence Synthetic peptide
KNDSMTIYSIVNHSR

SEQ ID NO: 151 - protein, 13 aa, Artificial Sequence
  Description of Artificial Sequence Synthetic peptide
EISEGDEVEVYSR

SEQ ID NO: 152 - protein, 11 aa, Artificial Sequence
  Description of Artificial Sequence Synthetic peptide
NPNGPYPYTLK

SEQ ID NO: 153 - protein, 11 aa, Artificial Sequence
  Description of Artificial Sequence Synthetic peptide
NPNGPYPYTLK

SEQ ID NO: 154 - protein, 18 aa, Artificial Sequence
  Description of Artificial Sequence Synthetic peptide
ASAALLDKLYALGLVPTR

SEQ ID NO: 155 - protein, 18 aa, Artificial Sequence
  Description of Artificial Sequence Synthetic peptide
IPPYHYIHVLDQNSNVSR

SEQ ID NO: 156 - protein, 15 aa, Artificial Sequence
  Description of Artificial Sequence Synthetic peptide
YSPTMGLYLGIYMHA

SEQ ID NO: 157 - protein, 18 aa, Artificial Sequence
  Description of Artificial Sequence Synthetic peptide
RSLGYAYVNFQQPADAER

SEQ ID NO: 158 - protein, 14 aa, Artificial Sequence
  Description of Artificial Sequence Synthetic peptide
GFAYIEFSDKESVR

SEQ ID NO: 159 - protein, 20 aa, Artificial Sequence
  Description of Artificial Sequence Synthetic peptide
YYLKNSPDLGPIGGPPNGML

SEQ ID NO: 160 - protein, 19 aa, Artificial Sequence
  Description of Artificial Sequence Synthetic peptide
VAGCSVHKGYAFVQYSNER

SEQ ID NO: 161 - protein, 22 aa, Artificial Sequence
  Description of Artificial Sequence Synthetic peptide
MHPHLSKGYAYVEFENPDEAEK

SEQ ID NO: 162 - protein, 20 aa, Artificial Sequence
  Description of Artificial Sequence Synthetic peptide
MIMDTAGNDPYCFVEFHEHR

SEQ ID NO: 163 - protein, 15 aa, Artificial Sequence
  Description of Artificial Sequence Synthetic peptide
APPKDLMYSVNPTRV

SEQ ID NO: 164 - protein, 22 aa, Artificial Sequence
  Description of Artificial Sequence Synthetic peptide
STANKYQVFFFGTHETAFLGPK

SEQ ID NO: 165 - protein, 16 aa, Artificial Sequence
  Description of Artificial Sequence Synthetic peptide
IPYILKRQLYENKPRR

SEQ ID NO: 166 - protein, 15 aa, Artificial Sequence
  Description of Artificial Sequence Synthetic peptide
RRALDTNYCFSSTEK

SEQ ID NO: 167 - protein, 16 aa, Artificial Sequence
  Description of Artificial Sequence Synthetic peptide
THSGEKPYECYICHAR

SEQ ID NO: 168 - protein, 16 aa, Artificial Sequence
  Description of Artificial Sequence Synthetic peptide
DGTGHYLCNACGLYSK

SEQ ID NO: 169 - protein, 13 aa, Artificial Sequence
  Description of Artificial Sequence Synthetic peptide
AGESLEPAYYDCR

SEQ ID NO: 170 - protein, 13 aa, Artificial Sequence
  Description of Artificial Sequence Synthetic peptide
FINHSCEPNCYSR

SEQ ID NO: 171 - protein, 29 aa, Artificial Sequence
  Description of Artificial Sequence Synthetic peptide
FYEDDENGWQAFGDFSPTDVHKQYAIVFR

SEQ ID NO: 172 - protein, 16 aa, Artificial Sequence
  Description of Artificial Sequence Synthetic peptide
LGDLNYLIYVFPDRPK

SEQ ID NO: 173 - protein, 15 aa, Artificial Sequence
  Description of Artificial Sequence Synthetic peptide
AEIYEAFENIYPILK

SEQ ID NO: 174 - protein, 17 aa, Artificial Sequence
  Description of Artificial Sequence Synthetic peptide
THTGERPYYCTEPGCGR

SEQ ID NO: 175 - protein, 15 aa, Artificial Sequence
  Description of Artificial Sequence Synthetic peptide
IHSGETPYACPVCGK

SEQ ID NO: 176 - protein, 15 aa, Artificial Sequence
  Description of Artificial Sequence Synthetic peptide
IHTGEKPYKCNLCGK

SEQ ID NO: 177 - protein, 12 aa, Artificial Sequence
  Description of Artificial Sequence Synthetic peptide
EYNEYENIKLER

SEQ ID NO: 178 - protein, 12 aa, Artificial Sequence
  Description of Artificial Sequence Synthetic peptide
EIDDEANSYFQR

SEQ ID NO: 179 - protein, 8 aa, Artificial Sequence
  Description of Artificial Sequence Synthetic peptide
IYQVLDFR

SEQ ID NO: 180 - protein, 14 aa, Artificial Sequence
  Description of Artificial Sequence Synthetic peptide
YSPTSPTYSPTTPK

SEQ ID NO: 181 - protein, 24 aa, Artificial Sequence
  Description of Artificial Sequence Synthetic peptide
FACNGTVIEHPEYGEVIQLQGDQR

SEQ ID NO: 182 - protein, 14 aa, Artificial Sequence
  Description of Artificial Sequence Synthetic peptide
NISMSVEGDYTYLR

SEQ ID NO: 183 - protein, 23 aa, Artificial Sequence
  Description of Artificial Sequence Synthetic peptide
NSTGSGHSAQELPTIRTPADIYR

SEQ ID NO: 184 - protein, 19 aa, Artificial Sequence
  Description of Artificial Sequence Synthetic peptide
TNTAYQCFSILPQSSTHIR

SEQ ID NO: 185 - protein, 12 aa, Artificial Sequence
  Description of Artificial Sequence Synthetic peptide
VTVSPYTEIHLK

SEQ ID NO: 186 - protein, 13 aa, Artificial Sequence
  Description of Artificial Sequence Synthetic peptide
IPWFQYPIIYDIR

SEQ ID NO: 187 - protein, 21 aa, Artificial Sequence
  Description of Artificial Sequence Synthetic peptide
VHLCAAQLLQLTNLEHDVYER

SEQ ID NO: 188 - protein, 22 aa, Artificial Sequence
  Description of Artificial Sequence Synthetic peptide
KPQTVAASSIYSMYTQQQAPGK

SEQ ID NO: 189 - protein, 25 aa, Artificial Sequence
  Description of Artificial Sequence Synthetic peptide
ASFLQSQFPDDAEVYRLIEVTGLAR

SEQ ID NO: 190 - protein, 11 aa, Artificial Sequence
  Description of Artificial Sequence Synthetic peptide
VVACNLYPFVK

SEQ ID NO: 191 - protein, 17 aa, Artificial Sequence
  Description of Artificial Sequence Synthetic peptide
AFTHTAQYDEAISDYFR

SEQ ID NO: 192 - protein, 15 aa, Artificial Sequence
  Description of Artificial Sequence Synthetic peptide
SDYFRKQYSKGISQM

SEQ ID NO: 193 - protein, 18 aa, Artificial Sequence
  Description of Artificial Sequence Synthetic peptide
YGMNPHQTPAQLYTLQPK

SEQ ID NO: 194 - protein, 18 aa, Artificial Sequence
  Description of Artificial Sequence Synthetic peptide
YGMNPHQTPAQLYTLQPK

SEQ ID NO: 195 - protein, 11 aa, Artificial Sequence
  Description of Artificial Sequence Synthetic peptide
EKKYDYDNLPR

SEQ ID NO: 196 - protein, 11 aa, Artificial Sequence
  Description of Artificial Sequence Synthetic peptide
PYNNDIDYYAK

SEQ ID NO: 197 - protein, 11 aa, Artificial Sequence
  Description of Artificial Sequence Synthetic peptide
PYNNDIDYYAK

SEQ ID NO: 198 - protein, 13 aa, Artificial Sequence
  Description of Artificial Sequence Synthetic peptide
FGYTVSEENYMCR

SEQ ID NO: 199 - protein, 8 aa, Artificial Sequence
  Description of Artificial Sequence Synthetic peptide
FYYITLLR

SEQ ID NO: 200 - protein, 15 aa, Artificial Sequence
  Description of Artificial Sequence Synthetic peptide
GIPTDMGYAVAPHHS

SEQ ID NO: 201 - protein, 15 aa, Artificial Sequence
  Description of Artificial Sequence Synthetic peptide
APHHSGVYPVHVQLY

SEQ ID NO: 202 - protein, 18 aa, Artificial Sequence
  Description of Artificial Sequence Synthetic peptide
GASRVRVRPAPRYGSSLR

SEQ ID NO: 203 - protein, 14 aa, Artificial Sequence
  Description of Artificial Sequence Synthetic peptide
WTCCALLAGSEYLK

SEQ ID NO: 204 - protein, 28 aa, Artificial Sequence
  Description of Artificial Sequence Synthetic peptide
IYKKGDIVDIKGMGTVQKGMPHKCYHGK

SEQ ID NO: 205 - protein, 28 aa, Artificial Sequence
  Description of Artificial Sequence Synthetic peptide
IYKKGDIVDIKGMGTVQKGMPHKCYHGK

SEQ ID NO: 206 - protein, 15 aa, Artificial Sequence
  Description of Artificial Sequence Synthetic peptide
LEYMMKCYPEIKEKE

SEQ ID NO: 207 - protein, 9 aa, Artificial Sequence
  Description of Artificial Sequence Synthetic peptide
AWNAYPYCR

SEQ ID NO: 208 - protein, 32 aa, Artificial Sequence
  Description of Artificial Sequence Synthetic peptide
GFLISIPYSASIGGTATLTGTAPNLILLGQLK

SEQ ID NO: 209 - protein, 26 aa, Artificial Sequence
  Description of Artificial Sequence Synthetic peptide
TRMQNQRSTGSFVGELMYKNSFDCFK

SEQ ID NO: 210 - protein, 12 aa, Artificial Sequence
  Description of Artificial Sequence Synthetic peptide
TLIYQMAPAVSK

SEQ ID NO: 211 - protein, 12 aa, Artificial Sequence
  Description of Artificial Sequence Synthetic peptide
DQQLVYSAGVYR

SEQ ID NO: 212 - protein, 15 aa, Artificial Sequence
  Description of Artificial Sequence Synthetic peptide
SNYFEVPLPYFEYFK

SEQ ID NO: 213 - protein, 17 aa, Artificial Sequence
  Description of Artificial Sequence Synthetic peptide
FGGPEMYGYPDPSYLKR

SEQ ID NO: 214 - protein, 21 aa, Artificial Sequence
  Description of Artificial Sequence Synthetic peptide
DLDFSEFVIQPQNESNPELYK

SEQ ID NO: 215 - protein, 19 aa, Artificial Sequence
  Description of Artificial Sequence Synthetic peptide
TPQQEETTYYQTALPGNDR

SEQ ID NO: 216 - protein, 15 aa, Artificial Sequence
  Description of Artificial Sequence Synthetic peptide
ISGTFTHRQGHIYME

SEQ ID NO: 217 - protein, 19 aa, Artificial Sequence
  Description of Artificial Sequence Synthetic peptide
FHPLAANVLASSSYDLTVR

SEQ ID NO: 218 - protein, 11 aa, Artificial Sequence
  Description of Artificial Sequence Synthetic peptide
YESAYQAVVWK

SEQ ID NO: 219 - protein, 15 aa, Artificial Sequence
  Description of Artificial Sequence Synthetic peptide
ESPESEGPIYEGLIL

(19) United States
(12) Patent Application Publication
(10) Pub. No.: US 2010/0173322 A1
(43) Pub. Date: Jul. 8, 2010

(54) Reagents for the detection of protein phosphorylation in anaplastic large cell lymphoma signaling pathways

(75) Inventors: Roberto Polakiewicz, Lexington, MA (US); Ailan Guo, Burlington, MA (US); Albrecht Moritz, Salem, MA (US); Kimberly Lee, Seattle, WA (US); Erik Spek, Cambridge, MA (US); Charles Farnsworth, Concord, MA (US); Francesco Boccalatte, Torino (IT)

Correspondence Address: Nancy Chiu Wilker, Ph.D.;Chief Intellectual Property Counsel, CELL SIGNALING TECHNOLOGY, INC., 3 Trask Lane, Danvers, MA, 01923, US

(73) Assignee: CELL SIGNALING TECHNOLOGY, INC.

(21) Appl. No.: 12/074,876

(22) Filed: Mar. 6, 2008

(51) Int. Cl. G01N 33/53; C07K 16/18; G01N 33/566

(52) U.S. Cl. 435/7.1; 530/387.9; 436/501

(57) ABSTRACT

The invention discloses nearly 219 novel phosphorylation sites identified in signal transduction proteins and pathways underlying Anaplastic Large Cell Lymphoma (ALCL) involving the NPM-ALK translocation/fusion, and provides phosphorylation-site specific antibodies and heavy-isotope labeled peptides (AQUA peptides) for the selective detection and quantification of these phosphorylated sites/proteins, as well as methods of using the reagents for such purpose.

### RELATED APPLICATIONS

This application claims the benefit of, and priority to, PCT serial number PCT/US06/35203, filed Sep. 8, 2006, presently pending, the disclosure of which is incorporated herein, in its entirety, by reference.

### FIELD OF THE INVENTION

The invention relates generally to antibodies and peptide reagents for the detection of protein phosphorylation, and to protein phosphorylation in cancer.

### BACKGROUND OF THE INVENTION

The activation of proteins by post-translational modification represents an important cellular mechanism for regulating most aspects of biological organization and control, including growth, development, homeostasis, and cellular communication. For example, protein phosphorylation plays a critical role in the etiology of many pathological conditions and diseases, including cancer, developmental disorders, autoimmune diseases, and diabetes. In spite of the importance of protein modification, it is not yet well understood at the molecular level. The reasons for this lack of understanding are, first, that the cellular modification system is extraordinarily complex, and second, that the technology necessary to unravel its complexity has not yet been fully developed.

The complexity of protein modification, including phosphorylation, on a proteome-wide scale derives from three factors: the large number of modifying proteins, e.g. kinases, encoded in the genome, the much larger number of sites on substrate proteins that are modified by these enzymes, and the dynamic nature of protein expression during growth, development, disease states, and aging. The human genome encodes, for example, over 520 different protein kinases, making them the most abundant class of enzymes known. See Hunter, Nature 411: 355-65 (2001). Each of these kinases phosphorylates specific serine, threonine, or tyrosine residues located within distinct amino acid sequences, or motifs, contained within different protein substrates. Most kinases phosphorylate many different proteins: it is estimated that one-third of all proteins encoded by the human genome are phosphorylated, and many are phosphorylated at multiple sites by different kinases. See Graves et al., Pharmacol. Ther. 82: 111-21 (1999).

Many of these phosphorylation sites regulate critical biological processes and may prove to be important diagnostic or therapeutic targets for molecular medicine. For example, of the more than 100 dominant oncogenes identified to date, 46 are protein kinases. See Hunter, supra. Oncogenic kinases such as ErbB2 and Jak3, widely expressed in breast tumors and various leukemias, respectively, transform cells to the oncogenic phenotype at least in part because of their ability to phosphorylate cellular proteins. Understanding which proteins are modified by these kinases will greatly expand our understanding of the molecular mechanisms underlying oncogenic transformation. Thus, the ability to identify modification sites, e.g. phosphorylation sites, on a wide variety of cellular proteins is crucially important to understanding the key signaling proteins and pathways implicated in disease progression, for example cancer.

The efficient identification of protein phosphorylation sites relevant to disease has been aided by the recent development of a powerful new class of antibodies, called motif-specific, context-independent antibodies, which are capable of specifically binding short, recurring signaling motifs comprising one or more modified (e.g. phosphorylated) amino acids in many different proteins in which the motif recurs. See U.S. Pat. No. 6,441,140, Comb et al. Many of these powerful new antibodies are now available commercially. See CELL SIGNALING TECHNOLOGY, INC. 2003-04 Catalogue. More recently, a powerful new method for employing such motif-specific antibodies in immunoaffinity techniques coupled with mass spectrometric analysis to rapidly identify modified peptides from complex biological mixtures has been described. See U.S. Patent Publication No. 20030044848, Rush et al.). Such techniques will enable the rapid elucidation of protein activation and phosphorylation events underlying diseases, like cancer, that are driven by disruptions in signal transduction.

One form of cancer, in which underlying signal transduction events are involve but still poorly understood, is Anaplastic Large-Cell Lymphoma (ALCL). ALCL is a sub-type of non-Hodgkin's lymphomas (NHL), which are the 5^{th} most common cancer in the United States, with over 53,000 new diagnoses annually (source: The Leukemia & Lymphoma Society (2004)). Worldwide, more than 166,000 cases of NHL are diagnosed annually, and over 93,000 annual deaths from this group of lymphomas (source: Globocan 2000: Cancer Incidence, Mortality & Prevalence, Version 1.0 (2001)). ALCL, a form of T-cell lymphoma (CD30+), is most prevalent among young children, representing about 15% of all pediatric non-Hodgkin's lymphomas (source: UMDNJ Hematopathology (2004)). It is an aggressive disease that can be either systemic or primary cutaneous, with median survival rates of about 5 years from diagnosis.

Approximately 50% to 60% of all ALCL cases are characterized by a translocation between chromosomes 2p23 and 5q35 leading to an abnormal fusion gene involving the anaplastic lymphoma kinase (ALK) gene and the nucleophosmin gene (NPM), itself involved in nucleo-cytoplasmic trafficking. See, e.g. Ouyang et al., J. Biol. Chem. 278: 300028-300036 (2003); Miller, ProPath “Anaplastic Lymphoma Kinase” (2003). The NPM-ALK fusion protein functions as a constitutively activated protein tyrosine kinase, leading to enhanced cellular proliferation and survival. It has recently been shown that NPM-ALK transgenic mice spontaneously develop T-cell lymphomas including ALCL. See Chiarle et al., Blood 101: 1919-1927 (2003).

A number of downstream signaling protein targets of NPM-ALK have identified as potentially involved in mediating cellular transformation in NPM-ALK positive ALCL, including Shc, IRS-1, Grb2, phospholipase C-γ, PI3-kinase, and Stat3/5. See Ouyang et al. supra; Zamo et al., Oncogene 21: 1038-1047 (2002). NPM-ALK activates the AKT/PI3K anti-apoptotic signaling pathway. See Bai et al., Blood 96: 4319-4327 (2000). Transgenic mice experiments have established that Stat3 and Jak3 are constitutively activated in NPM-ALK positive transgenic mice that develop ALCL. See Chiarle et al., supra. However, despite the identification of some of the downstream targets of NPM-ALK, the molecular mechanisms of contributing to NPM-ALK-mediated oncogenesis in ALCL remain incompletely understood. See Ouyang et al., supra.

A few phosphotyrosine sites that allow NPM-ALK to interact with other signaling proteins have been reported, including Tyr1604, which is a binding site for phospholipase gamma (PLCgamma) (see Bai et al. Mol. Cell. Biol. 18: 6951-6961 (1998), and Tyr1096 and Tyr1507, which are the docking sites for SHC and IRS-1 respectively. See Fujimoto et al., PNAS 93: 4181-4186 (1996). PLCgamma, SHC and IRS-1 are known to be phosphorylated in the context of other signaling cascades (such as the Ras/ERk pathway) and many of their phosphorylation sites have been identified. See Watanabe et al., J. Biol. Chem. 276: 38595-38601 (2001); Law et al., Mol Cell Biol 16: 1305-1315 (1996); van der Geer et al., Curr. Biol. 6: 1432-1444 (1996); White M F, Mol. Cell. Biochem. 182: 3-11 (1998). Another important factor directly phosphorylated by NPM-ALK fusion kinase is STAT3. Phosphorylation of STAT3 at Tyr705 has been shown to be important for oncogenic transformation. See Zamo A. et al. Oncogene 21: 1038-1047 (2002).

Nonetheless, the small number of ALCL-related phosphorylation sites that have been identified to date do not facilitate a complete and accurate understanding of how protein activation within NPM-ALK signaling pathways is driving this disease.

Accordingly, there is a continuing need to unravel the molecular mechanisms of NPM-ALK driven oncogenesis in ALCL, by identifying the downstream signaling proteins mediating cellular transformation in this disease. Identifying particular phosphorylation sites on such signaling proteins and providing new reagents, such as phospho-specific antibodies and AQUA peptides, to detect and quantify them remains particularly important to advancing our understanding of the biology of this disease.

Presently, diagnosis of ALCL is made by tissue biopsy and detection of T-cell markers, such as CD30 and/or CD4. However, mis-diagnosis can occur since some ALCL can be negative for certain markers and/or can be positive for keratin, a marker for carcinoma. Although the NPM-ALK genetic translocation itself can be detected, it is clear that other downstream effectors of ALCL, having diagnostic, predictive, or therapeutic value, remain to be elucidated. Accordingly, identification of downstream signaling molecules and phospho-sites involved in NPM-ALK positive ALCL and development of new reagents to detect and quantify these sites and proteins may lead to improved diagnostic/prognostic markers, as well as novel drug targets, for the detection and treatment of this disease.

### SUMMARY OF THE INVENTION

The invention discloses nearly 219 novel phosphorylation sites identified in signal transduction proteins and pathways underlying Anaplastic Large Cell Lymphoma (ALCL) involving the NPM-ALK translocation/fusion, and provides new reagents, including phosphorylation-site specific antibodies and AQUA peptides, for the selective detection and quantification of these phosphorylated sites/proteins. Also provided are methods of using the reagents of the invention for the detection quantification and profiling of the disclosed phosphorylation sites.

### BRIEF DESCRIPTION OF THE DRAWINGS

This patent or application file contains at least one drawing executed in color. Copies of this patent or patent application publication with color drawing(s) will be provided by the United States Patent Office upon request and payment of the necessary fee.

FIG. 1—Is a diagram broadly depicting the immunoaffinity isolation and mass-spectrometric characterization methodology (IAP) employed to identify the novel phosphorylation sites disclosed herein.

FIG. 2—Is a table (corresponding to Table 1) enumerating the ALCL signaling protein phosphorylation sites disclosed herein: Column A=the abbreviated name of the parent protein; Column B=the full name of the parent protein; Column C=the SwissProt accession number for the protein (human sequence); Column D=the protein type/classification; Column F=the residue (in the parent protein amino acid sequence) at which phosphorylation occurs within the phosphorylation site; Column G=the phosphorylation site sequence encompassing the phosphorylatable residue; (residue at which phosphorylation occurs (and corresponding to the respective entry in Column F) appears in lowercase; and Column I=the ALCL cell line in which the phosphorylation site was discovered.

FIG. 3—is an exemplary mass spectrograph depicting the detection of the tyrosine 1284 phosphorylation site in ALK (see Row 109 in FIG. 2/Table 1), as further described in Example 1 (red and blue indicate ions detected in MS/MS spectrum); Y* (and pY) indicates the phosphorylated tyrosine (shown as lowercase “y” in FIG. 2).

FIG. 4—is an exemplary mass spectrograph depicting the detection of the tyrosine 406 phosphorylation site in ARGHEF2 (see Row 79 in FIG. 2/Table 1), as further described in Example 1 (red and blue indicate ions detected in MS/MS spectrum); Y* (and pY) indicates the phosphorylated tyrosine (shown as lowercase “y” in FIG. 2).

FIG. 5 is an exemplary mass spectrograph depicting the detection of the tyrosine 111 phosphorylation site in IRS4 (see Row 12 in FIG. 2/Table 1), as further described in Example 1 (red and blue indicate ions detected in MS/MS spectrum); Y* (and pY) indicates the phosphorylated tyrosine (shown as lowercase “y” in FIG. 2) and M# (and lowercase “m”) indicates an oxidized methionine also detected.

FIG. 6—is an exemplary mass spectrograph depicting the detection of the tyrosine 466 phosphorylation site in PKM2 (see Row 65 in FIG. 2/Table 1), as further described in Example 1 (red and blue indicate ions detected in MS/MS spectrum); Y* (and pY) indicates the phosphorylated tyrosine (shown as lowercase “y” in FIG. 2).

FIG. 7—is an exemplary mass spectrograph depicting the detection of the tyrosine 284 phosphorylation site in PPP2CB (see Row 127 in FIG. 2/Table 1), as further described in Example 1 (red and blue indicate ions detected in MS/MS spectrum); Y* (and pY) indicates the phosphorylated tyrosine (shown as lowercase “y” in FIG. 2).

FIG. 8—is an exemplary mass spectrograph depicting the detection of the tyrosine 588 phosphorylation site in ACLY (see Row 76 in FIG. 2/Table 1), as further described in Example 1 (red and blue indicate ions detected in MS/MS spectrum); Y* (and pY) indicates the phosphorylated tyrosine (shown as lowercase “y” in FIG. 2).

FIG. 9—is an exemplary mass spectrograph depicting the detection of the tyrosine 3914 phosphorylation site in MLL (see Row 170 in FIG. 2/Table 1), as further described in Example 1 (red and blue indicate ions detected in MS/MS spectrum); Y* (and pY) indicates the phosphorylated tyrosine (shown as lowercase “y” in FIG. 2).

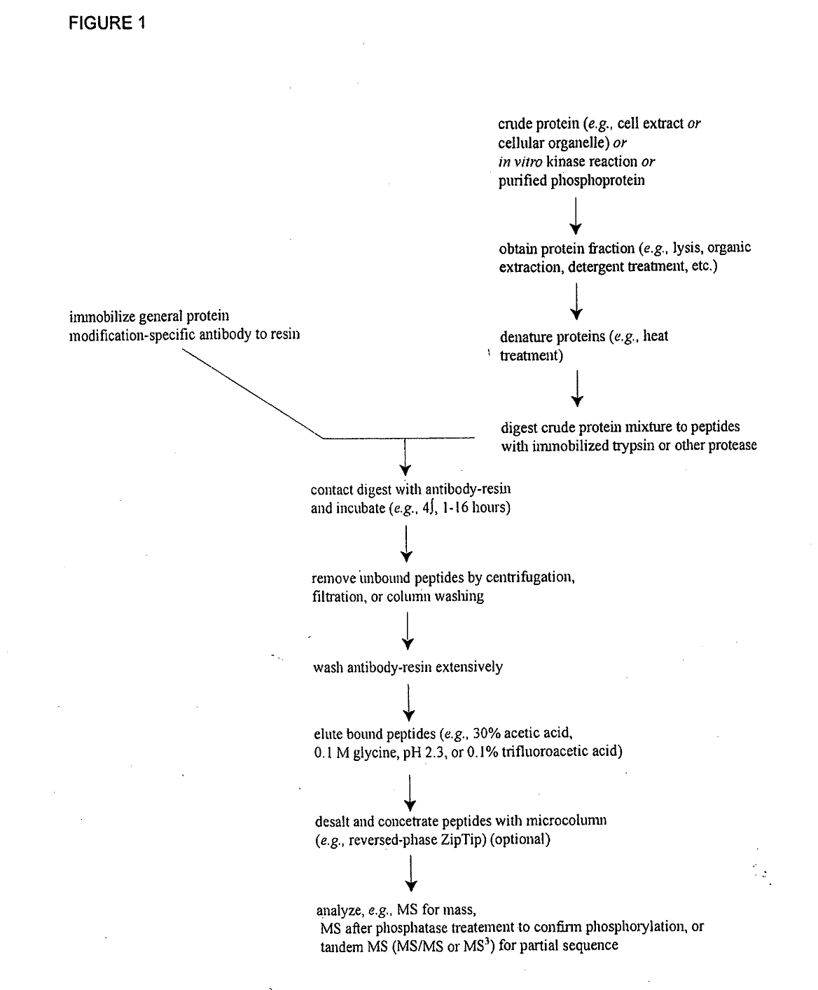

### DETAILED DESCRIPTION OF THE INVENTION

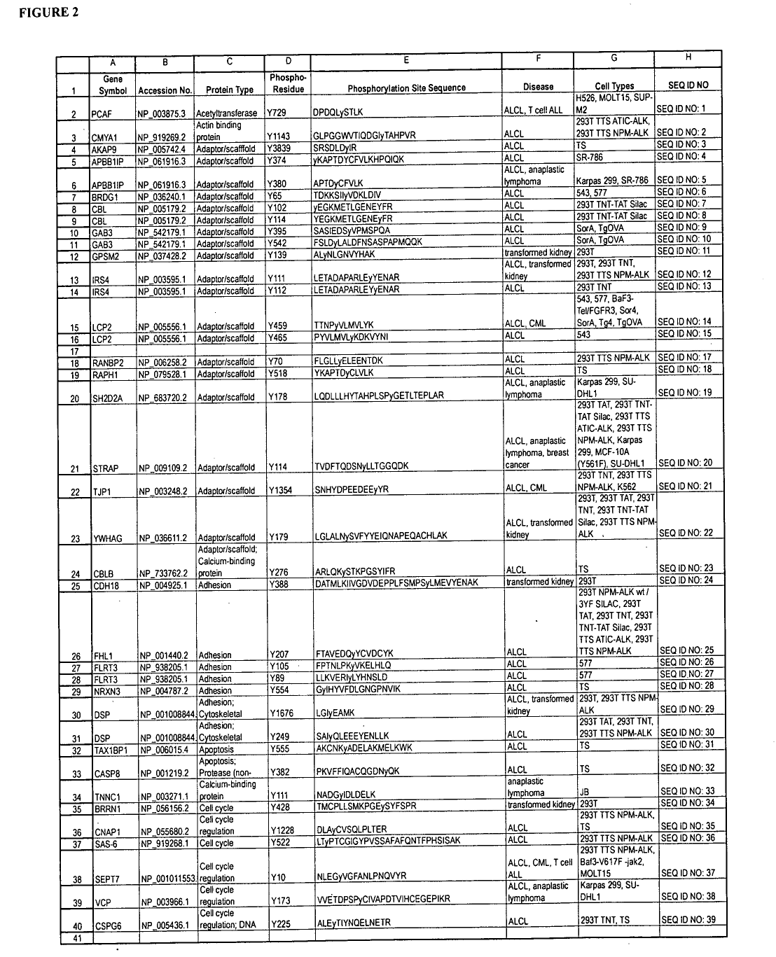

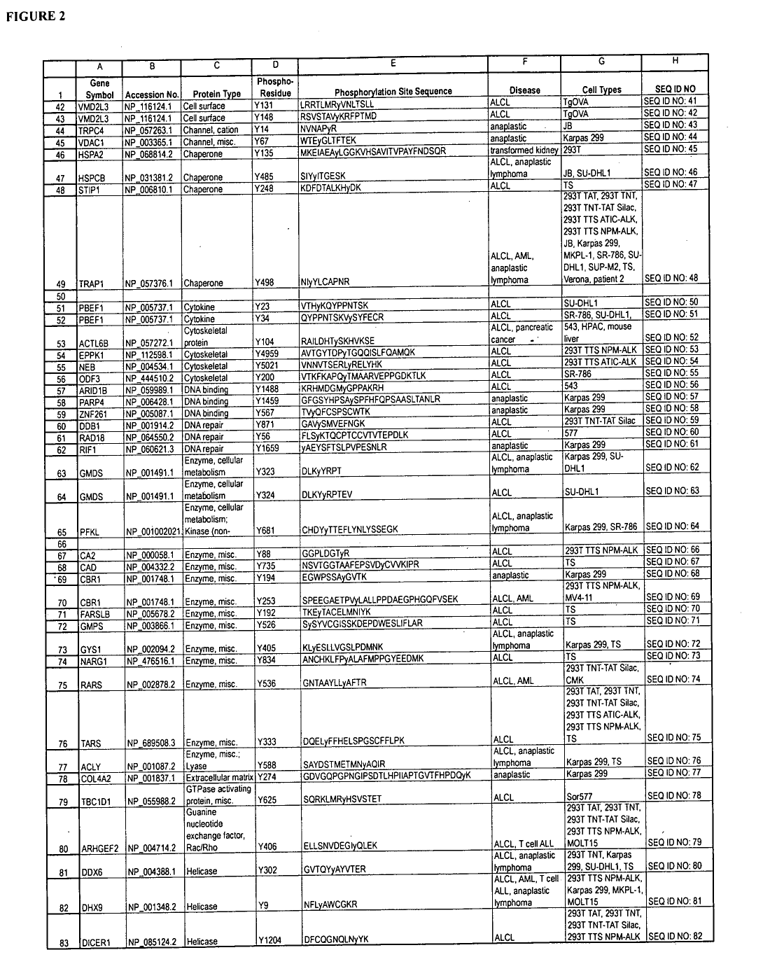

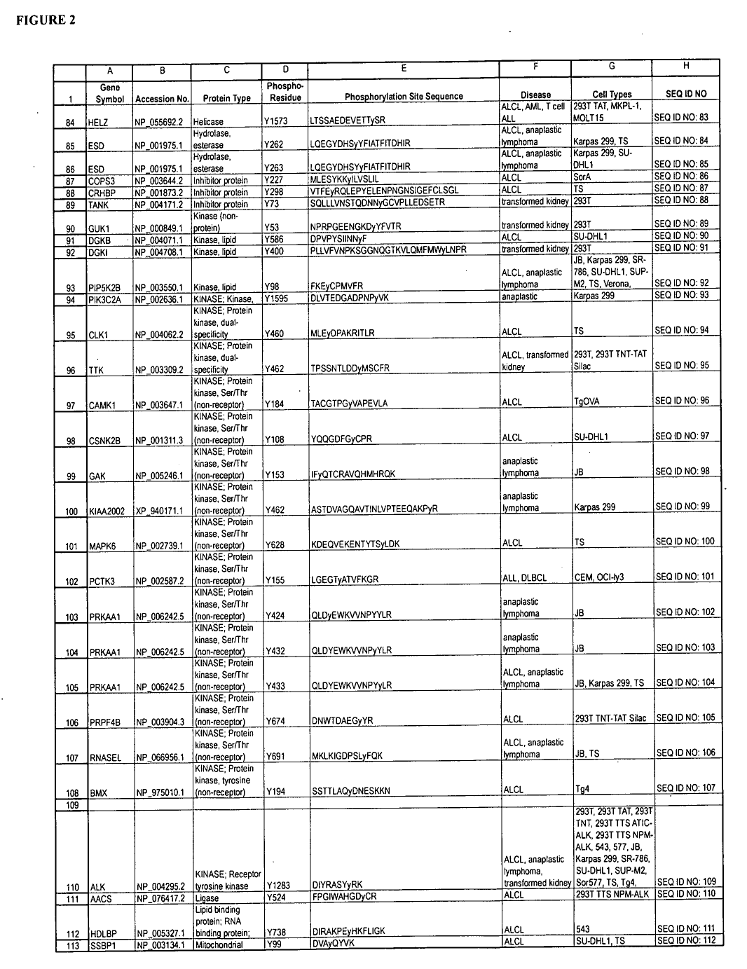

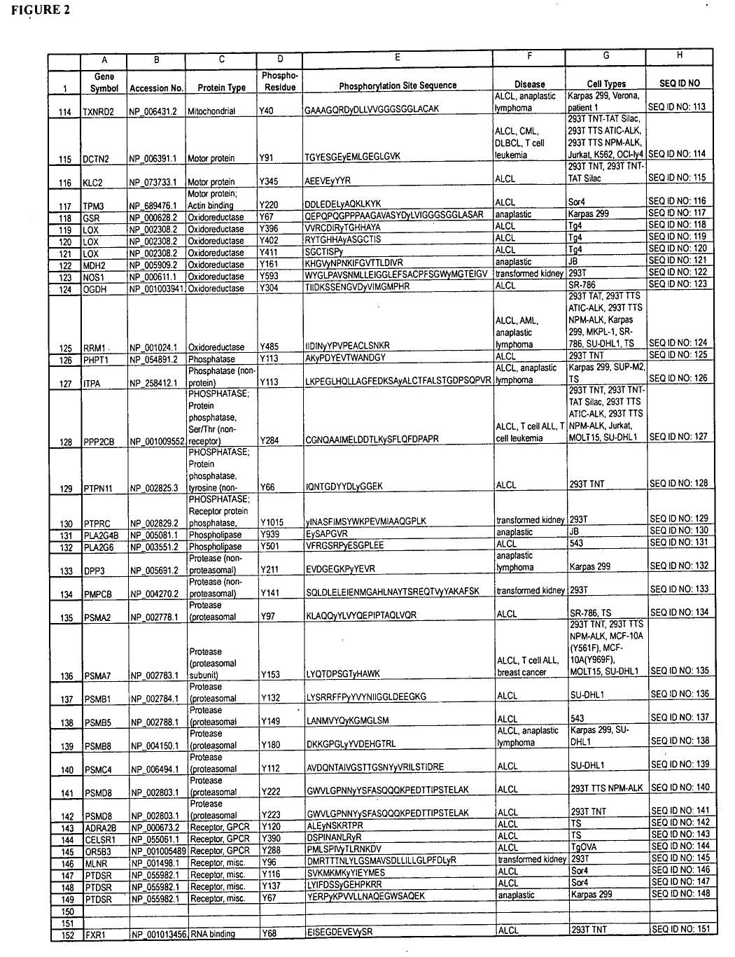

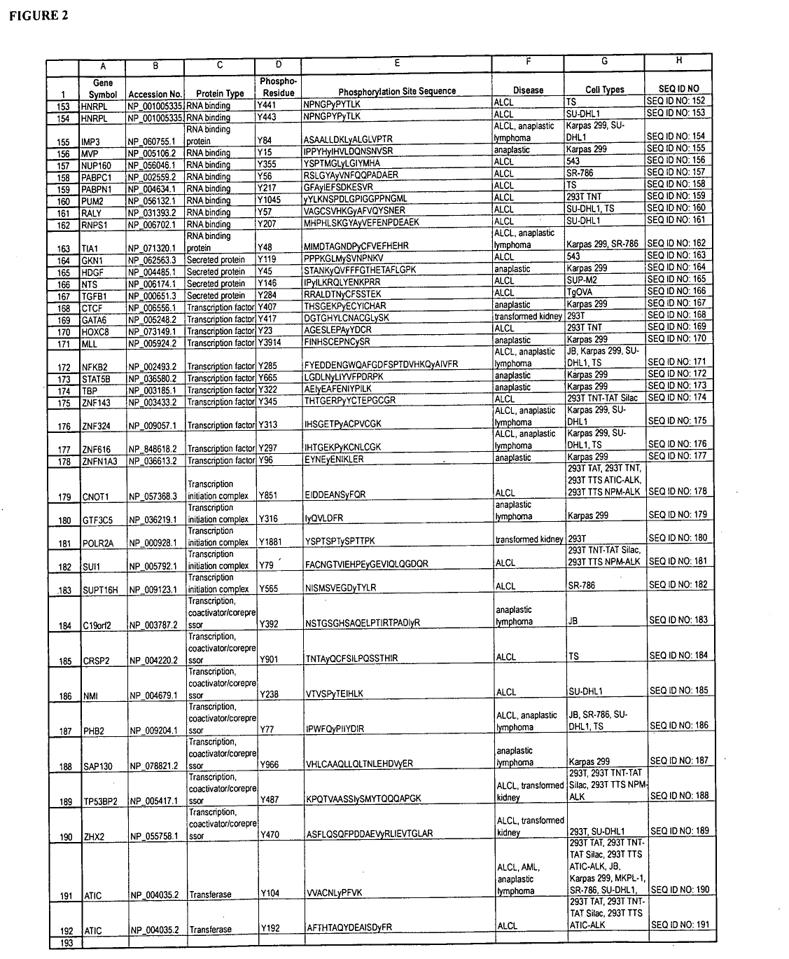

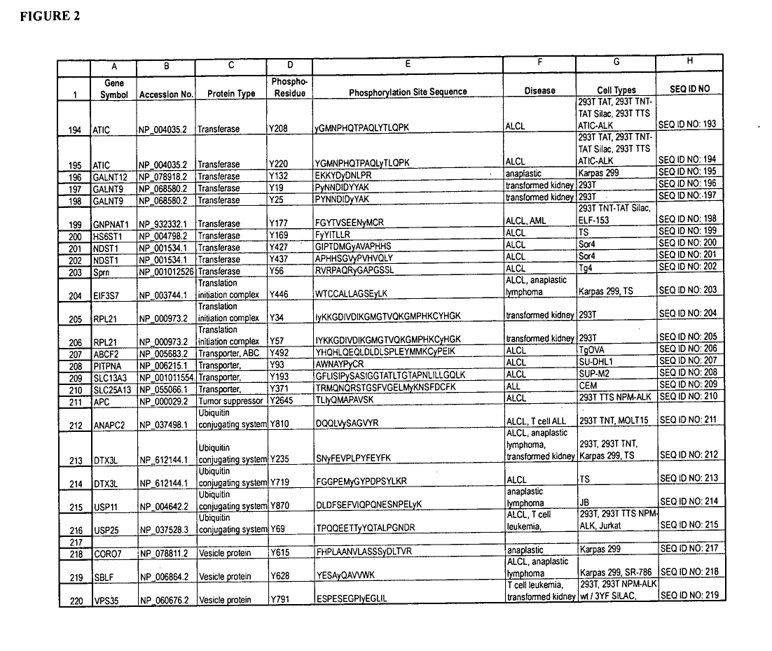

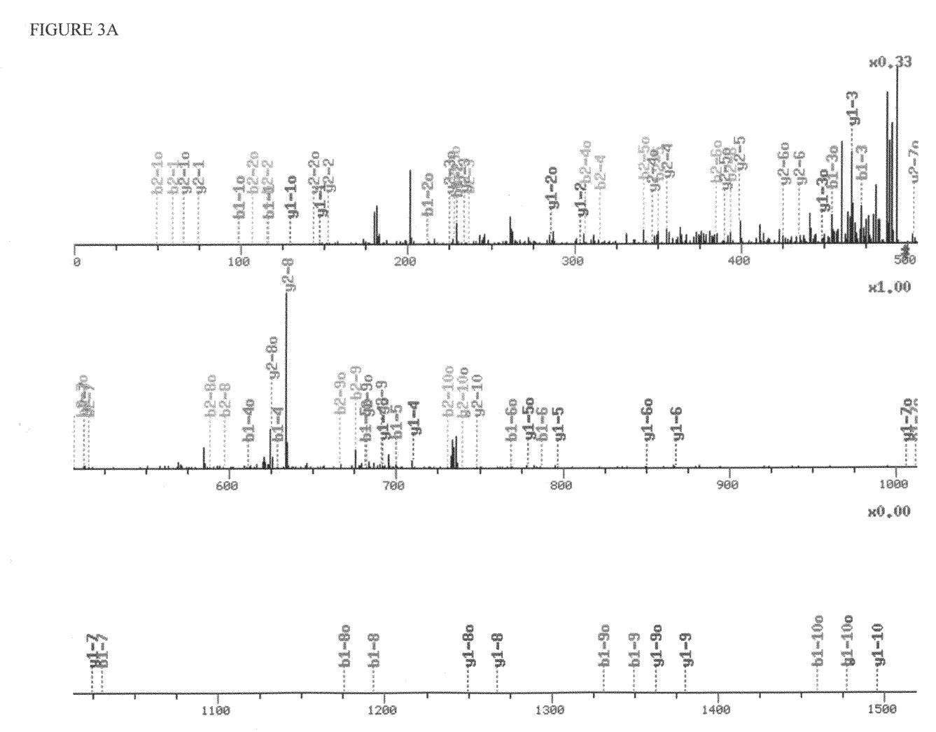

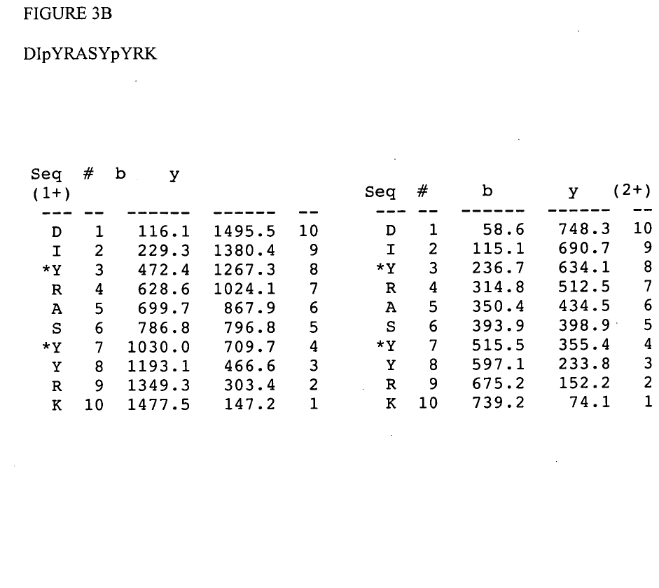

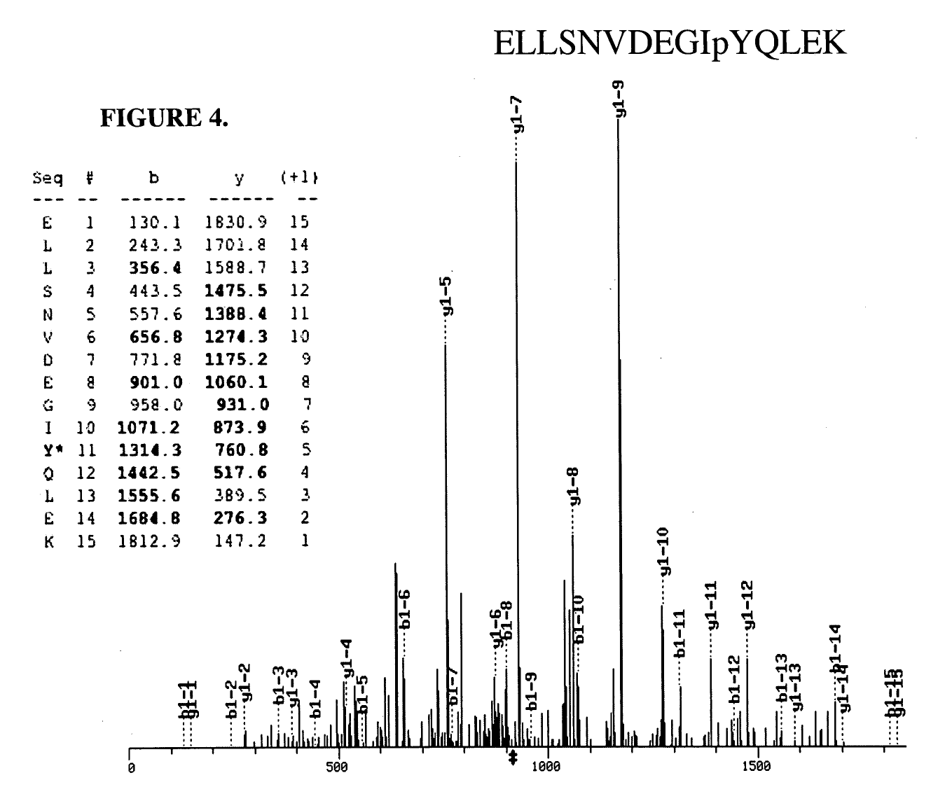

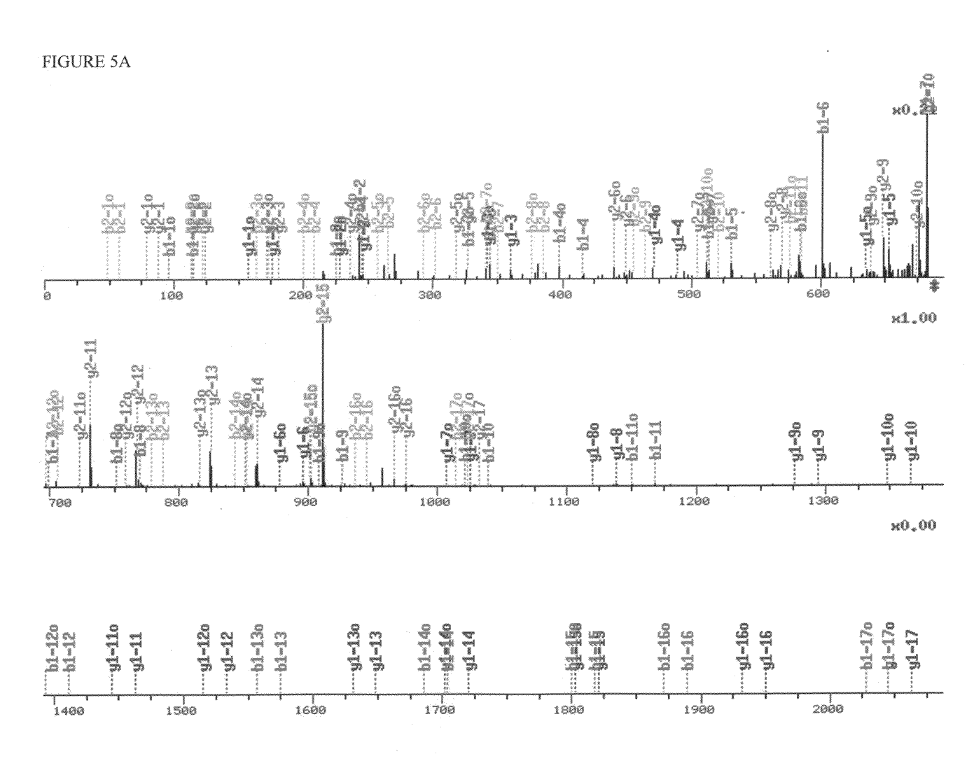

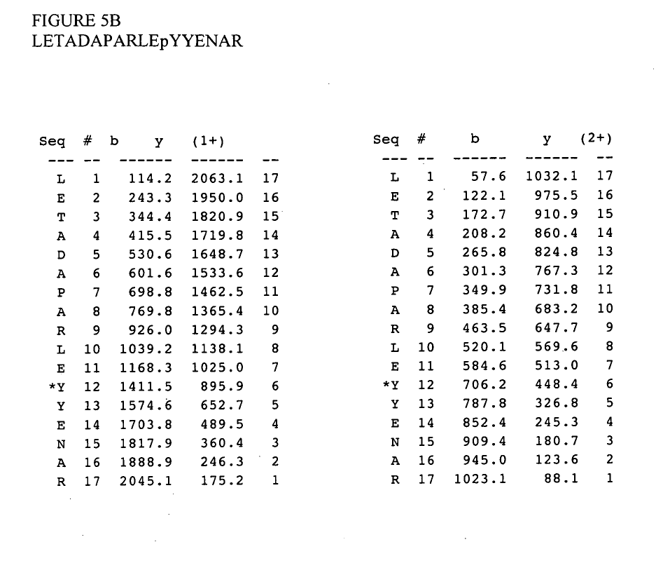

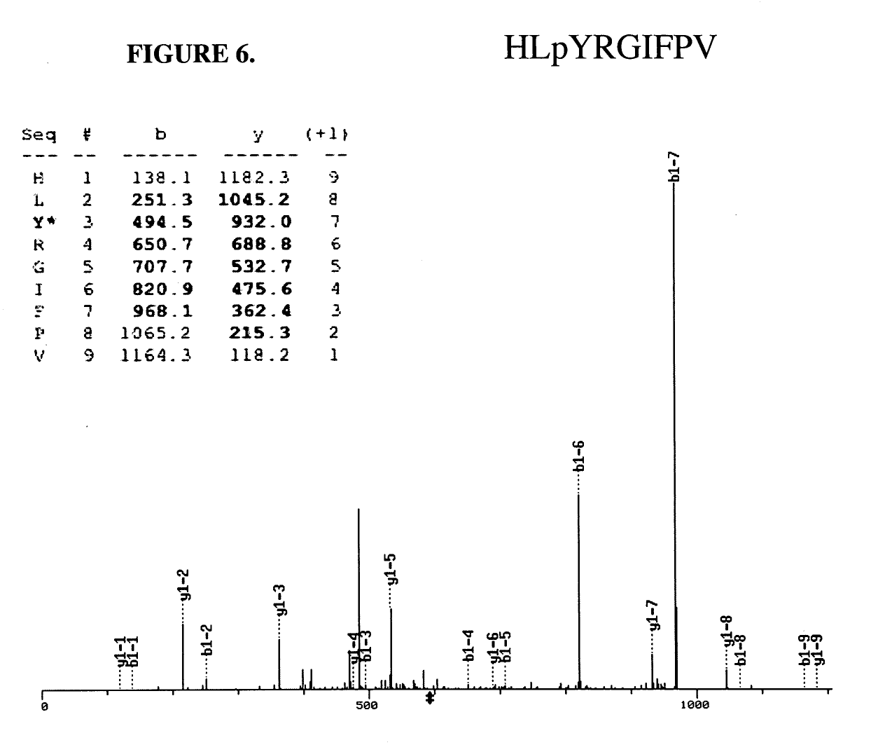

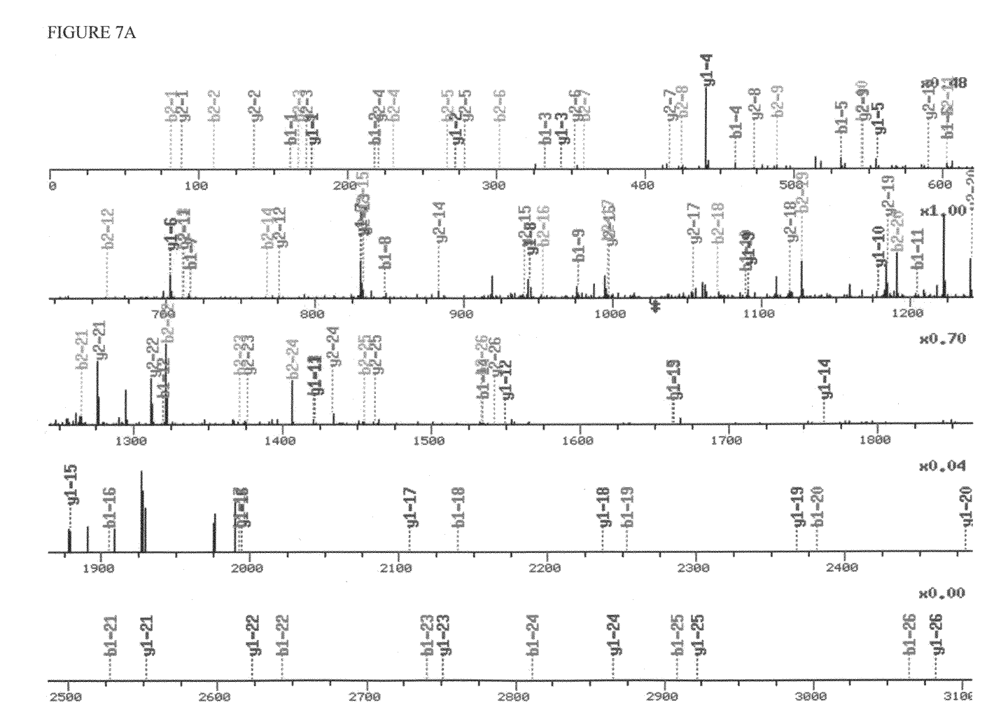

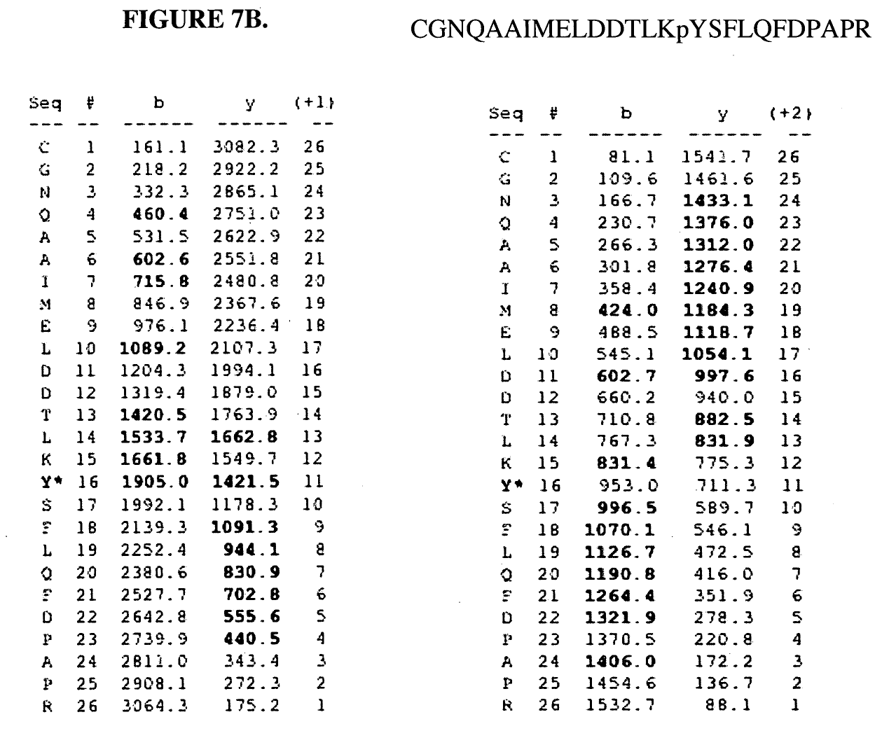

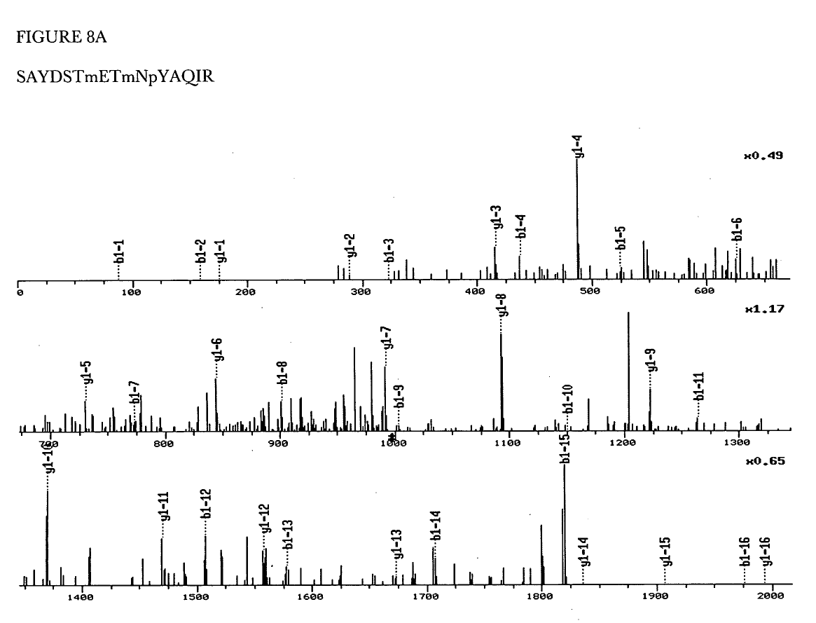

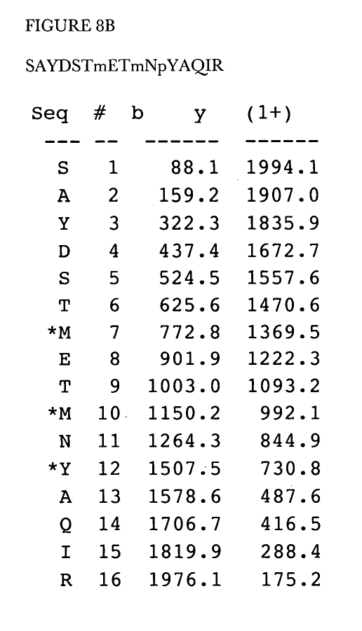

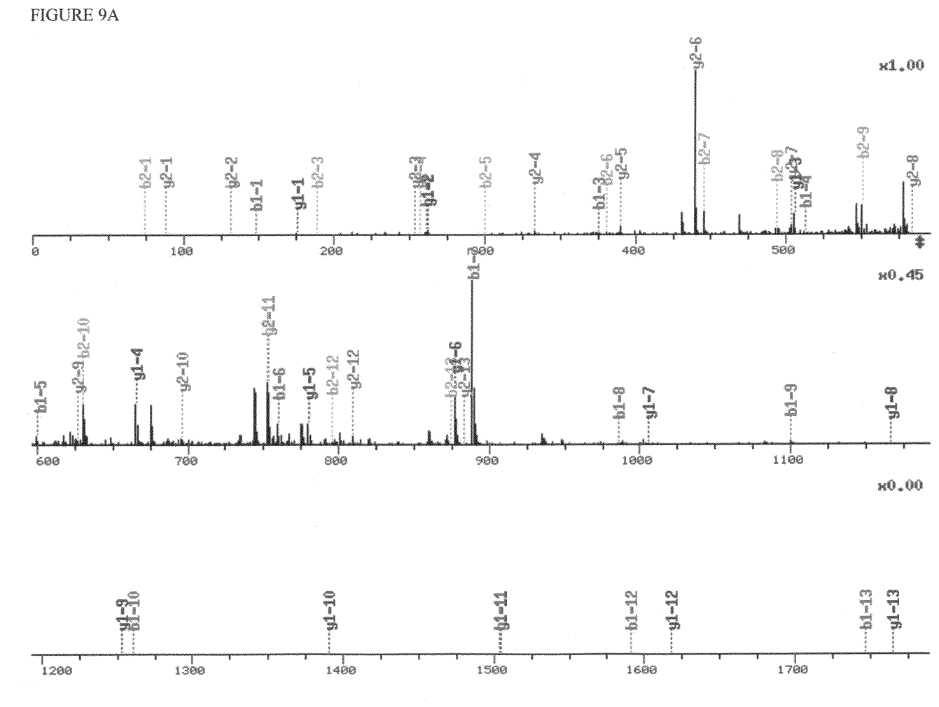

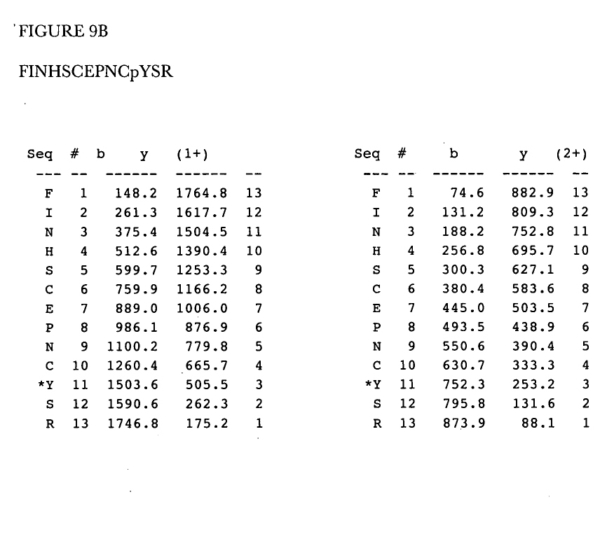

In accordance with the present invention, nearly 219 novel protein phosphorylation sites in signaling pathways underlying NPM-ALK positive Anaplastic Large Cell Lymphoma (ALCL) oncogenesis have now been discovered. These newly described phosphorylation sites were identified by employing the techniques described in “Immunoaffinity Isolation of Modified Peptides From Complex Mixtures,” U.S. Patent Publication No. 20030044848, Rush et al., using cellular extracts from two recognized ALCL cell lines, as further described below. The novel phosphorylation sites, and their corresponding parent proteins, disclosed herein are listed in Table I. These phosphorylation sites correspond to numerous different parent proteins (the full sequences of which (human) are all publicly available in SwissProt database and their Accession numbers listed in Column B of Table 1/FIG. 2), each of which fall into discrete protein type groups, for example Acetyltransferases, Helicases, Kinases, and Transcription Factors (see Column C of Table 1), the phosphorylation of which is relevant to signal transduction activity in ALCL as disclosed herein.

The discovery of the nearly 219 novel protein phosphorylation sites described herein enables the production, by standard methods, of new reagents, such as phosphorylation site-specific antibodies and AQUA peptides (heavy-isotope labeled peptides), capable of specifically detecting and/or quantifying these phosphorylated sites/proteins. Such reagents are highly useful, inter alia, for studying signal transduction events underlying the progression of ALCL. Accordingly, the invention provides novel reagents—phospho-specific antibodies and AQUA peptides—for the specific detection and/or quantification of an ALCL-related signaling protein/polypeptide only when phosphorylated (or only when not phosphorylated) at a particular phosphorylation site disclosed herein. The invention also provides methods of detecting and/or quantifying one or more phosphorylated ALCL-related signaling proteins using the phosphorylation-site specific antibodies and AQUA peptides of the invention, and methods of obtaining a phosphorylation profile of such proteins (e.g. Kinases).

In part, the invention provides an isolated phosphorylation site-specific antibody that specifically binds a given ALCL-related signaling protein only when phosphorylated (or not phosphorylated, respectively) at a particular amino acid enumerated in Column D of Table 1/FIG. 2 comprised within the phosphorylatable peptide site sequence enumerated in corresponding Column E. In further part, the invention provides a heavy-isotope labeled peptide (AQUA peptide) for the quantification of a given ALCL-related signaling protein, the labeled peptide comprising a particular phosphorylatable peptide site/sequence enumerated in Column E of Table 1/FIG. 2 herein. For example, among the reagents provided by the invention is an isolated phosphorylation site-specific antibody that specifically binds the MAPK6 protein only when phosphorylated (or only when not phosphorylated) at tyrosine 628 (see Row 100 (and Columns D and E) of Table 1/FIG. 2). By way of further example, among the group of reagents provided by the invention is an AQUA peptide for the quantification of phosphorylated MAPK6 protein, the AQUA peptide comprising the phosphorylatable peptide sequence listed in Column E, Row 100, of Table 1/FIG. 2.

In one embodiment, the invention provides an isolated phosphorylation site-specific antibody that specifically binds an Anaplastic Large Cell Lymphoma (ALCL)-related signaling protein selected from Column A of Table 1 only when phosphorylated at the tyrosine listed in corresponding Column D of Table 1, comprised within the peptide sequence listed in corresponding Column E of Table 1 (SEQ ID NOs: 1-15, 17-39, 41-48, 50-64, 66-107, 109-148, 151-191, 193-215, 217-219), wherein said antibody does not bind said signaling protein when not phosphorylated at said tyrosine. In another embodiment, the invention provides an isolated phosphorylation site-specific antibody that specifically binds an ALCL-related signaling protein selected from Column A of Table 1 only when not phosphorylated at the tyrosine listed in corresponding Column D of Table 1, comprised within the peptide sequence listed in corresponding Column E of Table 1 (SEQ ID NOs: 1-15, 17-39, 41-48, 50-64, 66-107, 109-148, 151-191, 193-215, 217-219), wherein said antibody does not bind said signaling protein when phosphorylated at said tyrosine. Such reagents enable the specific detection of phosphorylation (or non-phosphorylation) of a novel phosphorylatable site disclosed herein. The invention further provides immortalized cell lines producing such antibodies. In one preferred embodiment, the immortalized cell line is a rabbit or mouse hybridoma.

In another embodiment, the invention provides a heavy-isotope labeled peptide (AQUA peptide) for the quantification of an ALCL-related signaling protein selected from Column A of Table 1, said labeled peptide comprising the phosphorylatable peptide sequence listed in corresponding Column E of Table 1 (SEQ ID NOs: 1-15, 17-39, 41-48, 50-64, 66-107, 109-148, 151-191, 193-215, 217-219), which sequence comprises the phosphorylatable tyrosine listed in corresponding Column D of Table 1. In certain preferred embodiments, the phosphorylatable tyrosine within the labeled peptide is phosphorylated, while in other preferred embodiments, the phosphorylatable tyrosine within the labeled peptide is not phosphorylated.

Reagents (antibodies and AQUA peptides) provided by the invention may conveniently be grouped by the type of ALCL-related signaling protein in which a given phosphorylation site (for which reagents are provided) occurs. The protein types for each respective protein (in which a phosphorylation site has been discovered) are provided in Column C of Table 1/FIG. 2, and include: Acetyltransferases, Actin Binding Proteins, Adaptor/Scaffold Proteins, Adhesion Proteins, Cell Cycle Regulation Proteins, Cell Surface Proteins, Channel Proteins, Chaperone Proteins, Chemokine Proteins, Cytokines, Cytoskeletal Proteins, DNA Binding Proteins, DNA Repair Proteins, Cellular Metabolism and Miscellaneous Enzymes, GTPase Activating Proteins, Guanine Nucleotide Exchange Factors, Helicases, Hydrolases, Inhibitor Proteins, Kinase Proteins, Ligase Proteins, Lipid Binding Proteins, Mitochondrial Proteins, Motor Proteins, Oxidoreductases, Phosphatases, Proteases, Receptor Proteins, RNA Binding Proteins, Secreted Proteins, Transcription Factors, Translation Initiation Complexes, Transcription coactivator/corepressor Proteins, Transferase Proteins, Transporter Proteins, Tumor Suppressor Proteins, Ubiquitin Conjugating System Proteins, and Vesicle Proteins. Each of these distinct protein groups is considered a preferred subset of ALCL-related signal transduction protein phosphorylation sites disclosed herein, and reagents for their detection/quantification may be considered a preferred subset of reagents provided by the invention.

Particularly preferred subsets of the phosphorylation sites (and their corresponding proteins) disclosed herein are those occurring on the following protein types/groups listed in Column C of Table 1/FIG. 2: Protein Kinase Proteins, Adaptor/Scaffold Protein(s), Phosphatase Proteins, Transcription Factor/Transcription Initiation Complex Proteins, Transferase Proteins, Ubiquitin Conjugating System Proteins, Oxidoreductase Proteins, Receptor Proteins, RNA binding Proteins, and Enzymes. Accordingly, among preferred subsets of reagents provided by the invention are isolated antibodies and AQUA peptides useful for the detection and/or quantification of the foregoing preferred protein/phosphorylation site subsets.

In one subset of preferred embodiments, there is provided:

(i) An isolated phosphorylation site-specific antibody that specifically binds a Kinase Protein selected from Column A, Rows 89-109, of Table 1 only when phosphorylated at the tyrosine listed in corresponding Column D, Rows 89-109, of Table 1, comprised within the phosphorylatable peptide sequence listed in corresponding Column E, Rows 89-109, of Table 1 (SEQ ID NOs: 89-107, 109), wherein said antibody does not bind said protein when not phosphorylated at said tyrosine.
(ii) An equivalent antibody to (i) above that only binds the Protein Kinase when not phosphorylated at the disclosed site (and does not bind the protein when it is phosphorylated at the site).
(iii) A heavy-isotope labeled peptide (AQUA peptide) for the quantification of a Protein Kinase selected from Column A, Rows 89-109, said labeled peptide comprising the phosphorylatable peptide sequence listed in corresponding Column E, Rows 89-109, of Table 1 (SEQ ID NOs: 89-107, 109), which sequence comprises the phosphorylatable tyrosine listed in corresponding Column D, Rows 89-109, of Table 1.

Among this preferred subset of reagents, antibodies and AQUA peptides for the detection/quantification of the following Kinase Protein phosphorylation sites are particularly preferred: CSNK2B (Y108), PCTK3 (Y155), BMX (Y197), and ACS (Y524), (see SEQ ID NOs: 97, 101, 109 and 110).

In another subset of preferred embodiments, there is provided:

(i) An isolated phosphorylation site-specific antibody that specifically binds an Adaptor/Scaffold Protein selected from Column A, Rows 3-23, of Table 1 only when phosphorylated at the tyrosine listed in corresponding Column D, Rows 3-23, of Table 1, comprised within the phosphorylatable peptide sequence listed in corresponding Column E, Rows 3-23, of Table 1 (SEQ ID NOs: 3-15, 17-23), wherein said antibody does not bind said protein when not phosphorylated at said tyrosine.
(ii) An equivalent antibody to (i) above that only binds the Adaptor/Scaffold Protein when not phosphorylated at the disclosed site (and does not bind the protein when it is phosphorylated at the site).
(iii) A heavy-isotope labeled peptide (AQUA peptide) for the quantification of an Adaptor/Scaffold Protein selected from Column A, Rows 3-23, said labeled peptide comprising the phosphorylatable peptide sequence listed in corresponding Column E, Rows 3-23, of Table 1 (SEQ ID NOs: 3-15, 17-23), which sequence comprises the phosphorylatable tyrosine listed in corresponding Column D, Rows 3-23, of Table 1.

Among this preferred subset of reagents, antibodies and AQUA peptides for the detection/quantification of the following Adaptor/Scaffold Protein phosphorylation sites are particularly preferred: CBL (Y114), GAB3 (Y542) and IRS4 (Y112), (see SEQ ID NOs: 8, 10, and 13).

In another subset of preferred embodiments, there is provided:

(i) An isolated phosphorylation site-specific antibody that specifically binds a Phosphatase Protein selected from Column A, Rows 125-129, of Table 1 only when phosphorylated at the tyrosine listed in corresponding Column D, Rows 125-129, of Table 1, comprised within the phosphorylatable peptide sequence listed in corresponding Column E, Rows 125-129, of Table 1 (SEQ ID NOs: 125-129), wherein said antibody does not bind said protein when not phosphorylated at said tyrosine.
(ii) An equivalent antibody to (i) above that only binds the Phosphatase Protein when not phosphorylated at the disclosed site (and does not bind the protein when it is phosphorylated at the site).
(iii) A heavy-isotope labeled peptide (AQUA peptide) for the quantification of a Phosphatase Protein selected from Column A, Rows 125-129, said labeled peptide comprising the phosphorylatable peptide sequence listed in corresponding Column E, Rows 125-129, of Table 1 (SEQ ID NOs: 125-129), which sequence comprises the phosphorylatable tyrosine listed in corresponding Column D, Rows 125-129, of Table 1.

Among this preferred subset of reagents, antibodies and AQUA peptides for the detection/quantification of the following Phosphatase Protein phosphorylation sites are particularly preferred: PPP2CB (Y284), and PTPN11 (Y66), (see SEQ ID NOs: 127 and 128).

In another subset of preferred embodiments, there is provided:

(i) An isolated phosphorylation site-specific antibody that specifically binds a Transcription Factor/Transcription Initiation Complex Protein selected from Column A, Rows 167-189, of Table 1 only when phosphorylated at the tyrosine listed in corresponding Column D, Rows 167-189 of Table 1, comprised within the phosphorylatable peptide sequence listed in corresponding Column E, Rows 167-189, of Table 1 (SEQ ID NOs: 167-189), wherein said antibody does not bind said protein when not phosphorylated at said tyrosine.
(ii) An equivalent antibody to (i) above that only binds the Transcription Factor/Transcription Initiation Complex Protein when not phosphorylated at the disclosed site (and does not bind the protein when it is phosphorylated at the site).
(iii) A heavy-isotope labeled peptide (AQUA peptide) for the quantification of a Transcription Factor/Transcription Initiation Complex Protein selected from Column A, Rows 167-189, said labeled peptide comprising the phosphorylatable peptide sequence listed in corresponding Column E, Rows 167-189, of Table 1 (SEQ ID NOs: 167-189), which sequence comprises the phosphorylatable tyrosine listed in corresponding Column D, Rows 167-189, of Table 1.

Among this preferred subset of reagents, antibodies and AQUA peptides for the detection/quantification of the following Transcription Factor/Transcription Initiation Complex Protein phosphorylation sites are particularly preferred: GATA6 (Y417), MLL (Y3914), POLR2A (Y1881) and TP53BP2 (Y487), (see SEQ ID NOs: 168, 170, 180, and 188).

In another subset of preferred embodiments, there is provided:

(i) An isolated phosphorylation site-specific antibody that specifically binds a Transferase Protein selected from Column A, Rows 190-202, of Table 1 only when phosphorylated at the tyrosine listed in corresponding Column D, Rows 190-202, of Table 1, comprised within the phosphorylatable peptide sequence listed in corresponding Column E, Rows 190-202, of Table 1 (SEQ ID NOs: 190-191 and 193-202), wherein said antibody does not bind said protein when not phosphorylated at said tyrosine.
(ii) An equivalent antibody to (i) above that only binds the Transferase Protein when not phosphorylated at the disclosed site (and does not bind the protein when it is phosphorylated at the site).
(iii) A heavy-isotope labeled peptide (AQUA peptide) for the quantification of a Transferase Protein selected from Column A, Rows 190-202, said labeled peptide comprising the phosphorylatable peptide sequence listed in corresponding Column E, Rows 190-202, of Table 1 (SEQ ID NOs: 190-191 and 193-202), which sequence comprises the phosphorylatable tyrosine listed in corresponding Column D, Rows 190-202, of Table 1.

Among this preferred subset of reagents, antibodies and AQUA peptides for the detection/quantification of the following Transferase Protein phosphorylation sites are particularly preferred: ATIC (Y192), (see SEQ ID NO: 191).

In another subset of preferred embodiments, there is provided:

(i) An isolated phosphorylation site-specific antibody that specifically binds an Ubiquitin Conjugating System Protein selected from Column A, Rows 211-215, of Table 1 only when phosphorylated at the tyrosine listed in corresponding Column D, Rows 211-215, of Table 1, comprised within the phosphorylatable peptide sequence listed in corresponding Column E, Rows 211-215, of Table 1 (SEQ ID NOs: 211-215), wherein said antibody does not bind said protein when not phosphorylated at said tyrosine.
(ii) An equivalent antibody to (i) above that only binds the Ubiquitin Conjugating System Protein when not phosphorylated at the disclosed site (and does not bind the protein when it is phosphorylated at the site).
(iii) A heavy-isotope labeled peptide (AQUA peptide) for the quantification of an Ubiquitin Conjugating System Protein selected from Column A, Rows 211-215, said labeled peptide comprising the phosphorylatable peptide sequence listed in corresponding Column E, Rows 211-215, of Table 1 (SEQ ID NOs: 211-215), which sequence comprises the phosphorylatable tyrosine listed in corresponding Column D, Rows 211-215, of Table 1.

Among this preferred subset of reagents, antibodies and AQUA peptides for the detection/quantification of the following Ubiquitin Conjugating System Protein phosphorylation sites are particularly preferred: DTX3L (Y235) and USP11 (Y870), (see SEQ ID NOs: 212 and 214).

In another subset of preferred embodiments, there is provided:

(i) An isolated phosphorylation site-specific antibody that specifically binds an Oxidoreductase Protein selected from Column A, Rows 117-124, of Table 1 only when phosphorylated at the tyrosine listed in corresponding Column D, Rows 117-124, of Table 1, comprised within the phosphorylatable peptide sequence listed in corresponding Column E, Rows 117-124, of Table 1 (SEQ ID NOs: 117-124), wherein said antibody does not bind said protein when not phosphorylated at said tyrosine.
(ii) An equivalent antibody to (i) above that only binds the Oxidoreductase Protein when not phosphorylated at the disclosed site (and does not bind the protein when it is phosphorylated at the site).
(iii) A heavy-isotope labeled peptide (AQUA peptide) for the quantification of an Oxidoreductase Protein selected from Column A, Rows 117-124, said labeled peptide comprising the phosphorylatable peptide sequence listed in corresponding Column E, Rows 117-124, of Table 1 (SEQ ID NOs: 117-124), which sequence comprises the phosphorylatable tyrosine listed in corresponding Column D, Rows 117-124, of Table 1.

Among this preferred subset of reagents, antibodies and AQUA peptides for the detection/quantification of the following Oxidoreductase Protein phosphorylation site is particularly preferred: GSR (Y67) (see SEQ ID NO: 117).

In another subset of preferred embodiments, there is provided:

(i) An isolated phosphorylation site-specific antibody that specifically binds a Receptor Protein selected from Column A, Rows 142-150, of Table 1 only when phosphorylated at the tyrosine listed in corresponding Column D, Rows 142-150, of Table 1, comprised within the phosphorylatable peptide sequence listed in corresponding Column E, Rows 142-150, of Table 1 (SEQ ID NOs: 142-148), wherein said antibody does not bind said protein when not phosphorylated at said tyrosine.
(ii) An equivalent antibody to (i) above that only binds the Receptor Protein when not phosphorylated at the disclosed site (and does not bind the protein when it is phosphorylated at the site).
(iii) A heavy-isotope labeled peptide (AQUA peptide) for the quantification of a Receptor Protein selected from Column A, Rows 142-150, said labeled peptide comprising the phosphorylatable peptide sequence listed in corresponding Column E, Rows 142-150, of Table 1 (SEQ ID NOs: 142-148), which sequence comprises the phosphorylatable tyrosine listed in corresponding Column D, Rows 142-150, of Table 1.

Among this preferred subset of reagents, antibodies and AQUA peptides for the detection/quantification of the following Receptor Protein phosphorylation site is particularly preferred: ADRA2B (Y120) (see SEQ ID NO: 142).

In another subset of preferred embodiments, there is provided:

(i) An isolated phosphorylation site-specific antibody that specifically binds a RNA Binding Protein selected from Column A, Rows 151-162, of Table 1 only when phosphorylated at the tyrosine listed in corresponding Column D, Rows 151-162, of Table 1, comprised within the phosphorylatable peptide sequence listed in corresponding Column E, Rows 151-162, of Table 1 (SEQ ID NOs: 151-162), wherein said antibody does not bind said protein when not phosphorylated at said tyrosine.
(ii) An equivalent antibody to (i) above that only binds the RNA Binding Protein when not phosphorylated at the disclosed site (and does not bind the protein when it is phosphorylated at the site).
(iii) A heavy-isotope labeled peptide (AQUA peptide) for the quantification of a RNA Binding Protein selected from Column A, Rows 151-162, said labeled peptide comprising the phosphorylatable peptide sequence listed in corresponding Column E, Rows 151-162, of Table 1 (SEQ ID NOs: 151-162), which sequence comprises the phosphorylatable tyrosine listed in corresponding Column D, Rows 151-162, of Table 1.

Among this preferred subset of reagents, antibodies and AQUA peptides for the detection/quantification of the following RNA Binding Protein phosphorylation site is particularly preferred: NUP160 (Y355) (see SEQ ID NO: 156).

In another subset of preferred embodiments, there is provided:

(i) An isolated phosphorylation site-specific antibody that specifically binds an Enzyme selected from Column A, Rows 62-76, of Table 1 only when phosphorylated at the tyrosine listed in corresponding Column D, Rows 62-76, of Table 1, comprised within the phosphorylatable peptide sequence listed in corresponding Column E, Rows 62-76, of Table 1 (SEQ ID NOs: 62-64: 66-76), wherein said antibody does not bind said protein when not phosphorylated at said tyrosine.
(ii) An equivalent antibody to (i) above that only binds the Enzyme when not phosphorylated at the disclosed site (and does not bind the protein when it is phosphorylated at the site).
(iii) A heavy-isotope labeled peptide (AQUA peptide) for the quantification of an Enzyme selected from Column A, Rows 62-76, said labeled peptide comprising the phosphorylatable peptide sequence listed in corresponding Column E, Rows 62-76, of Table 1 (SEQ ID NOs: 62-64, 66-76), which sequence comprises the phosphorylatable tyrosine listed in corresponding Column D, Rows 62-76, of Table 1.

Among this preferred subset of reagents, antibodies and AQUA peptides for the detection/quantification of the following Enzyme phosphorylation sites are particularly preferred: ACLY (Y588) (see SEQ ID NO: 76).

In another subset of preferred embodiments, there is provided:

(i) An isolated phosphorylation site-specific antibody that specifically binds a protein selected from Column A, Rows 32, 78, 79 and 211, of Table 1 only when phosphorylated at the tyrosine listed in corresponding Column D, Rows 32, 78, 79 and 211, of Table 1, comprised within the phosphorylatable peptide sequence listed in corresponding Column E, Rows 32, 78, 79 and 211, of Table 1 (SEQ ID NOs: 32, 78, 79 and 211), wherein said antibody does not bind said protein when not phosphorylated at said tyrosine.
(ii) An equivalent antibody to (i) above that only binds the protein when not phosphorylated at the disclosed site (and does not bind the protein when it is phosphorylated at the site).
(iii) A heavy-isotope labeled peptide (AQUA peptide) for the quantification of a protein selected from Column A, Rows 32, 78, 79 and 211, said labeled peptide comprising the phosphorylatable peptide sequence listed in corresponding Column E, Rows 32, 78, 79 and 211, of Table 1 (SEQ ID NOs: 32, 78, 79 and 211), which sequence comprises the phosphorylatable tyrosine listed in corresponding Column D, Rows 32, 78, 79 and 211, of Table 1.

The invention also provides, in part, an immortalized cell line producing an antibody of the invention, for example, a cell line producing an antibody within any of the foregoing preferred subsets of antibodies. In one preferred embodiment, the immortalized cell line is a rabbit hybridoma or a mouse hybridoma.

In certain other preferred embodiments, a heavy-isotope labeled peptide (AQUA peptide) of the invention (for example, an AQUA peptide within an of the foregoing preferred subsets of AQUA peptides) comprises a disclosed site sequence wherein the phosphorylatable tyrosine is phosphorylated. In certain other preferred embodiments, a heavy-isotope labeled peptide of the invention comprises a disclosed site sequence wherein the phosphorylatable tyrosine is not phosphorylated.

The foregoing subsets of preferred reagents of the invention should not be construed as limiting the scope of the invention, which, as noted above, includes reagents for the detection and/or quantification of disclosed phosphorylation sites on any of the other protein type/group subsets (each a preferred subset) listed in Column C of Table 1/FIG. 2.

Also provided by the invention are methods for detecting or quantifying a signaling protein that is tyrosine-phosphorylated in human Anaplastic Large Cell Lymphoma (ALCL), said method comprising the step of utilizing one or more of the above-described reagents of the invention to detect or quantify one or more ALCL-related signaling protein(s) selected from Column A of Table 1 only when phosphorylated at the tyrosine listed in corresponding Column D of Table 1. In certain preferred embodiments of the methods of the invention, the reagents comprise a subset of preferred reagents as described above.

Also provided by the invention is a method for obtaining a phosphorylation profile of protein kinases that are phosphorylated in Carcinoma signaling pathways, said method comprising the step of utilizing one or more isolated antibody that specifically binds a protein kinase selected from Column A, Rows 138-165, of Table 1 only when phosphorylated at the tyrosine listed in corresponding Column D, Rows 138-165, of Table 1, comprised within the phosphorylation site sequence listed in corresponding Column E, Rows 138-165, of Table 1 (SEQ ID NOs: 137-154, and 156-164), to detect the phosphorylation of one or more of said protein kinases, thereby obtaining a phosphorylation profile for said kinases.

The identification of the disclosed novel ALCL-related phosphorylation sites, and the standard production and use of the reagents provided by the invention are described in further detail below and in the Examples that follow.

All cited references are hereby incorporated herein, in their entirety, by reference. The Examples are provided to further illustrate the invention, and do not in any way limit its scope, except as provided in the claims appended hereto.

TABLE 1
Newly-Discovered ALCL-Related Phosphorylation Sites.

| | | | | | |
|---|---|---|---|---|---|
| A | | | D | | H |
| Gene | B | C | Phospho- | E | SEQ ID |
| Symbol | Accession No. | Protein Type | Residue | Phosphorylation Site Sequence | NO |

| | | | | | | |
|---|---|---|---|---|---|---|
| 1 | PCAF | NP_003875.3 | Acetyltransferase | Y729 | DPDQLySTLK | SEQ ID |
| | | | | | | NO: 1 |
| 2 | CMYA1 | NP_919269.2 | Actin binding | Y1143 | GLPGGWVTIQDGIyTAHPVR | SEQ ID |
| | | | protein | | | NO: 2 |
| 3 | AKAP9 | NP_005742.4 | Adaptor/scafffold | Y3839 | SRSDLDyIR | SEQ ID |
| | | | | | | NO: 3 |
| 4 | APBB1IP | NP_061916.3 | Adaptor/scaffold | Y374 | yKAPTDYCFVLKHPQIQK | SEQ ID |
| | | | | | | NO: 4 |
| 5 | APBB1IP | NP_061916.3 | Adaptor/scaffold | Y380 | APTDyCFVLK | SEQ ID |
| | | | | | | NO: 5 |
| 6 | BRDG1 | NP_036240.1 | Adaptor/scaffold | Y65 | TDKKSIIyVDKLDIV | SEQ ID |
| | | | | | | NO: 6 |
| 7 | CBL | NP_005179.2 | Adaptor/scaffold | Y102 | yEGKMETLGENEYFR | SEQ ID |
| | | | | | | NO: 7 |
| 8 | CBL | NP_005179.2 | Adaptor/scaffold | Y114 | YEGKMETLGENEyFR | SEQ ID |
| | | | | | | NO: 8 |
| 9 | GAB3 | NP_542179.1 | Adaptor/scaffold | Y395 | SASIEDSyVPMSPQA | SEQ ID |
| | | | | | | NO: 9 |
| 10 | GAB3 | NP_542179.1 | Adaptor/scaffold | Y542 | FSLDyLALDFNSASPAPMQQK | SEQ ID |
| | | | | | | NO: 10 |
| 11 | GPSM2 | NP_037428.2 | Adaptor/scaffold | Y139 | ALyNLGNVYHAK | SEQ ID |
| | | | | | | NO: 11 |
| 12 | IRS4 | NP_003595.1 | Adaptor/scaffold | Y111 | LETADAPARLEyYENAR | SEQ ID |
| | | | | | | NO: 12 |
| 13 | IRS4 | NP_003595.1 | Adaptor/scaffold | Y112 | LETADAPARLEYyENAR | SEQ ID |
| | | | | | | NO: 13 |
| 14 | LCP2 | NP_005556.1 | Adaptor/scaffold | Y459 | TTNPyVLMVLYK | SEQ ID |
| | | | | | | NO: 14 |
| 15 | LCP2 | NP_005556.1 | Adaptor/scaffold | Y465 | PYVLMVLyKDKVYNI | SEQ ID |
| | | | | | | NO: 15 |
| 16 | RANBP2 | | Adaptor/scaffold | Y1349 | IVKKEGPyWNCNSCS | SEQ ID |
| | | | | | | NO: 16 |
| 17 | RANBP2 | NP_006258.2 | Adaptor/scaffold | Y70 | FLGLLyELEENTDK | SEQ ID |
| | | | | | | NO: 17 |
| 18 | RAPH1 | NP_079528.1 | Adaptor/scaffold | Y518 | YKAPTDyCLVLK | SEQ ID |
| | | | | | | NO: 18 |
| 19 | SH2D2A | NP_683720.2 | Adaptor/scaffold | Y178 | LQDLLLHYTAHPLSPyGETLTEPLAR | SEQ ID |
| | | | | | | NO: 19 |
| 20 | STRAP | NP_009109.2 | Adaptor/scaffold | Y114 | TVDFTQDSNyLLTGGQDK | SEQ ID |
| | | | | | | NO: 20 |
| 21 | TJP1 | NP_003248.2 | Adaptor/scaffold | Y1354 | SNHYDPEEDEEyYR | SEQ ID |
| | | | | | | NO: 21 |
| 22 | YWHAG | NP_036611.2 | Adaptor/scaffold | Y179 | LGLALNySVFYYEIQNAPEQACHLAK | SEQ ID |
| | | | | | | NO: 22 |
| 23 | CBLB | NP_733762.2 | Adaptor/scaffold; | Y276 | ARLQKySTKPGSYIFR | SEQ ID |
| | | | Calcium-binding | | | NO: 23 |
| | | | protein | | | |
| 24 | CDH18 | NP_004925.1 | Adhesion | Y388 | DATMLKIIVGDVDEPPLFSMPSyLMEVYENAK | SEQ ID |
| | | | | | | NO: 24 |
| 25 | FHL1 | NP_001440.2 | Adhesion | Y207 | FTAVEDQyYCVDCYK | SEQ ID |
| | | | | | | NO: 25 |
| 26 | FLRT3 | NP_938205.1 | Adhesion | Y105 | FPTNLPKyVKELHLQ | SEQ ID |
| | | | | | | NO: 26 |
| 27 | FLRT3 | NP_938205.1 | Adhesion | Y89 | LLKVERIyLYHNSLD | SEQ ID |
| | | | | | | NO: 27 |
| 28 | NRXN3 | NP_004787.2 | Adhesion | Y554 | GyIHYVFDLGNGPNVIK | SEQ ID |
| | | | | | | NO: 28 |
| 29 | DSP | NP_001008844.1 | Adhesion; Cyto- | Y1676 | LGIyEAMK | SEQ ID |
| | | | skeletal protein | | | NO: 29 |
| 30 | DSP | NP_001008844.1 | Adhesion; Cyto- | Y249 | SAIyQLEEEYENLLK | SEQ ID |
| | | | skeletal protein | | | NO: 30 |
| 31 | TAX1BP1 | NP_006015.4 | Apoptosis | Y555 | AKCNKyADELAKMELKWK | SEQ ID |
| | | | | | | NO: 31 |
| 32 | CASP8 | NP_001219.2 | Apoptosis; Protease | Y382 | PKVFFIQACQGDNyQK | SEQ ID |
| | | | (non-proteasomal) | | | NO: 32 |
| 33 | TNNC1 | NP_003271.1 | Calcium-binding | Y111 | NADGyIDLDELK | SEQ ID |
| | | | protein | | | NO: 33 |
| 34 | BRRN1 | NP_056156.2 | Cell cycle | Y428 | TMCPLLSMKPGEySYFSPR | SEQ ID |
| | | | regulation | | | NO: 34 |
| 35 | CNAP1 | NP_055680.2 | Cell cycle | Y1228 | DLAyCVSQLPLTER | SEQ ID |
| | | | regulation | | | NO: 35 |
| 36 | SAS-6 | NP_919268.1 | Cell cycle | Y522 | LTyPTCGIGYPVSSAFAFQNTFPHSISAK | SEQ ID |
| | | | regulation | | | NO: 36 |
| 37 | SEPT7 | NP_001011553.1 | Cell cycle | Y10 | NLEGyVGFANLPNQVYR | SEQ ID |
| | | | regulation | | | NO: 37 |
| 38 | VCP | NP_003966.1 | Cell cycle | Y173 | VVETDPSPyCIVAPDTVIHCEGEPIKR | SEQ ID |
| | | | regulation | | | NO: 38 |
| 39 | CSPG6 | NP_00S436.1 | Cell cycle | Y225 | ALEyTIYNQELNETR | SEQ ID |
| | | | regulation; | | | NO: 39 |
| | | | DNA repair | | | |
| 40 | LY9 | | Cell surface | Y604 | KEDSNTIyCSVQKPK | SEQ ID |
| | | | | | | NO: 40 |
| 41 | VMD2L3 | NP_116124.1 | Cell surface | Y131 | LRRTLMRyVNLTSLL | SEQ ID |
| | | | | | | NO: 41 |
| 42 | VMD2L3 | NP_116124.1 | Cell surface | Y148 | RSVSTAVyKRFPTMD | SEQ ID |
| | | | | | | NO: 42 |
| 43 | TRPC4 | NP_057263.1 | Channel, cation | Y14 | NVNAPyR | SEQ ID |
| | | | | | | NO: 43 |
| 44 | VDAC1 | NP_003365.1 | Channel, misc. | Y67 | WTEyGLTFTEK | SEQ ID |
| | | | | | | NO: 44 |
| 45 | HSPA2 | NP_068814.2 | Chaperone | Y135 | MKEIAEAyLGGKVHSAVITVPAYFNDSQR | SEQ ID |
| | | | | | | NO: 45 |
| 46 | HSPCB | NP_031381.2 | Chaperone | Y485 | SIYyITGESK | SEQ ID |
| | | | | | | NO: 46 |
| 47 | STIP1 | NP_006810.1 | Chaperone | Y248 | KDFDTALKHyDK | SEQ ID |
| | | | | | | NO: 47 |
| 48 | TRAP1 | NP_057376.1 | Chaperone | Y498 | NIyYLCAPNR | SEQ ID |
| | | | | | | NO: 48 |
| 49 | Cxcl15 | | Chemokine | Y119 | QKEFPPAMKLLYSVEHEKPLyLSFGRPENK | SEQ ID |
| | | | | | | NO: 49 |
| 50 | PBEF1 | NP_005737.1 | Cytokine | Y23 | VTHyKQYPPNTSK | SEQ ID |
| | | | | | | NO: 50 |
| 51 | PBEF1 | NP_005737.1 | Cytokine | Y34 | QYPPNTSKVySYFECR | SEQ ID |
| | | | | | | NO: 51 |
| 52 | ACTL6B | NP_057272.1 | Cytoskeletal | Y104 | RAILDHTySKHVKSE | SEQ ID |
| | | | protein | | | NO: 52 |
| 53 | EPPK1 | NP112598.1 | Cytoskelelal | Y4959 | AVTGYTDPyTGQQISLFQAMQK | SEQ ID |
| | | | protein | | | NO: 53 |
| 54 | NEB | NP_004534.1 | Cytoskeletal | Y5021 | VNNVTSERLyRELYHK | SEQ ID |
| | | | protein | | | NO: 54 |
| 55 | ODF3 | NP_444510.2 | Cytoskeletal | Y200 | VTKFKAPQyTMAARVEPPGDKTLK | SEQ ID |
| | | | protein | | | NO: 55 |
| 56 | ARID1B | NP_059989.1 | DNA binding | Y1488 | KRHMDGMyGPPAKRH | SEQ ID |
| | | | protein | | | NO: 56 |
| 57 | PARP4 | NP_006428.1 | DNA binding | Y1459 | GFGSYHPSAySPFHFQPSAASLTANLR | SEQ ID |
| | | | protein | | | NO: 57 |
| 58 | ZNF261 | NP_005087.1 | DNA binding | Y567 | TVyQFCSPSCWTK | SEQ ID |
| | | | protein | | | NO: 58 |
| 59 | DDB1 | NP_001914.2 | DNA repair | Y871 | GAVySMVEFNGK | SEQ ID |
| | | | | | | NO: 59 |
| 60 | RAD18 | NP_064550.2 | DNA repair | Y56 | FLSyKTQCPTCCVTVTEPDLK | SEQ ID |
| | | | | | | NO: 60 |
| 61 | RIF1 | NP_060621.3 | DNA repair | Y1659 | yAEYSFTSLPVPESNLR | SEQ ID |
| | | | | | | NO: 61 |
| 62 | GMDS | NP_001491.1 | Enzyme, cellular | Y323 | DLKyYRPT | SEQ ID |
| | | | metabolism | | | NO: 62 |
| 63 | GMDS | NP_001491.1 | Enzyme, cellular | Y324 | DLKYyRPTEV | SEQ ID |
| | | | metabolism | | | NO: 63 |
| 64 | PFKL | NP_001002021.1 | Enzyme, cellular | Y681 | CHDYyTTEFLYNLYSSEGK | SEQ ID |
| | | | metabolism: Kinase | | | NO: 64 |
| | | | (non-protein) | | | |
| 65 | PKM2 | | Enzyme, cellular | Y466 | HLyRGIFPV | SEQ ID |
| | | | metabolism: | | | NO: 65 |
| | | | Unknown | | | |
| | | | function | | | |
| 66 | CA2 | NP_000058.1 | Enzyme, misc. | Y88 | GGPLDGTyR | SEQ ID |
| | | | | | | NO: 66 |
| 67 | CAD | NP_004332.2 | Enzyme, misc. | Y735 | NSVTGGTAAFEPSVDyCVVKIPR | SEQ ID |
| | | | | | | NO: 67 |
| 68 | CBR1 | NP_001748.1 | Enzyme, misc. | Y194 | EGWPSSAyGVTK | SEQ ID |
| | | | | | | NO: 68 |
| 69 | CBR1 | NP_001748.1 | Enzyme, misc. | Y253 | SPEEGAETPVyLALLPPDAEGPHGQFVSEK | SEQ ID |
| | | | | | | NO: 69 |
| 70 | FARSLB | NP_005678.2 | Enzyme, misc. | Y192 | TKEyTACELMNIYK | SEQ ID |
| | | | | | | NO: 70 |
| 71 | GMPS | NP_003866.1 | Enzyme, misc. | Y526 | SySYVCGISSKDEPDWESLIFLAR | SEQ ID |
| | | | | | | NO: 71 |
| 72 | GYS1 | NP_002094.2 | Enzyme, misc. | Y405 | KLyESLLVGSLPDMNK | SEQ ID |
| | | | | | | NO: 72 |
| 73 | NARG1 | NP_476516.1 | Enzyme, misc. | Y834 | ANCHKLFPyALAFMPPGYEEDMK | SEQ ID |
| | | | | | | NO: 73 |
| 74 | RARS | NP_002878.2 | Enzyme, misc. | Y536 | GNTAAYLLyAFTR | SEQ ID |
| | | | | | | NO: 74 |
| 75 | TARS | NP_689508.3 | Enzyme, misc. | Y333 | DQELyFFHELSPGSCFFLPK | SEQ ID |
| | | | | | | NO: 75 |
| 76 | ACLY | NP_001087.2 | Enzyme, misc.; | Y588 | SAYDSTMETMNyAQIR | SEQ ID |
| | | | Lyase | | | NO: 76 |
| 77 | COL4A2 | NP_001837.1 | Extracellular | Y274 | GDVGQPGPNGIPSDTLHPIIAPTGVTFHPDQyK | SEQ ID |
| | | | matrix | | | NO: 77 |
| 78 | TBC1D1 | NP_055988.2 | GTPase activating | Y625 | SQRKLMRyHSVSTET | SEQ ID |
| | | | protein, misc. | | | NO: 78 |
| 79 | ARHGEF2 | NP_004714.2 | Guanine nucleotide | Y406 | ELLSNVDEGIyQLEK | SEQ ID |
| | | | exchange factor, | | | NO: 79 |
| | | | Rac/Rho | | | |
| 80 | DDX6 | NP_004388.1 | Helicase | Y302 | GVTQYyAYVTER | SEQ ID |
| | | | | | | NO: 80 |
| 81 | DHX9 | NP_001348.2 | Helicase | Y9 | NFLyAWCGKR | SEQ ID |
| | | | | | | NO: 81 |
| 82 | DICER1 | NP_085124.2 | Helicase | Y1204 | DFCQGNQLNyYK | SEQ ID |
| | | | | | | NO: 82 |
| 83 | HELZ | NP_055692.2 | Helicase | Y1573 | LTSSAEDEVETTySR | SEQ ID |
| | | | | | | NO: 83 |
| 84 | ESD | NP_001975.1 | Hydrolase, esterase | Y262 | LQEGYDHSyYFIATFITDHIR | SEQ ID |
| | | | | | | NO: 84 |
| 85 | ESD | NP_001975.1 | Hydrolase, esterase | Y263 | LQEGYDHSYyFIATFITDHIR | SEQ ID |
| | | | | | | NO: 85 |
| 86 | COPS3 | NP_003644.2 | Inhibitor protein | Y227 | MLESYKKyILVSLIL | SEQ ID |
| | | | | | | NO: 86 |
| 87 | CRHBP | NP_001873.2 | Inhibitor protein | Y298 | VTFEyRQLEPYELENPNGNSIGEFCLSGL | SEQ ID |
| | | | | | | NO: 87 |
| 88 | TANK | NP_004171.2 | Inhibitor protein | Y73 | SQLLLVNSTQDNNyGCVPLLEDSETR | SEQ ID |
| | | | | | | NO: 88 |
| 89 | GUK1 | NP_000849.1 | Kinase (non-protein) | Y53 | NPRPGEENGKDyYFVTR | SEQ ID |
| | | | | | | NO: 89 |
| 90 | DGKB | NP_004071.1 | Kinase, lipid | Y586 | DPVPYSIINNyF | SEQ ID |
| | | | | | | NO: 90 |
| 91 | DGKI | NP_004708.1 | Kinase, lipid | Y400 | PLLVFVNPKSGGNQGTKVLQMFMWyLNPR | SEQ ID |
| | | | | | | NO: 91 |
| 92 | PIP5K2B | NP_003550.1 | Kinase, lipid | Y98 | FKEyCPMVFR | SEQ ID |
| | | | | | | NO: 92 |
| 93 | PIK3C2A | NP_002636.1 | KINASE; Kinase, | Y1595 | DLVTEDGADPNPyVK | SEQ ID |
| | | | lipid | | | NO: 93 |
| 94 | CLK1 | NP_004062.2 | KINASE; Protein | Y460 | MLEyDPAKRITLR | SEQ ID |
| | | | kinase, dual- | | | NO: 94 |
| | | | specificity | | | |
| 95 | TTK | NP_003309.2 | KINASE; Protein | Y462 | TPSSNTLDDyMSCFR | SEQ ID |
| | | | kinase, dual- | | | NO: 95 |
| | | | specificity | | | |
| 96 | CAMK1 | NP_003647.1 | KINASE; Protein | Y184 | TACGTPGyVAPEVLA | SEQ ID |
| | | | kinase, Ser/Thr | | | NO: 96 |
| | | | (non-receptor) | | | |
| 97 | CSNK2B | NP_001311.3 | KINASE; Protein | Y108 | YQQGDFGyCPR | SEQ ID |
| | | | kinase, Ser/Thr | | | NO: 97 |
| | | | (non-receptor) | | | |
| 98 | GAK | NP_005246.1 | KINASE; Protein | Y153 | IFyQTCRAVQHMHRQK | SEQ ID |
| | | | kinase, Ser/Thr | | | NO: 98 |
| | | | (non-receptor) | | | |
| 99 | KIAA2002 | XP_940171.1 | KINASE; Protein | Y462 | ASTDVAGQAVTINLVPTEEQAKPyR | SEQ ID |
| | | | kinase, Ser/Thr | | | NO: 99 |
| | | | (non-receptor) | | | |
| 100 | MAPK6 | NP_002739.1 | KINASE; Protein | Y628 | KDEQVEKENTYTSyLDK | SEQ ID |
| | | | kinase, Ser/Thr | | | NO: 100 |
| | | | (non-receptor) | | | |
| 101 | PCTK3 | NP_002587.2 | KINASE; Protein | Y155 | LGEGTyATVFKGR | SEQ ID |
| | | | kinase, Ser/Thr | | | NO: 101 |
| | | | (non-receptor) | | | |
| 102 | PRKAA1 | NP_006242.5 | KINASE; Protein | Y424 | QLDyEWKVVNPYYLR | SEQ ID |
| | | | kinase, Ser/Thr | | | NO: 102 |
| | | | (non-receptor) | | | |
| 103 | PRKAA1 | NP_006242.5 | KINASE; Protein | Y532 | QLDYEWKVVNPyYLR | SEQ ID |
| | | | kinase, Ser/Thr | | | NO: 103 |
| | | | (non-receptor) | | | |
| 104 | PRKAA1 | NP_006242.5 | KINASE; Protein | Y433 | QLDYEWKVVNPYyLR | SEQ ID |
| | | | kinase, Ser/Thr | | | NO: 104 |
| | | | (non-receptor) | | | |
| 105 | PRPF4B | NP_003904.3 | KINASE; Protein | Y674 | DNWTDAEGyYR | SEQ ID |
| | | | kinase, Ser/Thr | | | NO: 105 |
| | | | (non-receptor) | | | |
| 106 | RNASEL | NP_066956.1 | KINASE; Protein | Y691 | MKLKIGDPSLyFQK | SEQ ID |
| | | | kinase, Ser/Thr | | | NO: 106 |
| | | | (non-receptor) | | | |
| 107 | BMX | NP_975010.1 | KINASE; Protein | Y194 | SSTTLAQyDNESKKN | SEQ ID |
| | | | kinase, tyrosine | | | NO: 107 |
| | | | (non-receptor) | | | |
| 108 | BMX | | KINASE Protein | Y197 | ILPQYDSySKKSCGS | SEQ ID |
| | | | kinase tyrosine | | | NO: 108 |
| | | | (non-receptor) | | | |
| 109 | ALK | NP_004295.2 | KINASE; Receptor | Y1283 | DIYRASYyRK | SEQ ID |
| | | | tyrosine kinase | | | NO: 109 |
| 110 | AACS | NP_076417.2 | Ligase | Y524 | FPGIWAHGDyCR | SEQ ID |
| | | | | | | NO: 110 |
| 111 | HDLBP | NP_005237.1 | Lipid binding | Y738 | DIRAKPEyHKFLIGK | SEQ ID |
| | | | protein; RNA | | | NO: 111 |
| | | | binding protein; | | | |
| | | | Transporter, | | | |
| | | | facilitator | | | |
| 112 | SSBP1 | NP_003134.1 | Mitochondrial | Y99 | DVAyQYVK | SEQ ID |
| | | | | | | NO: 112 |
| 113 | TXNRD2 | NP_006431.2 | Mitochondrial | Y40 | GAAAGQRDyDLLVVGGGSGGLACAK | SEQ ID |
| | | | | | | NO: 113 |
| 114 | DCTN2 | NP_006391.1 | Motor protein | Y91 | TGYESGEyEMLGEGLGVK | SEQ ID |
| | | | | | | NO: 114 |
| 115 | KLC2 | NP_073733.1 | Motor protein | Y345 | AEEVEyYYR | SEQ ID |
| | | | | | | NO: 115 |
| 116 | TPM3 | NP_689476.1 | Motor protein; | Y220 | DDLEDELyAQKLKYK | SEQ ID |
| | | | Actin binding | | | NO: 116 |
| | | | protein | | | |
| 117 | GSR | NP_000628.2 | Oxidoreductase | Y67 | QEPQPQGPPPAAGAVASYDyLVIGGGSGGLASAR | SEQ ID |
| | | | | | | NO: 117 |
| 118 | LOX | NP_002308.2 | Oxidoreductase | Y396 | VVRCDIRyTGHHAYA | SEQ ID |
| | | | | | | NO: 118 |
| 119 | LOX | NP_002308.2 | Oxidoreductase | Y402 | RYTGHHAyASGCTIS | SEQ ID |
| | | | | | | NO: 119 |
| 120 | LOX | NP_002308.2 | Oxidoreductase | Y411 | SGCTISPy | SEQ ID |
| | | | | | | NO: 120 |
| 121 | MDH2 | NP_005909.2 | Oxidoreductase | Y161 | KHGVyNPNKIFGVTTLDIVR | SEQ ID |
| | | | | | | NO: 121 |
| 122 | NOS1 | NP_000611.1 | Oxidoreductase | Y593 | WYGLPAVSNMLLEIGGLEFSACPFSGWyMGTEIGVR | SEQ ID |
| | | | | | | NO: 122 |
| 123 | OGDH | NP_001003941.1 | Oxidoreductase | Y304 | TIIDKSSENGVDyVIMGMPHR | SEQ ID |
| | | | | | | NO: 123 |
| 124 | RRM1 | NP_001024.1 | Oxidoreductase | Y485 | IIDINyYPVPEACLSNKR | SEQ ID |
| | | | | | | NO: 124 |
| 125 | PHPT1 | NP_054891.2 | Phosphatase | Y113 | AKyPDYEVTWANDGY | SEQ ID |
| | | | | | | NO: 125 |
| 126 | ITPA | NP_258412.1 | Phosphatase | Y113 | LKPEGLHQLLAGFEDKSAyALCTFALSTGDPSQPVR | SEQ ID |
| | | | (non-protein) | | | NO: 126 |
| 127 | PPP2CB | NP_001009552.1 | PHOSPHATASE; | Y284 | CGNQAAIMELDDTLKySFLQFDPAPR | SEQ ID |
| | | | Protein | | | NO: 127 |
| | | | phosphatase, | | | |
| | | | Ser/Thr (non- | | | |
| | | | receptor) | | | |
| 128 | PTPN11 | NP_002825.3 | PHOSPHATASE; | Y66 | IQNTGDYYDLyGGEK | SEQ ID |
| | | | Protein | | | NO: 128 |
| | | | phosphatase, | | | |
| | | | tyrosine (non- | | | |
| | | | receptor) | | | |
| 129 | PTPRC | NP_002829.2 | PHOSPHATASE; | Y1015 | yINASFIMSYWKPEVMIAAQGPLK | SEQ ID |
| | | | Receptor | | | NO: 129 |
| | | | protein | | | |
| | | | phosphatase, | | | |
| | | | tyrosine | | | |
| 130 | PLA2G4B | NP_005081.1 | Phospholipase | Y939 | EySAPGVR | SEQ ID |
| | | | | | | NO: 130 |
| 131 | PLA2G6 | NP_003551.2 | Phospholipase | Y501 | VFRGSRPyESGPLEE | SEQ ID |
| | | | | | | NO: 131 |
| 132 | DPP3 | NP_005691.2 | Protease (non- | Y211 | EVDGEGKPyYEVR | SEQ ID |
| | | | proteasomal) | | | NO: 132 |
| 133 | PMPCB | NP_004270.2 | Protease (non- | Y141 | SQLDLELEIENMGAHLNAYTSREQTVyYAKAFSK | SEQ ID |
| | | | proteasomal) | | | NO: 133 |
| 134 | PSMA2 | NP_002778.1 | Protease | Y97 | KLAQQyYLVYQEPIPTAQLVQR | SEQ ID |
| | | | (proteasomal | | | NO: 134 |
| | | | subunit) | | | |
| 135 | PSMA7 | NP_002783.1 | Protease | Y153 | LYQTDPSGTyHAWK | SEQ ID |
| | | | (proteasomal | | | NO: 135 |
| | | | subunit) | | | |
| 136 | PSMB1 | NP_002784.1 | Protease | Y132 | LYSRRFFPyYVYNIIGGLDEEGKG | SEQ ID |
| | | | (proteasomal | | | NO: 136 |
| | | | subunit) | | | |
| 137 | PSMB5 | NP_002788.1 | Protease | Y149 | LANMVYQyKGMGLSM | SEQ ID |
| | | | (proteasomal | | | NO: 137 |
| | | | subunit) | | | |
| 138 | PSMB8 | NP_004150.1 | Protease | Y180 | DKKGPGLyYVDEHGTRL | SEQ ID |
| | | | (proteasomal | | | NO: 138 |
| | | | subunit) | | | |
| 139 | PSMC4 | NP_006494.1 | Protease | Y112 | AVDQNTAIVGSTTGSNYyVRILSTIDRE | SEQ ID |
| | | | (proteasomal | | | NO: 139 |
| | | | subunit) | | | |
| 140 | PSMD8 | NP_002803.1 | Protease | Y222 | GWVLGPNNyYSFASQQQKPEDTTIPSTELAK | SEQ ID |
| | | | (proteasomal | | | NO: 140 |
| | | | subunit) | | | |
| 141 | PSMD8 | NP_002803.1 | Protease | Y223 | GWVLGPNNyYSFASQQQKPEDTTIPSTELAK | SEQ ID |
| | | | (proteasomal | | | NO: 141 |
| | | | subunit) | | | |
| 142 | ADRA2B | NP_000673.2 | Receptor, GPCR | Y120 | ALEyNSKRTPR | SEQ ID |
| | | | | | | NO: 142 |
| 143 | CELSR1 | NP_055061.1 | Receptor, GPCR | Y390 | DSPINANLRyR | SEQ ID |
| | | | | | | NO: 143 |
| 144 | OR5B3 | NP_001005489.1 | Receptor, GPCR | Y288 | PMLSPIVyTLRNKDV | SEQ ID |
| | | | | | | NO: 144 |
| 145 | MLNR | NP_001498.1 | Receptor, misc. | Y96 | DMRTTTNLYLGSMAVSDLLILLGLPFDLyR | SEQ ID |
| | | | | | | NO: 145 |
| 146 | PTDSR | NP_055982.1 | Receptor, misc. | Y116 | SVKMKMKyYIEYMES | SEQ ID |
| | | | | | | NO: 146 |
| 147 | PTDSR | NP_055982.1 | Receptor, misc. | Y137 | LYIFDSSyGEHPKRR | SEQ ID |
| | | | | | | NO: 147 |
| 148 | PTDSR | NP_055982.1 | Receptor, misc. | Y67 | YERPyKPVVLLNAQEGWSAQEK | SEQ ID |
| | | | | | | NO: 148 |
| 149 | SLAMF6 | | Receptor, misc. | Y295 | GSPGNTVyAQVTRPM | SEQ ID |
| | | | | | | NO: 149 |
| 150 | SLAMF6 | | Receptor, misc. | Y319 | KNDSMTIySIVNHSR | SEQ ID |
| | | | | | | NO: 150 |
| 151 | FXR1 | NP_001013456.1 | RNA binding | Y68 | EISEGDEVEVySR | SEQ ID |
| | | | protein | | | NO: 151 |
| 152 | HNRPL | NP_001005335.1 | RNA binding | Y441 | NPNGPyPYTLK | SEQ ID |
| | | | protein | | | NO: 152 |
| 153 | HNRPL | NP_001005335.1 | RNA binding | Y443 | NPNGPYPyTLK | SEQ ID |
| | | | protein | | | NO: 153 |
| 154 | IMP3 | NP_060755.1 | RNA binding | Y84 | ASAALLDKLyALGLVPTR | SEQ ID |
| | | | protein | | | NO: 154 |
| 155 | MVP | NP_005106.2 | RNA binding | Y15 | IPPYHyIHVLDQNSNVSR | SEQ ID |
| | | | protein | | | NO: 155 |
| 156 | NUP160 | NP_056046.1 | RNA binding | Y355 | YSPTMGLyLGIYMHA | SEQ ID |
| | | | protein | | | NO: 156 |
| 157 | PABPC1 | NP_002559.2 | RNA binding | Y56 | RSLGYAyVNFQQPADAER | SEQ ID |
| | | | protein | | | NO: 157 |
| 158 | PABPN1 | NP_004634.1 | RNA binding | Y217 | GFAyIEFSDKESVR | SEQ ID |
| | | | protein | | | NO: 158 |
| 159 | PUM2 | NP_056132.1 | RNA binding | Y1045 | yYLKNSPDLGPIGGPPNGML | SEQ ID |
| | | | protein | | | NO: 159 |
| 160 | RALY | NP_031393.2 | RNA binding | Y57 | VAGCSVHKGyAFVQYSNER | SEQ ID |
| | | | protein | | | NO: 160 |
| 161 | RNPS1 | NP_006702.1 | RNA binding | Y207 | MHPHLSKGYAyVEFENPDEAEK | SEQ ID |
| | | | protein | | | NO: 161 |
| 162 | TIA1 | NP_071320.1 | RNA binding | Y48 | MIMDTAGNDPyCFVEFHEHR | SEQ ID |
| | | | protein | | | NO: 162 |
| 163 | GKN1 | NP_062563.3 | Secreted protein | Y119 | PPPKGLMySVNPNKV | SEQ ID |
| | | | | | | NO: 163 |
| 164 | HDGF | NP_004485.1 | Secreted protein | Y45 | STANKyQVFFFGTHETAFLGPK | SEQ ID |
| | | | | | | NO: 164 |
| 165 | NTS | NP_006174.1 | Secreted protein | Y146 | IPyILKRQLYENKPRR | SEQ ID |
| | | | | | | NO: 165 |
| 166 | TGFB1 | NP_000651.3 | Secreted protein | Y284 | RRALDTNyCFSSTEK | SEQ ID |
| | | | | | | NO: 166 |
| 167 | CTCF | NP_006556.1 | Transcription | Y407 | THSGEKPyECYICHAR | SEQ ID |
| | | | factor | | | NO: 167 |
| 168 | GATA6 | NP_005248.2 | Transcription | Y417 | DGTGHYLCNACGLySK | SEQ ID |
| | | | factor | | | NO: 168 |
| 169 | HOXC8 | NP_073149.1 | Transcription | Y23 | AGESLEPAyYDCR | SEQ ID |
| | | | factor | | | NO: 169 |
| 170 | MLL | NP_005924.2 | Transcription | Y3914 | FINHSCEPNCySR | SEQ ID |
| | | | factor | | | NO: 170 |
| 171 | NFKB2 | NP_002493.2 | Transcription | Y285 | FYEDDENGWQAFGDFSPTDVHKQyAIVFR | SEQ ID |
| | | | factor | | | NO: 171 |
| 172 | STAT5B | NP_036580.2 | Transcription | Y665 | LGDLNyLIYVFPDRPK | SEQ ID |
| | | | factor | | | NO: 172 |
| 173 | TBP | NP_003185.1 | Transcription | Y322 | AEIyEAFENIYPILK | SEQ ID |
| | | | factor | | | NO: 173 |
| 174 | ZNF143 | NP_003433.2 | Transcription | Y345 | THTGERPyYCTEPGCGR | SEQ ID |
| | | | factor | | | NO: 174 |
| 175 | ZNF324 | NP_009057.1 | Transcription | Y313 | IHSGETPyACPVCGK | SEQ ID |
| | | | factor | | | NO: 175 |
| 176 | ZNF616 | NP_848618.2 | Transcription | Y297 | IHTGEKPyKCNLCGK | SEQ ID |
| | | | factor | | | NO: 176 |
| 177 | ZNFN1A3 | NP_036613.2 | Transcription | Y96 | EYNEyENIKLER | SEQ ID |
| | | | factor | | | NO. 177 |
| 178 | CNOT1 | NP_057368.3 | Transcription | Y851 | EIDDEANSyFQR | SEQ ID |
| | | | initiation | | | NO: 178 |
| | | | complex | | | |
| 179 | GTF3C5 | NP_036219.1 | Transcription | Y316 | IyQVLDFR | SEQ ID |
| | | | initiation | | | NO: 179 |
| | | | complex | | | |
| 180 | POLR2A | NP_000928.1 | Transcription | Y1881 | YSPTSPTySPTTPK | SEQ ID |
| | | | initiation | | | NO: 180 |
| | | | complex | | | |
| 181 | SUI1 | NP_005792.1 | Transcription | Y79 | FACNGTVIEHPEyGEVIQLQGDQR | SEQ ID |
| | | | initiation | | | NO: 181 |
| | | | complex | | | |
| 182 | SUPT16H | NP_009123.1 | Transcription | Y565 | NISMSVEGDyTYLR | SEQ ID |
| | | | initiation | | | NO: 182 |
| | | | complex | | | |
| 183 | C19orf2 | NP_003787.2 | Transcription, | Y392 | NSTGSGHSAQELPTIRTPADIyR | SEQ ID |
| | | | coactivator/ | | | NO: 183 |
| | | | corepressor | | | |
| 184 | CRSP2 | NP_004220.2 | Transcription, | Y901 | TNTAyQCFSILPQSSTHIR | SEQ ID |
| | | | coactivator/ | | | NO: 184 |
| | | | corepressor | | | |
| 185 | NMI | NP_004679.1 | Transcription, | Y238 | VTVSPyTEIHLK | SEQ ID |
| | | | coactivator/ | | | NO: 185 |
| | | | corepressor | | | |
| 186 | PHB2 | NP_009204.1 | Transcription, | Y77 | IPWFQyPIIYDIR | SEQ ID |
| | | | coactivator/ | | | NO: 186 |
| | | | corepressor | | | |
| 187 | SAP130 | NP_078821.2 | Transcription, | Y966 | VHLCAAQLLQLTNLEHDVyER | SEQ ID |
| | | | coactivator/ | | | NO: 187 |
| | | | corepressor | | | |
| 188 | TP53BP2 | NP_005417.1 | Transcription, | Y487 | KPQTVAASSIySMYTQQQAPGK | SEQ ID |
| | | | coactivator/ | | | NO: 188 |
| | | | corepressor | | | |
| 189 | ZHX2 | NP_055758.1 | Transcription, | Y470 | ASFLQSQFPDDAEVyRLIEVTGLAR | SEQ ID |
| | | | coactivator/ | | | NO: 189 |
| | | | corepressor | | | |
| 190 | ATIC | NP_004035.2 | Transferase | Y104 | VVACNLyPFVK | SEQ ID |
| | | | | | | NO: 190 |
| 191 | ATIC | NP_004035.2 | Transferase | Y192 | AFTHTAQYDEAISDyFR | SEQ ID |
| | | | | | | NO: 191 |
| 192 | ATIC | | Transferase | Y197 | SDYFRKQySKGISQM | SEQ ID |
| | | | | | | NO: 192 |
| 193 | ATIC | NP_004035.2 | Transferase | Y208 | yGMNPHQTPAQLYTLQPK | SEQ ID |
| | | | | | | NO: 193 |
| 194 | ATIC | NP_004035.2 | Transferase | Y220 | YGMNPHQTPAQLyTLQPK | SEQ ID |
| | | | | | | NO: 194 |
| 195 | GALNT12 | NP_078918.2 | Transferase | Y132 | EKKYDyDNLPR | SEQ ID |
| | | | | | | NO: 195 |
| 196 | GALNT9 | NP_068580.2 | Transferase | Y19 | PyNNDIDYYAK | SEQ ID |
| | | | | | | NO: 196 |
| 197 | GALNT9 | NP_068580.2 | Transferase | Y25 | PYNNDIDyYAK | SEQ ID |
| | | | | | | NO: 197 |
| 198 | GNPNAT1 | NP_932332.1 | Transferase | Y177 | FGYTVSEENyMCR | SEQ ID |
| | | | | | | NO: 198 |
| 199 | HS6ST1 | NP_004798.2 | Transferase | Y169 | FyYITLLR | SEQ ID |
| | | | | | | NO: 199 |
| 200 | NDST1 | NP_001534.1 | Transferase | Y427 | GIPTDMGyAVAPHHS | SEQ ID |
| | | | | | | NO: 200 |
| 201 | NDST1 | NP_001534.1 | Transferase | Y437 | APHHSGVyPVHVQLY | SEQ ID |
| | | | | | | NO: 201 |
| 202 | Sprn | NP_001012526.2 | Transferase | Y56 | RVRPAQRyGAPGSSL | SEQ ID |
| | | | | | | NO: 202 |
| 203 | EIF3S7 | NP_003744.1 | Translation | Y446 | WTCCALLAGSEyLK | SEQ ID |
| | | | initiation | | | NO: 203 |
| | | | complex | | | |
| 204 | RPL21 | NP_000973.2 | Translation | Y34 | IyKKGDIVDIKGMGTVQKGMPHKCYHGK | SEQ ID |
| | | | initiation | | | NO: 204 |
| | | | complex | | | |
| 205 | RPL21 | NP_000973.2 | Translation | Y57 | YHQHLQEQLDLDLSPLEYMMKSyPEIK | SEQ ID |
| | | | initiation | | | NO: 205 |
| | | | complex | | | |
| 206 | ABCF2 | NP_005683.2 | Transporter, ABC | Y492 | YHQHLQEQLDLDLSPLEYMMKCyPEIK | SEQ ID |
| | | | | | | NO: 206 |
| 207 | PITPNA | NP_006215.1 | Transporter, | Y93 | AWNAYPyCR | SEQ ID |
| | | | facilitator | | | NO: 207 |
| 208 | SLC13A3 | NP_001011554.1 | Transporter, | Y193 | GFLISIPySASIGGTATLTGTAPNLILLGQLK | SEQ ID |
| | | | facilitator | | | NO: 208 |
| 209 | SLC25A13 | NP_055066.1 | Transporter, | Y371 | TRMQNQRSTGSFVGELMyKNSFDCFK | SEQ ID |
| | | | facilitator | | | NO: 209 |
| 210 | APC | NP_000029.2 | Tumor suppressor | Y2645 | TLIyQMAPAVSK | SEQ ID |
| | | | | | | NO: 210 |
| 211 | ANAPC2 | NP_037498.1 | Ubiquitin | Y810 | DQQLVySAGVYR | SEQ ID |
| | | | conjugating | | | NO: 211 |
| | | | system | | | |
| 212 | DTX3L | NP_612144.1 | Ubiquitin | Y235 | SNyFEVPLPYFEYFK | SEQ ID |
| | | | conjugating | | | NO: 212 |
| | | | system | | | |
| 213 | DTX3L | NP_612144.1 | Ubiquitin | Y719 | FGGPEMyGYPDPSYLKR | SEQ ID |
| | | | conjugating | | | NO: 213 |
| | | | system | | | |
| 214 | USP11 | NP_004642.2 | Ubiquitin | Y870 | DLDFSEFVIQPQNESNPELyK | SEQ ID |
| | | | conjugating | | | NO: 214 |
| | | | system | | | |
| 215 | USP25 | NP_037528.3 | Ubiquitin | Y69 | TPQQEETTyYQTALPGNDR | SEQ ID |
| | | | conjugating | | | NO: 215 |
| | | | system | | | |
| 216 | AP2B1 | | Vesicle protein | Y737 | ISGTFTHRQGHIyME | SEQ ID |
| | | | | | | NO: 216 |
| 217 | CORO7 | NP_078811.2 | Vesicle protein | Y615 | FHPLAANVLASSSyDLTVR | SEQ ID |
| | | | | | | NO: 217 |
| 218 | SBLF | NP_006864.2 | Vesicle protein | Y628 | YESAyQAVVWK | SEQ ID |
| | | | | | | NO: 218 |
| 219 | VPS35 | NP_060676.2 | Vesicle protein | Y791 | ESPESEGPIyEGLIL | SEQ ID |
| | | | | | | NO: 219 |

The short name for each protein in which a phosphorylation site has presently been identified is provided in Column A, and it accession number (human) is provided Column B. The protein type/group into which each protein falls is provided in Column C. The identified tyrosine residue at which phosphorylation occurs in a given protein is identified in Column D, and the amino acid sequence of the phosphorylation site encompassing the tyrosine residue is provided in Column E (lower case y=the tyrosine (identified in Column D) at which phosphorylation occurs. Table 1 above is identical to FIG. 2, except that the latter includes the disease and cell type(s) in which the particular phosphorylation site was identified (Columns F and G).

The identification of these 219 phosphorylation sites is described in more detail in Part A below and in Example 1.

### DEFINITIONS

As used herein, the following terms have the meanings indicated:

“Antibody” or “antibodies” refers to all types of immunoglobulins, including IgG, IgM, IgA, IgD, and IgE, including F_{ab} or antigen-recognition fragments thereof, including chimeric, polyclonal, and monoclonal antibodies. The term “does not bind” with respect to an antibody's binding to one phospho-form of a sequence means does not substantially react with as compared to the antibody's binding to the other phospho-form of the sequence for which the antibody is specific.

“ALCL-related signaling protein” means any protein (or polypeptide derived therefrom) enumerated in Column A of Table 1/FIG. 2, which is disclosed herein as being phosphorylated in one or more Anaplastic Large Cell Lymphoma (ALCL) cell line(s). An ALCL-related signaling protein may also be phosphorylated in other non-ALCL cell lines.

“Heavy-isotope labeled peptide” (used interchangeably with AQUA peptide) means a peptide comprising at least one heavy-isotope label, which is suitable for absolute quantification or detection of a protein as described in WO/03016861, “Absolute Quantification of Proteins and Modified Forms Thereof by Multistage Mass Spectrometry” (Gygi et al.), further discussed below.

“Protein” is used interchangeably with polypeptide, and includes protein fragments and domains as well as whole protein.

“Phosphorylatable amino acid” means any amino acid that is capable of being modified by addition of a phosphate group, and includes both forms of such amino acid.

“Phosphorylatable peptide sequence” means a peptide sequence comprising a phosphorylatable amino acid.

“Phosphorylation site-specific antibody” means an antibody that specifically binds a phosphorylatable peptide sequence/epitope only when phosphorylated, or only when not phosphorylated, respectively. The term is used interchangeably with “phospho-specific” antibody.

### A. Identification of Novel ALCL-Related Phosphorylation Sites.

The nearly 219 novel ALCL-related signaling protein phosphorylation sites disclosed herein and listed in Table 1/FIG. 2 were discovered by employing the modified peptide isolation and characterization techniques described in described in “Immunoaffinity Isolation of Modified Peptides From Complex Mixtures,” U.S. Patent Publication No. 20030044848, Rush et al. (the teaching of which is hereby incorporated herein by reference, in its entirety) using cellular extracts from recognized ALCL tumor cell lines as indicated in Column G. Exemplary cells used in the peptide isolation methods described herein and in Rush et al. include H526, MOLT15, SUP-M2, 293T ATIC-ALK TTS, 293T NPM-ALK TTS, TS, SR-786, and Karpas 299. The isolation and identification of phosphopeptides from these ALCL cell lines, using an immobilized general phosphotyrosine-specific antibody, is described in detail in Example 1 below. In addition to the nearly 219 previously unknown protein phosphorylation sites discovered, many known phosphorylation sites were also identified (but are described herein). The immunoaffinity/mass spectrometric technique described in the '848 Patent Publication (the “IAP” method)—and employed as described in detail in the Examples—is briefly summarized below.

The IAP method employed generally comprises the following steps: (a) a proteinaceous preparation (e.g. a digested cell extract) comprising phosphopeptides from two or more different proteins is obtained from an organism; (b) the preparation is contacted with at least one immobilized general phosphotyrosine-specific antibody; (c) at least one phosphopeptide specifically bound by the immobilized antibody in step (b) is isolated; and (d) the modified peptide isolated in step (c) is characterized by mass spectrometry (MS) and/or tandem mass spectrometry (MS-MS). Subsequently, (e) a search program (e.g. Sequest) may be utilized to substantially match the spectra obtained for the isolated, modified peptide during the characterization of step (d) with the spectra for a known peptide sequence. A quantification step employing, e.g. SILAC or AQUA, may also be employed to quantify isolated peptides in order to compare peptide levels in a sample to a baseline.

In the IAP method as employed herein, a general phosphotyrosine-specific monoclonal antibody (commercially available from Cell Signaling Technology, Inc., Beverly, Mass., Cat #9411 (p-Tyr-100)) was used in the immunoaffinity step to isolate the widest possible number of phospho-tyrosine containing peptides from the ALCL cell extracts.

As described in more detail in the Examples, lysates were prepared from both cell lines and digested with trypsin after treatment with DTT and iodoacetamide to alkylate cysteine residues. Before the immunoaffinity step, peptides were prefractionated by reversed-phase solid phase extraction using Sep-Pak C₁₈ columns to separate peptides from other cellular components. The solid phase extraction cartridges were eluted with varying steps of acetonitrile. Each lyophilized peptide fraction was redissolved in MOPS buffer and treated with phosphotyrosine antibody (P-Tyr-100, CST #9411) immobilized on protein G-Sepharose. Immunoaffinity-purified peptides were eluted with 0.1% TFA and a portion of this fraction was concentrated with Zip-Tips and analyzed by LC-MS/MS, using a ThermoFinnigan LCQ Deca XP Plus ion trap mass spectrometer. Peptides were eluted from a 10 cm×75 μm reversed-phase column with a 45-min linear gradient of acetonitrile. MS/MS spectra were evaluated using the program Sequest with the NCBI human protein database.

As a result of the discovery of these phosphorylation sites, phospho-specific antibodies and AQUA peptides for the detection of and quantification of these sites and their parent proteins may now be produced by standard methods, described below. These new reagents will prove highly useful in studying the signaling pathways and events underlying the progression of ALCL and the identification of new biomarkers and targets for its diagnosis and treatment.

### B. Antibodies and Cell Lines

Isolated phosphorylation site specific antibodies that specifically bind an ALCL-related signaling protein disclosed in Column A of Table 1 only when phosphorylated (or only when not phosphorylated) at the corresponding amino acid and phosphorylation site listed in Column D of Table 1 may now be produced by standard antibody production methods, such as anti-peptide antibody methods, using the phosphorylation site sequence information provided in Column E of Table 1. For example, a new MAPK6 phosphorylation sites (tyrosine 628) (see Row 100 of Table 1) are presently disclosed. Thus, antibodies that specifically bind this novel MAPK6 site can now be produced by using (all or part of) the amino acid sequence encompassing the respective phosphorylated residue as a peptide antigen used to immunize an animal (e.g. a peptide antigen comprising the sequence set forth in Row 100, Column E, of Table 1 (which encompasses the phosphorylated tyrosine as position 628 in MAPK6) may be employed to produce an antibody that only binds MAPK6 when phosphorylated at tyr628).

Polyclonal antibodies of the invention may be produced according to standard techniques by immunizing a suitable animal (e.g., rabbit, goat, etc.) with a peptide antigen corresponding to the ALCL-related phosphorylation site of interest (i.e. a phosphorylation site enumerated in Column E of Table 1, which comprises the corresponding phosphorylatable amino acid listed in Column E of Table 1), collecting immune serum from the animal, and separating the polyclonal antibodies from the immune serum, in accordance with known procedures. For example, a peptide antigen comprising the novel GAB3 phosphorylation site disclosed herein (SEQ ID NO: 9=SPSAEDSyVPMSPKG, encompassing phosphorylated tyrosine 395 (see Row 9 of Table 1)) may be used to produce antibodies that only bind GAB3 when phosphorylated at Tyr395. Similarly, a peptide comprising any of the phosphorylation site sequences provided in Column E of Table 1 may employed as an antigen to produce an antibody that only binds the corresponding protein listed in Column A of Table 1 when phosphorylated (or when not phosphorylated) at the corresponding residue listed in Column D. If an antibody that only binds the protein when phosphorylated at the disclosed site is desired, the peptide antigen includes the phosphorylated form of the amino acid. Conversely, if an antibody that only binds the protein when not phosphorylated at the disclosed site is desired, the peptide antigen includes the non-phosphorylated form of the amino acid.

Peptide antigens suitable for producing antibodies of the invention may be designed, constructed and employed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85: 21-49 (1962)).

It will be appreciated by those of skill in the art that longer or shorter phosphopeptide antigens may be employed. See Id. For example, a peptide antigen may consist of the full sequence disclosed in Column E of Table 1, or it may comprise additional amino acids flanking such disclosed sequence, or may comprise of only a portion of the disclosed sequence immediately flanking the phosphorylatable amino acid (indicated in Column E by lowercase “y”). Polyclonal antibodies produced as described herein may be screened as further described below.

Monoclonal antibodies of the invention may be produced in a hybridoma cell line according to the well-known technique of Kohler and Milstein. Nature 265: 495-97 (1975); Kohler and Milstein, Eur. J. Immunol. 6: 511 (1976); see also, CURRENT PROTOCOLS IN MOLECULAR BIOLOGY, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse or other species and, after a sufficient time (in keeping with conventional techniques), the animal is sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063, C. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246: 1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)).

The preferred epitope of a phosphorylation-site specific antibody of the invention is a peptide fragment consisting essentially of about 8 to 17 amino acids including the phosphorylatable tyrosine, wherein about 3 to 8 amino acids are positioned on each side of the phosphorylatable tyrosine (for example, the CBLB tyrosine 276 phosphorylation site sequence disclosed in Row 23, Column E of Table 1), and antibodies of the invention thus specifically bind a target ALCL polypeptide comprising such epitopic sequence. Particularly preferred epitopes bound by the antibodies of the invention comprise all or part of a phosphorylatable site sequence listed in Column E of Table 1, including the phosphorylatable amino acid.

Included in the scope of the invention are equivalent non-antibody molecules, such as protein binding domains or nucleic acid aptamers, which bind, in a phospho-specific manner, to essentially the same phosphorylatable epitope to which the phospho-specific antibodies of the invention bind. See, e.g., Neuberger et al., Nature 312: 604 (1984). Such equivalent non-antibody reagents may be suitably employed in the methods of the invention further described below.

Antibodies provided by the invention may be any type of immunoglobulins, including IgG, IgM, IgA, IgD, and IgE, including F_{ab} or antigen-recognition fragments thereof. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed by specific antibodies made according to the method disclosed in U.S. Pat. No. 4,676,980 (Segel et al.)

The invention also provides immortalized cell lines that produce an antibody of the invention. For example, hybridoma clones, constructed as described above, that produce monoclonal antibodies to the ALCL-related signaling protein phosphorylation sties disclosed herein are also provided. Similarly, the invention includes recombinant cells producing an antibody of the invention, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

Phosphorylation site-specific antibodies of the invention, whether polyclonal or monoclonal, may be screened for epitope and phospho-specificity according to standard techniques. See, e.g. Czemik et al., Methods in Enzymology, 201: 264-283 (1991). For example, the antibodies may be screened against the phospho and non-phospho peptide library by ELISA to ensure specificity for both the desired antigen (i.e. that epitope including a phosphorylation site sequence enumerated in Column E of Table 1) and for reactivity only with the phosphorylated (or non-phosphorylated) form of the antigen. Peptide competition assays may be carried out to confirm lack of reactivity with other phospho-epitopes on the given ALCL-related signaling protein. The antibodies may also be tested by Western blotting against cell preparations containing the signaling protein, e.g. cell lines over-expressing the target protein, to confirm reactivity with the desired phosphorylated epitope/target.

Specificity against the desired phosphorylated epitope may also be examined by constructing mutants lacking phosphorylatable residues at positions outside the desired epitope known to be phosphorylated, or by mutating the desired phospho-epitope and confirming lack of reactivity. Phosphorylation-site specific antibodies of the invention may exhibit some limited cross-reactivity related epitopes in non-target proteins. This is not unexpected as most antibodies exhibit some degree of cross-reactivity, and anti-peptide antibodies will often cross-react with epitopes having high homology to the immunizing peptide. See, e.g., Czemik, supra. Cross-reactivity with non-target proteins is readily characterized by Western blotting alongside markers of known molecular weight. Amino acid sequences of cross-reacting proteins may be examined to identify sites highly homologous to the ALCL-related signaling protein epitope for which the antibody of the invention is specific. In certain cases, polyclonal antisera may be exhibit some undesirable general cross-reactivity to phosphotyrosine, which may be removed by further purification of antisera, e.g. over a phosphotyramine column. Antibodies of the invention specifically bind their target protein (i.e. a protein listed in Column A of Table 1) only when phosphorylated (or only when not phosphorylated, as the case may be) at the site disclosed in corresponding Column D, and do not (substantially) bind to the other form (as compared to the form for which the antibody is specific).

Antibodies may be further characterized via immunohistochemical (IHC) staining using normal and diseased tissues to examine ALCL-related phosphorylation and activation status in diseased tissue. IHC may be carried out according to well known techniques. See, e.g., ANTIBODIES: A LABORATORY MANUAL, Chapter 10, Harlow & Lane Eds., Cold Spring Harbor Laboratory (1988). Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies may be further characterized by flow cytometry carried out according to standard methods. See Chow et al., Cytometry (Communications in Clinical Cytometry) 46: 72-78 (2001). Briefly and by way of example, the following protocol for cytometric analysis may be employed: samples may be centrifuged on Ficoll gradients to remove erythrocytes, and cells may then be fixed with 2% paraformaldehyde for 10 minutes at 37° C. followed by permeabilization in 90% methanol for 30 minutes on ice. Cells may then be stained with the primary phosphorylation-site specific antibody of the invention (which detects an ALCL-related signal transduction protein enumerated in Table 1), washed and labeled with a fluorescent-labeled secondary antibody. Additional fluorochrome-conjugated marker antibodies (e.g. CD45, CD34) may also be added at this time to aid in the subsequent identification of specific hematopoietic cell types. The cells would then be analyzed on a flow cytometer (e.g. a Beckman Coulter FC500) according to the specific protocols of the instrument used.

Antibodies of the invention may also be advantageously conjugated to fluorescent dyes (e.g. Alexa488, PE) for use in multi-parametric analyses along with other signal transduction (phospho-CrkL, phospho-Erk 1/2) and/or cell marker (CD34) antibodies.

Phosphorylation-site specific antibodies of the invention specifically bind to a human ALCL-related signal transduction protein only when phosphorylated at a disclosed site, but are not limited only to binding the human species, per se. The invention includes antibodies that also bind conserved and highly-homologous phosphorylation sites in respective ALCL-related proteins from other species (e.g. mouse, rat, monkey, yeast), in addition to binding the human phosphorylation site. Highly-homologous sites conserved in other species can readily be identified by standard sequence comparisons, such as using BLAST, with the human ALCL-signal transduction protein phosphorylation sites disclosed herein.

### C. Heavy-Isotope Labeled Peptides (AQUA Peptides).

The novel ALCL-signaling protein phosphorylation sites disclosed herein now enable the production of corresponding heavy-isotope labeled peptides for the absolute quantification of such signaling proteins (both phosphorylated and not phosphorylated at a disclosed site) in biological samples. The production and use of AQUA peptides for the absolute quantification of proteins (AQUA) in complex mixtures has been described. See WO/03016861, “Absolute Quantification of Proteins and Modified Forms Thereof by Multistage Mass Spectrometry,” Gygi et al. and also Gerber et al. Proc. Natl. Acad. Sci. U.S.A. 100: 6940-5 (2003) (the teachings of which are hereby incorporated herein by reference, in their entirety).

The AQUA methodology employs the introduction of a known quantity of at least one heavy-isotope labeled peptide standard (which has a unique signature detectable by LC-SRM chromatography) into a digested biological sample in order to determine, by comparison to the peptide standard, the absolute quantity of a peptide with the same sequence and protein modification in the biological sample. Briefly, the AQUA methodology has two stages: peptide internal standard selection and validation and method development; and implementation using validated peptide internal standards to detect and quantify a target protein in sample. The method is a powerful technique for detecting and quantifying a given peptide/protein within a complex biological mixture, such as a cell lysate, and may be employed, e.g., to quantify change in protein phosphorylation as a result of drug treatment, or to quantify differences in the level of a protein in different biological states.

Generally, to develop a suitable internal standard, a particular peptide (or modified peptide) within a target protein sequence is chosen based on its amino acid sequence and the particular protease to be used to digest. The peptide is then generated by solid-phase peptide synthesis such that one residue is replaced with that same residue containing stable isotopes (¹³C, ¹⁵N). The result is a peptide that is chemically identical to its native counterpart formed by proteolysis, but is easily distinguishable by MS via a mass shift. The newly synthesized AQUA internal standard peptide is then evaluated by LC-MS/MS. This process provides qualitative information about peptide retention by reverse-phase chromatography, ionization efficiency, and fragmentation via collision-induced dissociation. Informative and abundant fragment ions for sets of native and internal standard peptides are chosen and then specifically monitored in rapid succession as a function of chromatographic retention to form a selected reaction monitoring (LC-SRM) method based on the unique profile of the peptide standard.

The second stage of the AQUA strategy is its implementation to measure the amount of a protein or modified protein from complex mixtures. Whole cell lysates are typically fractionated by SDS-PAGE gel electrophoresis, and regions of the gel consistent with protein migration are excised. This process is followed by in-gel proteolysis in the presence of the AQUA peptides and LC-SRM analysis. (See Gerber et al. supra.) AQUA peptides are spiked in to the complex peptide mixture obtained by digestion of the whole cell lysate with a proteolytic enzyme and subjected to immunoaffinity purification as described above. The retention time and fragmentation pattern of the native peptide formed by digestion (e.g. trypsinization) is identical to that of the AQUA internal standard peptide determined previously; thus, LC-MS/MS analysis using an SRM experiment results in the highly specific and sensitive measurement of both internal standard and analyte directly from extremely complex peptide mixtures. Because an absolute amount of the AQUA peptide is added (e.g. 250 fmol), the ratio of the areas under the curve can be used to determine the precise expression levels of a protein or phosphorylated form of a protein in the original cell lysate. In addition, the internal standard is present during in-gel digestion as native peptides are formed, such that peptide extraction efficiency from gel pieces, absolute losses during sample handling (including vacuum centrifugation), and variability during introduction into the LC-MS system do not affect the determined ratio of native and AQUA peptide abundances.

An AQUA peptide standard is developed for a known phosphorylation site sequence previously identified by the IAP-LC-MS/MS method within in a target protein. One AQUA peptide incorporating the phosphorylated form of the particular residue within the site may be developed, and a second AQUA peptide incorporating the non-phosphorylated form of the residue developed. In this way, the two standards may be used to detect and quantify both the phosphorylated and non-phosphorylated forms of the site in a biological sample.

Peptide internal standards may also be generated by examining the primary amino acid sequence of a protein and determining the boundaries of peptides produced by protease cleavage. Alternatively, a protein may actually be digested with a protease and a particular peptide fragment produced can then sequenced. Suitable proteases include, but are not limited to, serine proteases (e.g. trypsin, hepsin), metallo proteases (e.g. PUMP1), chymotrypsin, cathepsin, pepsin, thermolysin, carboxypeptidases, etc.

A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Thus, a peptide is preferably at least about 6 amino acids. The size of the peptide is also optimized to maximize ionization frequency. Thus, peptides longer than about 20 amino acids are not preferred. The preferred ranged is about 7 to 15 amino acids. A peptide sequence is also selected that is not likely to be chemically reactive during mass spectrometry, thus sequences comprising cysteine, tryptophan, or methionine are avoided.

A peptide sequence that does not include a modified region of the target region may be selected so that the peptide internal standard can be used to determine the quantity of all forms of the protein. Alternatively, a peptide internal standard encompassing a modified amino acid may be desirable to detect and quantify only the modified form of the target protein. Peptide standards for both modified and unmodified regions can be used together, to determine the extent of a modification in a particular sample (i.e. to determine what fraction of the total amount of protein is represented by the modified form). For example, peptide standards for both the phosphorylated and unphosphorylated form of a protein known to be phosphorylated at a particular site can be used to quantify the amount of phosphorylated form in a sample.

The peptide is labeled using one or more labeled amino acids (i.e. the label is an actual part of the peptide) or less preferably, labels may be attached after synthesis according to standard methods. Preferably, the label is a mass-altering label selected based on the following considerations: The mass should be unique to shift fragments masses produced by MS analysis to regions of the spectrum with low background; the ion mass signature component is the portion of the labeling moiety that preferably exhibits a unique ion mass signature in MS analysis; the sum of the masses of the constituent atoms of the label is preferably uniquely different than the fragments of all the possible amino acids. As a result, the labeled amino acids and peptides are readily distinguished from unlabeled ones by the ion/mass pattern in the resulting mass spectrum. Preferably, the ion mass signature component imparts a mass to a protein fragment that does not match the residue mass for any of the 20 natural amino acids.

The label should be robust under the fragmentation conditions of MS and not undergo unfavorable fragmentation. Labeling chemistry should be efficient under a range of conditions, particularly denaturing conditions, and the labeled tag preferably remains soluble in the MS buffer system of choice. The label preferably does not suppress the ionization efficiency of the protein and is not chemically reactive. The label may contain a mixture of two or more isotopically distinct species to generate a unique mass spectrometric pattern at each labeled fragment position. Stable isotopes, such as ¹³C, ¹⁵N, ¹⁷O, ¹⁸O, or ³⁴S, are among preferred labels. Pairs of peptide internal standards that incorporate a different isotope label may also be prepared. Preferred amino acid residues into which a heavy isotope label may be incorporated include leucine, proline, valine, and phenylalanine.

Peptide internal standards are characterized according to their mass-to-charge (m/z) ratio, and preferably, also according to their retention time on a chromatographic column (e.g. an HPLC column). Internal standards that co-elute with unlabeled peptides of identical sequence are selected as optimal internal standards. The internal standard is then analyzed by fragmenting the peptide by any suitable means, for example by collision-induced dissociation (CID) using, e.g., argon or helium as a collision gas. The fragments are then analyzed, for example by multi-stage mass spectrometry (MSⁿ) to obtain a fragment ion spectrum, to obtain a peptide fragmentation signature. Preferably, peptide fragments have significant differences in m/z ratios to enable peaks corresponding to each fragment to be well separated, and a signature is that is unique for the target peptide is obtained. If a suitable fragment signature is not obtained at the first stage, additional stages of MS are performed until a unique signature is obtained.

Fragment ions in the MS/MS and MS³ spectra are typically highly specific for the peptide of interest, and, in conjunction with LC methods, allow a highly selective means of detecting and quantifying a target peptide/protein in a complex protein mixture, such as a cell lysate, containing many thousands or tens of thousands of proteins. Any biological sample potentially containing a target protein/peptide of interest may be assayed. Crude or partially purified cell extracts are preferably employed. Generally, the sample has at least 0.01 mg of protein, typically a concentration of 0.1-10 mg/mL, and may be adjusted to a desired buffer concentration and pH.

A known amount of a labeled peptide internal standard, preferably about 10 femtomoles, corresponding to a target protein to be detected/quantified is then added to a biological sample, such as a cell lysate. The spiked sample is then digested with one or more protease(s) for a suitable time period to allow digestion. A separation is then performed (e.g. by HPLC, reverse-phase HPLC, capillary electrophoresis, ion exchange chromatography, etc.) to isolate the labeled internal standard and its corresponding target peptide from other peptides in the sample. Microcapillary LC is a preferred method.

Each isolated peptide is then examined by monitoring of a selected reaction in the MS. This involves using the prior knowledge gained by the characterization of the peptide internal standard and then requiring the MS to continuously monitor a specific ion in the MS/MS or MSⁿ spectrum for both the peptide of interest and the internal standard. After elution, the area under the curve (AUC) for both peptide standard and target peptide peaks are calculated. The ratio of the two areas provides the absolute quantification that can be normalized for the number of cells used in the analysis and the protein's molecular weight, to provide the precise number of copies of the protein per cell. Further details of the AQUA methodology are described in Gygi et al., and Gerber et al. supra.

In accordance with the present invention, AQUA internal peptide standards (heavy-isotope labeled peptides) may now be produced, as described above, for any of the 219 novel ALCL-related signaling protein phosphorylation sites disclosed herein (see Table 1/FIG. 2). Peptide standards for a given phosphorylation site (e.g. the tyrosine 184 site in CAMK1—see Row 96 of Table 1) may be produced for both the phosphorylated and non-phosphorylated forms of the site (e.g. see CAMK1 site sequence in Column E, Row 96 of Table 1) and such standards employed in the AQUA methodology to detect and quantify both forms of such phosphorylation site in a biological sample.

The phosphorylation site peptide sequences disclosed herein (see Column E of Table 1/FIG. 2) are particularly well suited for development of corresponding AQUA peptides, since the IAP method by which they were identified (see Part A above and Example 1) inherently confirmed that such peptides are in fact produced by enzymatic digestion (trypsinization) and are in fact suitably fractionated/ionized in MS/MS. Thus, heavy-isotope labeled equivalents of these peptides (both in phosphorylated and unphosphorylated form) can be readily synthesized and their unique MS and LC-SRM signature determined, so that the peptides are validated as AQUA peptides and ready for use in quantification experiments.

Accordingly, the invention provides heavy-isotope labeled peptides (AQUA peptides) for the detection and/or quantification of any of the ALCL-related phosphorylation sites disclosed in Table 1 (see Column E) and/or their corresponding parent proteins (see Column A). Each such phosphorylation sequence may be considered a preferred AQUA peptide of the invention. Optimally, an AQUA peptide of the invention consists of a phosphorylation site sequence enumerated in Table 1. For example, an AQUA peptide comprising the sequence LETADAPARLEyYENAR (SEQ ID NO: 12) (where y may be either phosphotyrosine or tyrosine, and where L=labeled leucine (e.g. ¹⁴C)) is provided for the quantification of phosphorylated (or non-phosphorylated) IRS4 (tyr111) in a biological sample (see Row 12 of Table 1, tyrosine 111 being the phosphorylatable residue within the site). However, it will be appreciated that a larger AQUA peptide comprising the disclosed phosphorylation site sequence (and additional residues downstream or upstream of it) may also be constructed. Similarly, a smaller AQUA peptide comprising less than all of the residues of a disclosed phosphorylation site sequence (but still comprising the phosphorylatable residue enumerated in Column D) may alternatively be constructed. Such larger or shorter AQUA peptides are within the scope of the present invention, and the selection and production of preferred AQUA peptides may be carried out as described above (see Gygi et al., Gerber et al. supra.).

Certain particularly preferred subsets of AQUA peptides provided by the invention are described above (corresponding to particular protein types/groups in Table 1, for example, Kinase Proteins or Adaptor/Scaffold Proteins). Example 4 is provided to further illustrate the construction and use, by standard methods described above, of exemplary AQUA peptides provided by the invention. For example, AQUA peptides corresponding to the both the phosphorylated and non-phosphorylated forms of the disclosed IRS4 tyrosine 111 phosphorylation site (LETADAPARLEyYENAR (SEQ ID NO: 12)—see Row 12 of Table 1/FIG. 2) may be used to quantify the amount of phosphorylated IRS4 (tyr111) in biological sample, e.g. an ALCL tumor cell sample (or a sample before or after treatment with a test drug).

AQUA peptides of the invention may also be employed within a kit that comprises one or multiple AQUA peptide(s) provided herein (for the quantification of an ALCL-related signal transduction protein disclosed in Table 1), and, optionally, a second detecting reagent conjugated to a detectable group. For example, a kit may include AQUA peptides for both the phosphorylation and non-phosphorylated form of a phosphorylation site disclosed herein. The reagents may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

AQUA peptides provided by the invention will be highly useful in the further study of signal transduction anomalies underlying ALCL, and in identifying diagnostic/bio-markers of this disease, new potential drug targets, and/or in monitoring the effects of test compounds on ALCL-related signal transduction proteins and pathways.

### D. Immunoassay Formats

Antibodies provided by the invention may be advantageously employed in a variety of standard immunological assays (the use of AQUA peptides provided by the invention is described separately above). Assays may be homogeneous assays or heterogeneous assays. In a homogeneous assay the immunological reaction usually involves a phosphorylation-site specific antibody of the invention), a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In a heterogeneous assay approach, the reagents are usually the specimen, a phosphorylation-site specific antibody of the invention, and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof that may be useful for carrying out the methods disclosed herein are well known in the art. See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No. 4,659,678 (Forrest et al., “Immunoassay of Antigens”); U.S. Pat. No. 4,376,110 (David et al., “Immunometric Assays Using Monoclonal Antibodies”). Conditions suitable for the formation of reagent-antibody complexes are well described. See id. Monoclonal antibodies of the invention may be used in a “two-site” or “sandwich” assay, with a single cell line serving as a source for both the labeled monoclonal antibody and the bound monoclonal antibody. Such assays are described in U.S. Pat. No. 4,376,110. The concentration of detectable reagent should be sufficient such that the binding of a target ALCL-related signal transduction protein is detectable compared to background.

ALCL-related phosphorylation site-specific antibodies disclosed herein may be conjugated to a solid support suitable for a diagnostic assay (e.g., beads, plates, slides or wells formed from materials such as latex or polystyrene) in accordance with known techniques, such as precipitation. Antibodies, or other target protein or target site-binding reagents, may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

Antibodies of the invention may also be optimized for use in a flow cytometry assay to determine the activation/phosphorylation status of a target ALCL-related signal transduction protein in patients before, during, and after treatment with a drug targeted at inhibiting phosphorylation at such a protein at the phosphorylation site disclosed herein. For example, bone marrow cells or peripheral blood cells from patients may be analyzed by flow cytometry for target ALCL-related protein phosphorylation, as well as for markers identifying various hematopoietic cell types. In this manner, activation status of the malignant cells may be specifically characterized. Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 1% paraformaldehyde for 10 minutes at 37° C. followed by permeabilization in 90% methanol for 30 minutes on ice. Cells may then be stained with the primary antibody (a phospho-specific antibody of the invention), washed and labeled with a fluorescent-labeled secondary antibody. Alternatively, the cells may be stained with a fluorescent-labeled primary antibody. The cells would then be analyzed on a flow cytometer (e.g. a Beckman Coulter EPICS-XL) according to the specific protocols of the instrument used. Such an analysis would identify the presence of activated ALCL-related signal transduction protein(s)elated in the malignant cells and reveal the drug response on the targeted protein.

Alternatively, antibodies of the invention may be employed in immunohistochemical (IHC) staining to detect differences in signal transduction or protein activity using normal and diseased ALCL tissues. IHC may be carried out according to well-known techniques. See, e.g., ANTIBODIES: A LABORATORY MANUAL, supra. Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies of the invention may be also be optimized for use in other clinically-suitable applications, for example bead-based multiplex-type assays, such as IGEN, Luminex™ and/or Bioplex™ assay formats, or otherwise optimized for antibody arrays formats, such as reversed-phase array applications (see, e.g. Paweletz et al., Oncogene 20(16): 1981-89 (2001)). Accordingly, in another embodiment, the invention provides a method for the multiplex detection of ALCL-related protein phosphorylation in a biological sample, the method comprising utilizing at two or more antibodies or AQUA peptides of the invention to detect the presence of two or more phosphorylated ALCL-related signaling proteins enumerated in Column A of Table 1/FIG. 2. In one preferred embodiment, two to five antibodies or AQUA peptides of the invention are employed in the method. In another preferred embodiment, six to ten antibodies or AQUA peptides of the invention are employed, while in another preferred embodiment eleven to twenty are employed.

Antibodies and/or AQUA peptides of the invention may also be employed within a kit that comprises at least one phosphorylation site-specific antibody or AQUA peptide of the invention (which binds to or detects an ALCL-related signal transduction protein disclosed in Table 1), and, optionally, a second antibody conjugated to a detectable group. In some embodies, the kit is suitable for multiplex assays and comprises two or more antibodies or AQUA peptides of the invention, and in some embodiments, comprises two to five, six to ten, or eleven to twenty reagents of the invention. The kit may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

The following Examples are provided only to further illustrate the invention, and are not intended to limit its scope, except as provided in the claims appended hereto. The present invention encompasses modifications and variations of the methods taught herein which would be obvious to one of ordinary skill in the art.

### EXAMPLE 1

### Isolation of Phosphotyrosine-Containing Peptides from Extracts of Cells and Identification of Novel Phosphorylation Sites

In order to discover previously unknown ALCL-related signal transduction protein phosphorylation sites, IAP isolation techniques were employed to identify phosphotyrosine containing peptides in cell extracts, which are derived from anaplastic large cell lymphomas (ALCL). See Pulford et al. Blood 89: 394-1404 (1997). The majority of ALCL is characterized by the presence of the t(2,5)(p23;q35) chromosomal translocation that causes the fusion of the nucleophosmin and anaplastic lymphoma kinase genes. See Morris S W, Science 263: 1281-1284 (1994).

Tryptic phosphotyrosine peptides were purified and analyzed from extracts of the two ALCL cell lines as follows. Cells were grown in a 5% CO₂ incubator at 37° C. Cells were cultured to a density of 0.5-1.4×10⁶ cells/ml in RPMI 1640 medium containing 10% calf serum. Cells were washed with PBS at 4° C., resuspended at 1.25×10⁸ cells/ml in lysis buffer (20 mM HEPES pH 8.0, 9 M urea, 1 mM sodium vanadate) and sonicated. In some experiments, the PBS wash step was omitted.

Sonicated cell lysates were cleared by centrifugation at 20,000×g, and proteins were reduced with DTT at a final concentration of 4.1 mM and alkylated with iodoacetamide at 8.3 mM. For digestion with trypsin, protein extracts were diluted in 20 mM HEPES pH 8.0 to a final concentration of 2 M urea and immobilized TLCK-trypsin (Pierce) was added at 1-2.5 ml beads (200 TAME units trypsin/ml) per 10⁹ cells. For digestion with chymotrypsin, endoproteinase GIuC, and elastase, lysates were diluted in 20 mM HEPES pH 8.0 to a final concentration of 1 M urea, and GIuC (Worthington Biochemicals) or elastase (Roche) was added at 0.5 mg per 10⁹ cells. Chymotrypsin (Worthington Biochemicals) was added at 10 mg per 10⁹ cells. Digestion was performed for 1-2 days at room temperature.

Trifluoroacetic acid (TFA) was added to protein digests to a final concentration of 1%, precipitate was removed by centrifugation, and digests were loaded onto Sep-Pak C₁₈ columns (Waters) equilibrated with 0.1% TFA. A column volume of 0.7-1.0 ml was used per 2×10⁸ cells. Columns were washed with 15 volumes of 0.1% TFA, followed by 4 volumes of 5% acetonitrile (MeCN) in 0.1% TFA. Peptide fraction I was obtained by eluting columns with 2 volumes each of 8, 12, and 15% MeCN in 0.1% TFA and combining the eluates. Fractions II and III were a combination of eluates after eluting columns with 18, 22, 25% MeCN in 0.1% TFA and with 30, 35, 40% MeCN in 0.1% TFA, respectively. All peptide fractions were lyophilized.

Peptides from each fraction corresponding to 2×10⁸ cells were dissolved in 1 ml of IAP buffer (20 mM Tris/HCl or 50 mM MOPS pH 7.2, mM sodium phosphate, 50 mM NaCl) and insoluble matter (mainly in peptide fractions III) was removed by centrifugation. IAP was performed on each peptide fraction separately. The phosphotyrosine monoclonal antibody P-Tyr-100 (Cell Signaling Technology, Inc., catalog number 9411) was coupled at 4 mg/ml beads to protein G agarose (Roche). Immobilized antibody (15 μl, 60 μg) was added as 1:1 slurry in IAP buffer to 1 ml of each peptide fraction, and the mixture was incubated overnight at 4° C. with gentle rotation. The immobilized antibody beads were washed three times with 1 ml IAP buffer and twice with 1 ml water, all at 4° C. Peptides were eluted from beads by incubation with 75 μl of 0.1% TFA at room temperature for 10 min.

### Analysis by MALDI-TOF Mass Spectrometry.

A thin layer of a-cyano-4-hydroxy-cinnamic acid (ACHA) matrix was applied to a Bruker 384-spot MALDI target by spreading 5 μl of a saturated solution in MeCN/water (2/1, v/v) over an entire row of spots on the target; drying occurred in 2-5 sec. The IAP eluate (10 μl) was loaded onto an 0.2 μl C-18 ZipTip (Millipore), which then was washed with 5% formic acid. Peptide was eluted with 1 μl of 10 mg/ml ACHA in 60% methanol, 5% formic acid onto the MALDI target containing the thin layer of matrix. Samples were analyzed on a Bruker BiFlex III MALDI-TOF instrument in positive ion mode.

### Analysis by LC-MS/MS Mass Spectrometry.

40 μl of IAP eluate were purified by 0.2 μl Stage tips. Peptides were eluted from the microcolumns with 1 μl of 40% MeCN, 0.1% TFA (fractions I and II) or 1 μl of 60% MeCN, 0.1% TFA (fraction III) into 7.6 μl of 0.4% acetic acid/0.005% heptafluorobutyric acid. This sample was loaded onto a 10 cm×75 μm PicoFrit capillary column (New Objective) packed with Magic C18 AQ reversed-phase resin (Michrom Bioresources) using a Famos autosampler with an inert sample injection valve (Dionex). The column was then developed with a 45-min linear gradient of acetonitrile delivered at 200 nl/min (Ultimate, Dionex), and tandem mass spectra were collected in a data-dependent manner with an LCQ Deca XP Plus ion trap mass spectrometer essentially as described by Gygi et al., supra.

### Database Analysis & Assignments.

MS/MS spectra were evaluated using TurboSequest in the Sequest Browser package (v. 27, rev. 12) supplied as part of BioWorks 3.0 (ThermoFinnigan). Individual MS/MS spectra were extracted from the raw data file using the Sequest Browser program CreateDta, with the following settings: bottom MW, 700; top MW, 4,500; minimum number of ions, 20; minimum TIC, 4×10⁵; and precursor charge state, unspecified. Spectra were extracted from the beginning of the raw data file before sample injection to the end of the eluting gradient. The lonQuest and VuDta programs were not used to further select MS/MS spectra for Sequest analysis. MS/MS spectra were evaluated with the following TurboSequest parameters: peptide mass tolerance, 2.5; fragment ion tolerance, 0.0; maximum number of differential amino acids per modification, 4; mass type parent, average; mass type fragment, average; maximum number of internal cleavage sites, 10; neutral losses of water and ammonia from b and y ions were considered in the correlation analysis. Proteolytic enzyme was specified except for spectra collected from elastase digests.

Searches were performed against the NCBI human protein database (NCBI RefSeq protein release #11; 8 May 2005; 1,826,611 proteins, including 47,859 human proteins. Peptides that did not match RefSeq were compared to NCBI GenPept release #148; 15 Jun. 2005 release date; 2,479,172 proteins, including 196,054 human proteins.). Cysteine carboxamidomethylation was specified as a static modification, and phosphorylation was allowed as a variable modification on serine, threonine, and tyrosine residues or on tyrosine residues alone. It was determined that restricting phosphorylation to tyrosine residues had little effect on the number of phosphorylation sites assigned. Furthermore, it should be noted that certain peptides were originally isolated in mouse and later normalized to human sequences as shown by Table 1/FIG. 2.

In proteomics, it is desirable to validate protein identifications based solely on the observation of a single peptide in one experimental result, in order to indicate that the protein is, in fact, present in a sample. This has led to the development of statistical methods for validating peptide assignments, which are not yet universally accepted, and guidelines for the publication of protein and peptide identification results (see Carr et al. Mol Cell Proteomics 3: 531-533 (2004), which were followed in this Example. However, because the immunoaffinity strategy separates phosphorylated peptides from unphosphorylated peptides, observing just one phosphopeptide from a protein is a common result, since many phosphorylated proteins have only one tyrosine-phosphorylated site. For this reason, it is appropriate to use additional criteria to validate phosphopeptide assignments. Assignments are likely to be correct if any of these additional criteria are met: (i) the same sequence is assigned to co-eluting ions with different charge states, since the MS/MS spectrum changes markedly with charge state; (ii) the site is found in more than one peptide sequence context due to sequence overlaps from incomplete proteolysis or use of proteases other than trypsin; (iii) the site is found in more than one peptide sequence context due to homologous but not identical protein isoforms; (iv) the site is found in more than one peptide sequence context due to homologous but not identical proteins among species; and (v) sites validated by MS/MS analysis of synthetic phosphopeptides corresponding to assigned sequences, since the ion trap mass spectrometer produces highly reproducible MS/MS spectra. The last criterion is routinely employed to confirm novel site assignments of particular interest.

All spectra and all sequence assignments made by Sequest were imported into a relational database. The following Sequest scoring thresholds were used to select phosphopeptide assignments that are likely to be correct: RSp<6, XCorr≧2.2, and DeltaCN>0.099. Further, the assigned sequences could be accepted or rejected with respect to accuracy by using the following conservative, two-step process.

In the first step, a subset of high-scoring sequence assignments should be selected by filtering for XCorr values of at least 1.5 for a charge state of +1, 2.2 for +2, and 3.3 for +3, allowing a maximum RSp value of 10. Assignments in this subset should be rejected if any of the following criteria were satisfied: (i) the spectrum contains at least one major peak (at least 10% as intense as the most intense ion in the spectrum) that can not be mapped to the assigned sequence as an a, b, or y ion, as an ion arising from neutral-loss of water or ammonia from a b or y ion, or as a multiply protonated ion; (ii) the spectrum does not contain a series of b or y ions equivalent to at least six uninterrupted residues; or (iii) the sequence is not observed at least five times in all the studies conducted (except for overlapping sequences due to incomplete proteolysis or use of proteases other than trypsin).

In the second step, assignments with below-threshold scores should be accepted if the low-scoring spectrum shows a high degree of similarity to a high-scoring spectrum collected in another study, which simulates a true reference library-searching strategy.

### EXAMPLE 2

### Production of Phospho-specific Polyclonal Antibodies for the Detection of ALCL-Related Protein Phosphorylation

Polyclonal antibodies that specifically bind an ALCL-related signal transduction protein only when phosphorylated at the respective phosphorylation site disclosed herein (see Table 1) are produced according to standard methods by first constructing a synthetic peptide antigen comprising the phosphorylation site sequence and then immunizing an animal to raise antibodies against the antigen, as further described below. Production of exemplary polyclonal antibodies is provided below.

### A. MAPK6 (Tyrosine 628).

A 17 amino acid phospho-peptide antigen, KDEQVEKENTYTSy*LDK (SEQ ID NO: 100) (where y*=phosphotyrosine), that corresponds to the tyrosine 628 phosphorylation site in human anaplastic lymphoma kinase (ALK) (see Row 100 of Table 1), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific MAPK6(tyr628) polyclonal antibodies as described in Immunization/Screening below.

### B. GAB3 (Tyrosine 395).

A 15 amino acid phospho-peptide antigen, SPSAEDSy*VPMSPKG (SEQ ID NO: 9) (where y*=phosphotyrosine), that corresponds to the tyrosine 395 phosphorylation site in human GAB3 (see Row 9 of Table 1), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific GAB3 (tyr395) polyclonal antibodies as described in Immunization/Screening below.

### C. PPP2CB (Tyrosine 284).

A 26 amino acid phospho-peptide antigen, CGNQAAIMELDDTLKy*SFLQFDPAPR (SEQ ID NO: 127) (where y*=phosphotyrosine) that corresponds to the tyrosine 284 phosphorylation site in human PPP2CB (see Row 127 of Table 1), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific PPP2CB(tyr284) antibodies as described in Immunization/Screening below.

### Immunization/Screening.

A synthetic phospho-peptide antigen as described in A-C above is coupled to KLH, and rabbits are injected intradermally (ID) on the back with antigen in complete Freunds adjuvant (500 μg antigen per rabbit). The rabbits are boosted with same antigen in incomplete Freund adjuvant (250 μg antigen per rabbit) every three weeks. After the fifth boost, bleeds are collected. The sera are purified by Protein A-affinity chromatography by standard methods (see ANTIBODIES: A LABORATORY MANUAL, Cold Spring Harbor, supra.). The eluted immunoglobulins are further loaded onto a non-phosphorylated synthetic peptide antigen-resin Knotes column to pull out antibodies that bind the non-phosphorylated form of the phosphorylation site. The flow through fraction is collected and applied onto a phospho-synthetic peptide antigen-resin column to isolate antibodies that bind the phosphorylated form of the site. After washing the column extensively, the bound antibodies (i.e. antibodies that bind a phosphorylated peptide described in A-C above, but do not bind the non-phosphorylated form of the peptide, are eluted and kept in antibody storage buffer.

The isolated antibody is then tested for phospho-specificity using Western blot assay using an appropriate cell line the expresses (or overexpresses) target phospho-protein phosphorylated MAPK6, GABS or PPP2CB). Cells are cultured in DMEM supplemented with 10% FCS and 5 U/ml IL-3. Before stimulation, the cells are starved in serum-free DMEM medium for 4 hours. The cells are then stimulated ligand (e.g. 50 ng/ml) for 5 minutes. Cell are collected, washed with PBS and directly lysed in cell lysis buffer. The protein concentration of cell lysates are then measured. The loading buffer is added into cell lysate and the mixture is boiled at 100° C. for 5 minutes. 20 μl (10 μg protein) of sample is then added onto 7.5% SDS-PAGE gel.

A standard Western blot may be performed according to the Immunoblotting Protocol set out in the CELL SIGNALING TECHNOLOGY, INC. 2003-04 Catalogue, p. 390. The isolated phospho-specific antibody is used at dilution 1:1000. Phosphorylation-site specificity of the antibody will be shown by binding of only the phosphorylated form of the target protein. Isolated phospho-specific polyclonal antibody does not recognize the target protein when not phosphorylated at the appropriate phosphorylation site in the non-stimulated cells (e.g., MAPK6 is not bound when not phosphorylated at tyrosine 628).

In order to confirm the specificity of the isolated antibody, different cell lysates containing various phosphorylated signal transduction proteins other than the target protein are prepared. The Western blot assay is preformed again using these cell lysates. The phospho-specific polyclonal antibody isolated as described above is used (1:1000 dilution) to test reactivity with the different phosphorylated non-target proteins on Western blot membrane. The phospho-specific antibody does not significantly cross-react with other phosphorylated signal transduction proteins, although occasionally slight binding with a highly-homologous phosphorylation-site on another protein may be observed. In such case the antibody may be further purified using affinity chromatography, or the specific immunoreactivity cloned by rabbit hybridoma technology.

### EXAMPLE 3

### Production of Phospho-specific Monoclonal Antibodies for the Detection of ALCL-related Protein Phosphorylation

Monoclonal antibodies that specifically bind an ALCL-related signal transduction protein only when phosphorylated at the respective phosphorylation site disclosed herein (see Table 1) are produced according to standard methods by first constructing a synthetic peptide antigen comprising the phosphorylation site sequence and then immunizing an animal to raise antibodies against the antigen, and harvesting spleen cells from such animals to produce fusion hybridomas, as further described below. Production of exemplary monoclonal antibodies is provided below.

### A. CAMK1 (Tyrosine 184).

A 15 amino acid phospho-peptide antigen, TACGTPGy*VAPEVLA (SEQ ID NO: 96) (where y*=phosphotyrosine) that corresponds to the tyrosine 184 phosphorylation site in human CAMK4 (see Row 96 of Table 1), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal CAMK4(tyr184) antibodies as described in Immunization/Fusion/Screening below.

### B. IRS4 (Tyrosine 111).

A 17 amino acid phospho-peptide antigen, LETADAPARLEy*YENAR (SEQ ID NO: 12) (where y*=phosphotyrosine) that corresponds to the tyrosine 111 phosphorylation site in human IRS4 (see Row 12 of Table 1), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal IRS4(tyr111) antibodies as described in Immunization/Fusion/Screening below.

### C. PTPN11 (Tyrosine 66).

A 15 amino acid phospho-peptide antigen, IQNTGDYYDLy*GGEK (SEQ ID NO: 128) (where y*=phosphotyrosine) that corresponds to the tyrosine 66 phosphorylation site in human PTPN11 (see Row 128 of Table 1), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal PTPN11(tyr66) antibodies as described in Immunization/Fusion/Screening below.

### Immunization/Fusion/Screening.

A synthetic phospho-peptide antigen as described in A-C above is coupled to KLH, and BALB/C mice are injected intradermally (ID) on the back with antigen in complete Freunds adjuvant (e.g. 50 μg antigen per mouse). The mice are boosted with same antigen in incomplete Freund adjuvant (e.g. 25 μg antigen per mouse) every three weeks. After the fifth boost, the animals are sacrificed and spleens are harvested.

Harvested spleen cells are fused to SP2/0 mouse myeloma fusion partner cells according to the standard protocol of Kohler and Milstein (1975). Colonies originating from the fusion are screened by ELISA for reactivity to the phospho-peptide and non-phospho-peptide forms of the antigen and by Western blot analysis (as described in Example 1 above). Colonies found to be positive by ELISA to the phospho-peptide while negative to the non-phospho-peptide are further characterized by Western blot analysis. Colonies found to be positive by Western blot analysis are subcloned by limited dilution. Mouse ascites are produced from a single clone obtained from subcloning, and tested for phospho-specificity (against the CAMK1, IRS4, or PTPN11 phospho-peptide antigen, as the case may be) on ELISA. Clones identified as positive on Western blot analysis using cell culture supernatant as having phospho-specificity, as indicated by a strong band in the induced lane and a weak band in the uninduced lane of the blot, are isolated and subcloned as clones producing monoclonal antibodies with the desired specificity.

Ascites fluid from isolated clones may be further tested by Western blot analysis. The ascites fluid should produce similar results on Western blot analysis as observed previously with the cell culture supernatant, indicating phospho-specificity against the phosphorylated target (e.g. CAMK1 phosphorylated at tyrosine 184).

### EXAMPLE 4

### Production and Use of AQUA Peptides for the Quantification of ALCL-Related Signaling Protein Phosphorylation

Heavy-isotope labeled peptides (AQUA peptides (internal standards)) for the detection and quantification of an ALCL-related signal transduction protein only when phosphorylated at the respective phosphorylation site disclosed herein (see Table 1) are produced according to the standard AQUA methodology (see Gygi et al., Gerber et al., supra.) methods by first constructing a synthetic peptide standard corresponding to the phosphorylation site sequence and incorporating a heavy-isotope label. Subsequently, the MSⁿ and LC-SRM signature of the peptide standard is validated, and the AQUA peptide is used to quantify native peptide in a biological sample, such as a digested cell extract. Production and use of exemplary AQUA peptides is provided below.

### A. BMX (Tyrosine 194).

An AQUA peptide having a sequence corresponding to the tyrosine 334 phosphorylation site in human BMX, ILPQYDSySKKSCGS (y*=phosphotyrosine) (see Row 107 in Table 1 (SEQ ID NO: 107)) but incorporating ¹⁴C/¹⁵N-labeled leucine (indicated by bold L) is constructed according to standard synthesis techniques using, e.g., a Rainin/Protein Technologies, Inc., Symphony peptide synthesizer (see Merrifield, supra.) as further described below in Synthesis & MS/MS Signature. The BMX(tyr194) AQUA peptide is then spiked into a biological sample to quantify the amount of phosphorylated BMX (tyr194) in the sample, as further described below in Analysis & Quantification.

### B. CBLB (Tyrosine 276).

An AQUA peptide having a sequence corresponding to the tyrosine 858 phosphorylation site in human CBLB, ARLQKySTKPGSYIFR (y*=phosphotyrosine) (see Row 23 in Table 1 (SEQ ID NO: 23)) but incorporating ¹⁴C/¹⁵N-labeled proline (indicated by bold P) is constructed according to standard synthesis techniques using, e.g., a Rainin/Protein Technologies, Inc., Symphony peptide synthesizer (see Merrifield, supra.) as further described below in Synthesis & MS/MS Signature. The CBLB(tyr276) AQUA peptide is then spiked into a biological sample to quantify the amount of phosphorylated CBLB (tyr276) in the sample, as further described below in Analysis & Quantification.

### C. GATA6 (Tyrosine 417).

An AQUA peptide having a sequence corresponding to the tyrosine 654 phosphorylation site in human Enolase alpha, DGTGHYLCNACGLySK (y*=phosphotyrosine) (see Row 168 in Table 1 (SEQ ID NO: 168)) but incorporating ¹⁴C/¹⁵N-labeled leucine (indicated by bold L) is constructed according to standard synthesis techniques using, e.g., a Rainin/Protein Technologies, Inc., Symphony peptide synthesizer (see Merrifield, supra.) as further described below in Synthesis & MS/MS Signature. The GATA6 (tyr417) AQUA peptide is then spiked into a biological sample to quantify the amount of phosphorylated GATA6(tyr417) in the sample, as further described below in Analysis & Quantification.

### D. USP11 (Tyrosine 870).

An AQUA peptide having a sequence corresponding to the tyrosine 56 phosphorylation site in human USP11, DLDFSEFVIQPQNESNPELy*K (r=phosphotyrosine) (see Row 214 in Table 1 (SEQ ID NO: 214)) but incorporating ¹⁴C/¹⁵N-labeled leucine (indicated by bold L) is constructed according to standard synthesis techniques using, e.g., a Rainin/Protein Technologies, Inc., Symphony peptide synthesizer (see Merrifield, supra.) as further described below in Synthesis & MS/MS Signature. The USP11(tyr870) AQUA peptide is then spiked into a biological sample to quantify the amount of phosphorylated USP11(tyr870) in the sample, as further described below in Analysis & Quantification.

### Synthesis & MS/MS Spectra.

Fluorenylmethoxycarbonyl (Fmoc)-derivatized amino acid monomers may be obtained from AnaSpec (San Jose, Calif.). Fmoc-derivatized stable-isotope monomers containing one ¹⁵N and five to nine ¹³C atoms may be obtained from Cambridge Isotope Laboratories (Andover, Mass.). Preloaded Wang resins may be obtained from Applied Biosystems. Synthesis scales may vary from 5 to 25 μmol. Amino acids are activated in situ with 1-H-benzotriazolium, 1-bis(dimethylamino) methylenej-hexafluorophosphate(1-),3-oxide:1-hydroxybenzotriazole hydrate and coupled at a 5-fold molar excess over peptide. Each coupling cycle is followed by capping with acetic anhydride to avoid accumulation of one-residue deletion peptide byproducts. After synthesis peptide-resins are treated with a standard scavenger-containing trifluoroacetic acid (TFA)-water cleavage solution, and the peptides are precipitated by addition to cold ether. Peptides (i.e. a desired AQUA peptide described in A-D above) are purified by reversed-phase C18 HPLC using standard TFA/acetonitrile gradients and characterized by matrix-assisted laser desorption ionization-time of flight (Biflex III, Bruker Daltonics, Billerica, Mass.) and ion-trap (ThermoFinnigan, LCQ DecaXP) MS.

MS/MS spectra for each AQUA peptide should exhibit a strong y-type ion peak as the most intense fragment ion that is suitable for use in an SRM monitoring/analysis. Reverse-phase microcapillary columns (0.1 Å˜150-220 mm) are prepared according to standard methods. An Agilent 1100 liquid chromatograph may be used to develop and deliver a solvent gradient [0.4% acetic acid/0.005% heptafluorobutyric acid (HFBA)/7% methanol and 0.4% acetic acid/0.005% HFBA/65% methanol/35% acetonitrile] to the microcapillary column by means of a flow splitter. Samples are then directly loaded onto the microcapillary column by using a FAMOS inert capillary autosampler (LC Packings, San Francisco) after the flow split. Peptides are reconstituted in 6% acetic acid/0.01% TFA before injection.

### Analysis & Quantification.

Target protein (e.g. a phosphorylated protein of A-D above) in a biological sample is quantified using a validated AQUA peptide (as described above). The IAP method is then applied to the complex mixture of peptides derived from proteolytic cleavage of crude cell extracts to which the AQUA peptides have been spiked in.

LC-SRM of the entire sample is then carried out. MS/MS may be performed by using a ThermoFinnigan (San Jose, Calif.) mass spectrometer (LCQ DecaXP ion trap or TSQ Quantum triple quadrupole). On the DecaXP, parent ions are isolated at 1.6 m/z width, the ion injection time being limited to 150 ms per microscan, with two microscans per peptide averaged, and with an AGC setting of 1×10⁸; on the Quantum, Q1 is kept at 0.4 and Q3 at 0.8 m/z with a scan time of 200 ms per peptide. On both instruments, analyte and internal standard are analyzed in alternation within a previously known reverse-phase retention window; well-resolved pairs of internal standard and analyte are analyzed in separate retention segments to improve duty cycle. Data are processed by integrating the appropriate peaks in an extracted ion chromatogram (60.15 m/z from the fragment monitored) for the native and internal standard, followed by calculation of the ratio of peak areas multiplied by the absolute amount of internal standard (e.g., 500 fmol).

1. (canceled)

2. (canceled)

3. The method of claim 1, wherein said protein is a Kinase Protein selected from Column A, Rows 89-109, of Table 1, and wherein
(i) said antibody specifically binds said Kinase Protein only when phosphorylated at the tyrosine listed in corresponding Column D, Rows 89-109, of Table 1, comprised within the phosphorylation site sequence listed in corresponding Column E, Rows 89-109, of Table 1 (SEQ ID NOs: 89-107 and 109), and
(ii) said labeled peptide comprises the phosphorylation site sequence listed in corresponding Column E, Rows 89-109, of Table 1 (SEQ ID NOs: 89-107 and 109), comprising the phosphorylated tyrosine listed in corresponding Column D, Rows 89-109, of Table 1.

4. (canceled)

5. (canceled)

6. (canceled)

7. (canceled)

8. (canceled)

9. (canceled)

10. (canceled)

11. (canceled)

12. (canceled)

13. (canceled)

14. An isolated phosphorylation site-specific antibody that specifically binds a human Anaplastic Large Cell Lymphoma (ALCL)-related signaling protein selected from Column A of Table 1 only when phosphorylated at the tyrosine listed in corresponding Column D of Table 1, comprised within the phosphorylatable peptide sequence listed in corresponding Column E of Table 1 (SEQ ID NOs: 1-15, 17-39, 41-48, 50-64, 66-107, 109-148, 151-191, 193-215, 217-219), wherein said antibody does not bind said signaling protein when not phosphorylated at said tyrosine.

15. An isolated phosphorylation site-specific antibody that specifically binds a human ALCL-related signaling protein selected from Column A of Table 1 only when not phosphorylated at the tyrosine listed in corresponding Column D of Table 1, comprised within the phosphorylatable peptide sequence listed in corresponding Column E of Table 1 (SEQ ID NOs: 1-15, 17-39, 41-48, 50-64, 66-107, 109-148, 151-191, 193-215, 217-219), wherein said antibody does not bind said signaling protein when phosphorylated at said tyrosine.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. An isolated phosphorylation site-specific antibody according to claim 14, that specifically binds a human ALCL-related signaling protein selected from Column A, Rows 116, 2, 182, 153, 136 and 45 of Table 1 only when phosphorylated at the tyrosine listed in corresponding Column D of Table 1, comprised within the phosphorylatable peptide sequence listed in corresponding Column E of Table 1 (SEQ ID NOs: 115, 1, 181, 152, 135 and 44), wherein said antibody does not bind said signaling protein when not phosphorylated at said tyrosine.

48. An isolated phosphorylation site-specific antibody according to claim 15, that specifically binds a human ALCL-related signaling protein selected from Column A, Rows 116, 2, 182, 153, 136 and 45 of Table 1 only when not phosphorylated at the tyrosine listed in corresponding Column D of Table 1, comprised within the phosphorylatable peptide sequence listed in corresponding Column E of Table 1 (SEQ ID NOs: SEQ ID NOs: 115, 1, 181, 152, 135 and 44), wherein said antibody does not bind said signaling protein when phosphorylated at said tyrosine.

49. A method selected from the group consisting of:
(a) a method for detecting a human ALCL-related signaling protein selected from Column A of Table 1, wherein said human ALCL-related signaling protein is phosphorylated at the tyrosine listed in corresponding Column D of Table 1, comprised within the phosphorylatable peptide sequence listed in corresponding Column E of Table 1 (SEQ ID NOs: 1-15, 17-39, 41-48, 50-64, 66-107, 109-148, 151-191, 193-215, 217-219), comprising the step of adding an isolated phosphorylation-specific antibody according to claim 14, to a sample comprising said human ALCL-related signaling protein under conditions that permit the binding of said antibody to said human ALCL-related signaling protein, and detecting bound antibody;
(b) a method for quantifying the amount of a human ALCL-related signaling protein listed in Column A of Table 1 that is phosphorylated at the corresponding tyrosine listed in Column D of Table 1, comprised within the phosphorylatable peptide sequence listed in corresponding Column E of Table 1 (SEQ ID NOs: 1-15, 17-39, 41-48, 50-64, 66-107, 109-148, 151-191, 193-215, 217-219), in a sample using a heavy-isotope labeled peptide (AQUA™ peptide), said labeled peptide comprising a phosphorylated tyrosine at said corresponding tyrosine listed Column D of Table 1, comprised within the phosphorylatable peptide sequence listed in corresponding Column E of Table 1 as an internal standard; and
(c) a method comprising step (a) followed by step (b).

50. The method of claim 49, wherein said isolated phosphorylation-specific antibody is capable of specifically binding KLC2 only when phosphorylated at Y345, comprised within the phosphorylatable peptide sequence listed in Column E, Row 116, of Table 1 (SEQ ID NO: 115), wherein said antibody does not bind said protein when not phosphorylated at said tyrosine.

51. The method of claim 49, wherein said isolated phosphorylation-specific antibody is capable of specifically binding KLC2 only when not phosphorylated at Y345, comprised within the phosphorylatable peptide sequence listed in Column E, Row 116, of Table 1 (SEQ ID NO: 115), wherein said antibody does not bind said protein when phosphorylated at said tyrosine.

52. The method of claim 49, wherein said isolated phosphorylation-specific antibody is capable of specifically binding PCAF only when phosphorylated at Y729, comprised within the phosphorylatable peptide sequence listed in Column E, Row 2, of Table 1 (SEQ ID NO:1), wherein said antibody does not bind said protein when not phosphorylated at said tyrosine.

53. The method of claim 49, wherein said isolated phosphorylation-specific antibody is capable of specifically binding PCAF only when not phosphorylated at Y729, comprised within the phosphorylatable peptide sequence listed in Column E, Row 2, of Table 1 (SEQ ID NO:1), wherein said antibody does not bind said protein when phosphorylated at said tyrosine.

54. The method of claim 49, wherein said isolated, phosphorylation-specific antibody is capable of specifically binding SUI1 only when phosphorylated at Y79, comprised within the phosphorylatable peptide sequence listed in Column E, Row 182, of Table 1 (SEQ ID NO: 181), wherein said antibody does not bind said protein when not phosphorylated at said tyrosine.

55. The method of claim 49, wherein said isolated phosphorylation-specific antibody is capable of specifically binding SUI1 only when not phosphorylated at Y79, comprised within the phosphorylatable peptide sequence listed in Column E, Row 182, of Table 1 (SEQ ID NO: 181), wherein said antibody does not bind said protein when phosphorylated at said tyrosine.

56. The method of claim 49, wherein said isolated phosphorylation-specific antibody is capable of specifically binding HNRPL only when phosphorylated at Y441, comprised within the phosphorylatable peptide sequence listed in Column E, Row 153, of Table 1 (SEQ ID NO: 152), wherein said antibody does not bind said protein when not phosphorylated at said tyrosine.

57. The method of claim 49, wherein said isolated phosphorylation-specific antibody is capable of specifically binding HNRPL only when not phosphorylated at Y441, comprised within the phosphorylatable peptide sequence listed in Column E, Row 153, of Table 1 (SEQ ID NO: 152), wherein said antibody does not bind said protein when phosphorylated at said tyrosine.

58. The method of claim 49, wherein said isolated phosphorylation-specific antibody is capable of specifically binding PSMA7 only when phosphorylated at Y153, comprised within the phosphorylatable peptide sequence listed in Column E, Row 136, of Table 1 (SEQ ID NO: 135), wherein said antibody does not bind said protein when not phosphorylated at said tyrosine.

59. The method of claim 49, wherein said isolated phosphorylation-specific antibody is capable of specifically binding PSMA7 only when not phosphorylated at Y153, comprised within the phosphorylatable peptide sequence listed in Column E, Row 136, of Table 1 (SEQ ID NO: 135), wherein said antibody does not bind said protein when phosphorylated at said tyrosine.

60. The method of claim 49, wherein said isolated phosphorylation-specific antibody is capable of specifically binding VDAC1 only when phosphorylated at Y67, comprised within the phosphorylatable peptide sequence listed in Column E, Row 45, of Table 1 (SEQ ID NO: 44), wherein said antibody does not bind said protein when not phosphorylated at said tyrosine.

61. The method of claim 49, wherein said isolated phosphorylation-specific antibody is capable of specifically binding VDAC1 only when not phosphorylated at Y67, comprised within the phosphorylatable peptide sequence listed in Column E, Row 45, of Table 1 (SEQ ID NO: 44), wherein said antibody does not bind said protein when phosphorylated at said tyrosine.
